(19) United States
(12) Patent Application Publication
(10) Pub. No.: US 2006/0020004 A1
(43) Pub. Date: Jan. 26, 2006

(54) Isoxazolidine compounds for treatment of bacterial infections

(75) Inventors: Burton G. Christensen, Alamo, CA (US); Michael A. Foley, Chestnut Hill, MA (US); Asimina T. Georges Evangelinos, Andover, MA (US); Tao Liu, Ashland, MA (US); James R. Porter, Brighton, MA (US); Amy S. Ripka, Winthrop, MA (US); Linping Zhang, Lexington, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Infinity Pharmaceuticals, Inc., Cambridge, MA (US)

(21) Appl. No.: 11/155,314

(22) Filed: Jun. 17, 2005

Related U.S. Application Data
(60) Provisional application No. 60/580,418, filed on Jun. 17, 2004.

(51) Int. Cl. A61K 31/42; C07D 261/10; C07D 261/12; C07D 413/02

(52) U.S. Cl. 514/378; 548/240; 548/243

(57) ABSTRACT

The present invention relates to antibiotic compounds and intermediates useful in their preparation. Many of the antibiotic compounds contain a substituted isoxazolidine ring. The invention also relates to pharmaceutical compositions containing a compound of the invention. The invention further provides processes for the preparation of compounds of the invention, and methods for their use as therapeutic agents.

### RELATED APPLICATIONS

This application claims the benefit of priority to U.S. Provisional Patent Application Ser. No. 60/580,418, filed Jun. 17, 2004.

### FIELD OF THE INVENTION

The present invention is in the field of antimicrobial infections. The present invention specifically is in the field of isoxazolidine containing molecules and their use in treating bacterial infections in a mammal.

### BACKGROUND OF THE INVENTION

Antibacterial agents bestow enormous benefits to humanity and are credited with saving many millions of lives since their introduction in the 20th century. The more common antibacterial agents include penicillins, cephalosporins, tetracyclines, sulfonamides, aminoglycosides, glycopeptides, macrolides, monobactams, fluoroquinolones, quinolones, polymyxins, lincosamides, trimethoprim, and chloramphenicol. In general, bacterial pathogens may be classified as either Gram-positive or Gram-negative pathogens. Antibiotic compounds that are effective against both Gram-positive and Gram-negative pathogens are generally regarded as having a broad spectrum of activity.

Among the bacterial species that cause serious disease are the Gram-negative bacterium Escherichia coli and Gram-positive bacteria of the genus Staphylococcus. Staphylococcus aureus is the most serious pathogen of the Staphylococcus bacteria. It is estimated that Staphylococcus aureus causes 13% of the 2 million hospital infections each year, and results in 80,000 deaths in the United States. Staphylococcal infections often occur in patients weakened by poor health or immunodeficiency. Despite the large number of antibacterial agents that have been developed, many bacteria have become resistant to known antibiotics.

Several bacterial pathogens have demonstrated resistance to β-lactam antibiotics, such as penicillins and cephalosporins. Among the Gram-positive pathogens, Staphylococci, Enterococci, Streptococci, and mycobacteria are particularly important because resistant strains of these pathogens are difficult to treat and difficult to eradicate from the hospital environment once established. Representative examples of antibiotic-resistant Gram-positive bacteria are methicillin resistant staphylococcus (MRSA), methicillin resistant coagulase negative staphylococci (MRCNS), penicillin resistant Streptococcus pneumoniae (PRSP), and multiply resistant Enterococcus faecium, including vancomycin resistant Enterococcus spp. (VRE).

Enzymatic resistance to beta-lactam antibiotics is most often due to the enzyme beta-lactamase. Beta-lactamase enzymes catalyse the hydrolysis of amides, amidines, and other carbon and nitrogen bonds which inactivates beta-lactam antibiotics. The first beta-lactamase in common oral microorganisms was described on a plasmid in Haemophilus influenzae in the early 1970s. It carried the TEM-1 beta-lactamase first described in E. coli. The TEM-1 enzyme has been found in H. parainfluenzae and H. paraphrohaemolyticus and may be found in commensal Haemophilus species. The TEM-1 beta-lactamase is usually associated with large conjugative plasmids that are specific for the genus Haemophilus, which can also carry other genes for resistance to chloramphenicol, aminoglycosides and tetracycline. Since the discovery of this beta-lactamase, more than 190 unique beta-lactamases have been identified in Gram-positive and Gram-negative microorganisms.

The major, clinically-effective antibiotic for treatment of resistant Gram-positive pathogens is vancomycin. Vancomycin is a glycopeptide associated with nephrotoxicity and ototoxicity. Vancomycin works by binding to the terminal D-Ala-D-Ala residues of the cell wall peptidioglycan precursor. Unfortunately, there has been an emergence of vancomycin-resistant strains of Enterococci (Woodford N. 1998 Glycopeptide-resistant Enterococci: a decade of experience. Journal of Medical Microbiology. 47(10):849-62). Vancomycin-resistant Enterococci are particularly hazardous because they frequently cause hospital-based infections and are inherently resistant to most antibiotics. High-level resistance to vancomycin, known as VanA, is conferred by a gene located on a transposable element which alters the terminal residues to D-Ala-D-lac. Altering the terminal residues reduces the affinity for vancomycin. Importantly, resistance to vancomycin is increasing at a steady rate rendering it less and less effective in the treatment of Gram-positive pathogens.

In view of the rapid emergence of bacteria that are resistant to currently known antibiotic agents, the need exists for novel antibacterial agents that are effective against the growing number of resistant bacteria. Of particular importance is the need for antibiotic agents that can be used to treat patients infected with vancomycin-resistant Enterococci and methicillin-resistant Staphylocccus aureus.

### SUMMARY OF THE INVENTION

One aspect of the present invention relates to isoxazolidine compounds. In certain instances, the nitrogen atom of the isoxazolidine ring is bonded to a substituted aralkyl group. In certain instances, the substituted aralkyl group is a substituted benzyl group. In certain instances, the isoxazolidine ring is substituted with a hydroxy methyl or hydroxy ethyl group. In certain instances, isoxazolidine ring is substituted with a hydroxy methyl and a hydroxy ethyl group. In certain instances, the isoxazolidine ring is substituted with an amide group. The present invention further provides pharmaceutically active salts of the above-mentioned isoxazolidine compounds. Another aspect of the present invention relates to pharmaceutical compositions comprising an isoxazolidine compound of the invention. Another aspect of the present invention relates to a method of using the above compounds, or pharmaceutically active salts thereof, alone or in combination with other agents to treat bacterial infection. Specifically, the invention provides a therapeutic method comprising treating a bacterial infection of enterococci, pneumococci and methicillin resistant strains of S. aureus and coagulase negative staphylococci. In certain instances, the compound of the present invention is administered along with a pharmaceutically acceptable carrier.

### DETAILED DESCRIPTION OF THE INVENTION

The present invention generally relates to isoxazolidine compounds useful for treating bacterial infections. The isoxazolidine compounds of the invention show good activity against enterococci, pneumococci, and methicillin resistant strains of S. aureus and coagulase negative staphylococci. The isoxazolidine compounds of the invention can be used to treat a patient suffering from bacterial infection. In certain instances, the isoxazolidine compounds of the invention are used to treat a patient suffering from an infection of enterococci, pneumococci, or methicillin resistant strains of S. aureus or coagulase negative staphylococci. The isoxazolidine compounds of the invention can be administered to a patient in the form of a pharmaceutical composition. The pharmaceutical composition comprises the isoxazolidine compound of the invention and one or more pharmaceutically acceptable excipients.

In addition, the present invention provides methods for producing an antibacterial effect in a warm blooded animal, such as man, in need of such treatment, which comprises administering to said animal an effective amount of a compound of the present invention, or a pharmaceutically-acceptable salt. The compounds of the invention can be used in the manufacture of a medicament for treating bacterial infections. In order to use a compound of the present invention, a pharmaceutically-acceptable salt thereof, (hereinafter in this section relating to pharmaceutical composition “a compound of this invention”) for the therapeutic (including prophylactic) treatment of mammals including humans, in particular in treating infection, it is normally formulated in accordance with standard pharmaceutical practice as a pharmaceutical composition.

In certain instances, the isoxazolidine compounds of the invention may be co-administered (simultaneously, sequentially or separately) with one or more known drugs selected from other clinically useful antibacterial agents (for example, β-lactams or aminoglycosides) and/or other anti-infective agents (for example, an antifungal triazole or amphotericin). These may include carbapenems, for example meropenem or imipenem, to broaden the therapeutic effectiveness. Compounds of this invention may also contain or be co-administered with bactericidal/permeability-increasing protein (BPI) products or efflux pump inhibitors to improve activity against gram negative bacteria and bacteria resistant to antimicrobial agents. Furthermore, a pharmaceutical composition to be dosed intravenously may contain advantageously (for example to enhance stability) a suitable bactericide, antioxidant or reducing agent, or a suitable sequestering agent.

Synthesis of Isoxazolidine Compounds

The isoxazolidine compounds of the invention can be prepared using a 3+2 cycloaddition reaction between a nitrone and an alkene. The nitrone substrate and alkene may contain functional groups for further chemical derivatization following synthesis of the isoxazolidine core. In certain instances, a Lewis acid is added to the reaction. In a preferred embodiment, the Lewis acid is Ti(Oi-Pr)₄. In certain instances, the reaction mixture is subjected to microwave radiation. In general, the subject reactions are carried out in a liquid reaction medium. The reactions may be conducted in an aprotic solvent, preferably one in which the reaction ingredients are substantially soluble. Suitable solvents include ethers, such as diethyl ether, 1,2-dimethoxyethane, diglyme, t-butyl methyl ether, tetrahydrofuran and the like; halogenated solvents, such as chloroform, dichloromethane, dichloroethane, chlorobenzene, carbon tetrachloride, and the like; aliphatic or aromatic hydrocarbon solvents, such as benzene, xylene, toluene, hexane, pentane and the like; esters and ketones, such as ethyl acetate, acetone, and 2-butanone; polar aprotic solvents, such as acetonitrile, dimethylsulfoxide, dimethylformamide, pyridine, and the like; or combinations of two or more solvents. The reactions can be conducted at a variety of temperatures. Generally, the reactions conducted at lower temperatures will take longer to reach completion. In certain instances, the cycloaddition reaction is conducted in the range of about 15° C. to about 60° C. In certain instances, the cycloaddition reaction is conducted in the range of about 15° C. to about 30° C. In certain instances, the cycloaddition reaction is conducted at about room temperature. In certain instances, the cycloaddition reaction is conducted in the range of about 80° C. to about 150° C. In certain instances, the cycloaddition reaction is conducted in the range of about 90° C. to about 120° C. In certain instances, the cycloaddition reaction is conducted in the range of about 95° C. to about 105° C. In certain instances, the cycloaddition reaction is conducted using a substrate attached to a solid support. Following synthesis of the isoxazolidine core, the isoxazolidine compound may be derivatized using a variety of functionalization reactions known in the art. Representative examples include palladium coupling reactions to alkenylhalides or aryl halides, oxidations, reductions, reactions with nucleophiles, reactions with electrophiles, pericyclic reactions, installation of protecting groups, removal of protecting groups, and the like.

Biological Activity Analysis

Antibacterial Activity

The pharmaceutically-acceptable compounds of the present invention are useful antibacterial agents having a good spectrum of activity in vitro against standard gram-positive organisms, which are used to screen for activity against pathogenic bacteria. Notably, the pharmaceutically-acceptable compounds of the present invention show activity against enterococci, pneumococci and methicillin resistant strains of S. aureus and coagulase negative staphylococci. The antibacterial spectrum and potency of a particular compound may be determined in a standard test system.

The (antibacterial) properties of the compounds of the invention may also be demonstrated and assessed in vivo in conventional tests, for example by intravenous dosing of a compound to a warm-blooded mammal using standard techniques.

In Vitro Activity

Samples of the compounds prepared below in the Examples after solution in water and dilution with Nutrient Broth were found to exhibit Minimum Inhibitory Concentrations (MIC) values versus the indicated microorganisms as shown in Example 86. The MICs were determined using a broth micro-dilution assay in accordance with that recommended by the National Committee for Clinical Laboratory Standards (NCCLS). The final bacterial inoculate contained approximately 5×10⁵ cfu/mL and the plates were incubated at 35° C. for 18 h in ambient air (Streptococci in 5% CO₂). The MIC was defined as the lowest drug concentration that prevented visible growth.

In Vivo Activity

The in vivo therapeutic efficacy of the compounds prepared in the Examples below after intramuscular injection to mice experimentally infected with the representative MRSA strain A27223 was also measured.

Definitions

For convenience, certain terms employed in the specification, examples, and appended claims are collected here.

The term “heteroatom” as used herein means an atom of any element other than carbon or hydrogen. Preferred heteroatoms are boron, nitrogen, oxygen, phosphorus, sulfur and selenium.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 3-10 carbon atoms in their ring structure, and more preferably have 5, 6 or 7 carbons in the ring structure.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

The term “aralkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

The term “aryl” as used herein includes 5-, 6- and 7-membered single-ring aromatic groups that may include from zero to four heteroatoms, for example, benzene, anthracene, naphthalene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “amine” and “amino” are art-recognized and refer to both unsubstituted and substituted amines, e.g., a moiety that may be represented by the general formulas: 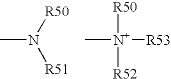
wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula: 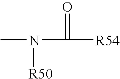
wherein R50 is as defined above, and R₅₄ represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula: 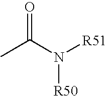
wherein R50 and R51 are as defined above. Certain embodiments of the amide in the present invention will not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carboxyl” is art recognized and includes such moieties as may be represented by the general formulas: 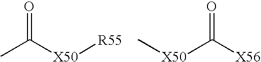
wherein X50 is a bond or represents an oxygen or a sulfur, and R55 and R56 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R₈, where m and R₈ are described above.

The term “sulfonate” is art recognized and includes a moiety that can be represented by the general formula: 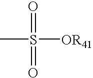
in which R₄₁ is an electron pair, hydrogen, alkyl, cycloalkyl, or aryl.

The term “carbamoyl” refers to —O(C═O)NRR′, where R and R′ are independently H, aliphatic groups, aryl groups or heteroaryl groups.

The term “alkylamino” refers to —NHR, where R is an alkyl group.

The term “dialkylamino” refers to —NRR′, where both R and R′ are alkyl groups.

The term “hydroxyalkyl” refers to —R—OH, where R is an aliphatic group.

The term “aminoalkyl” refers to —R—NH₂, where R is an aliphatic group.

The term “alkylaminoalkyl” refers to —R—NH—R′, where both R and R′ are aliphatic groups.

The term “dialkylaminoalkyl” refers to —R—N(R)—R″, where R, R, and R″ are aliphatic groups.

The term “arylaminoalkyl” refers to —R—NH—R′, where R is an aliphatic and R′ is an aryl group.

The term “oxo” refers to a carbonyl oxygen (═O).

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

The term “sulfate” is are recognized and includes a moiety that can be represented by the general formula: 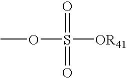
in which R₄₁ is as defined above.

The term “sulfonlamino” is art recognized and includes a moiety that can be represented

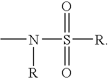

The term “sulfamoyl” is art-recognized and includes a moiety that can be represented by

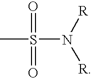

The term “sulfonyl”, as used herein, refers to a moiety that can be represented by the general formula: 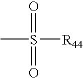
in which R₄₄ is selected from the group consisting of hydrogen, alkyl, alkenyl, alkynyl, cycloalkyl, heterocyclyl, aryl, or heteroaryl.

The term “sulfoxido” as used herein, refers to a moiety that can be represented by the general formula: 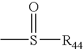
in which R₄₄ is selected from the group consisting of hydrogen, alkyl, alkenyl, alkynyl, cycloalkyl, heterocyclyl, aralkyl, or aryl.

A “selenoalkyl” refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R₇, m and R₇ being defined above.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g., alkyl, m, n, etc., when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991). Protected forms of the inventive compounds are included within the scope of this invention.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof (e.g., functioning as analgesics), wherein one or more simple variations of substituents are made which do not adversely affect the efficacy of the compound in binding to sigma receptors. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover.

The term “subject” as used herein, refers to an animal, typically a mammal or a human, that has been the object of treatment, observation, and/or experiment. When the term is used in conjunction with administration of a compound or drug, then the subject has been the object of treatment, observation, and/or administration of the compound or drug.

The term “pharmaceutically acceptable carrier” refers to a medium that is used to prepare a desired dosage form of a compound. A pharmaceutically acceptable carrier can include one or more solvents, diluents, or other liquid vehicles; dispersion or suspension aids; surface active agents; isotonic agents; thickening or emulsifying agents; preservatives; solid binders; lubricants; and the like. Remington's Pharmaceutical Sciences, Fifteenth Edition, E. W. Martin (Mack Publishing Co., Easton, Pa., 1975) and Handbook of Pharmaceutical Excipients, Third Edition, A. H. Kibbe ed. (American Pharmaceutical Assoc. 2000), disclose various carriers used in formulating pharmaceutical compositions and known techniques for the preparation thereof.

Compounds of the Invention

One aspect of the present invention relates to a compound represented by formula 1: 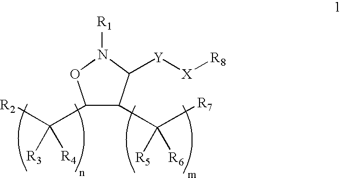
or pharmaceutically acceptable salts, solvates, or hydrates thereof,
wherein
Y is C(R)₂, —C(O)—, —C(S)—, or —S(O)₂—;
R represents independently for each occurrence H, alkyl, cycloalkyl, aralkyl, aryl or heteroaryl;
X is a bond, aryl, or NR₁₀;
m is 0, 1, 2, 3, 4, 5, or 6;
n represents independently for each occurrence 0, 1, 2, 3, 4, 5, or 6;
R₁ is alkyl, aralkyl, heteroaralkyl, —(C(R)₂)_{q}-cycloalkyl, —(C(R)₂)_{q}—Ar—CN, —(C(R)₂)_{q}—Ar—O(C(R)₂)_{q}-alkenyl, —(C(R)₂)_{q}-heterocycloalkyl-CO₂R, has the formula 1a: 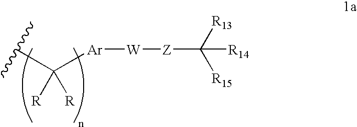
or has the formula 1b: 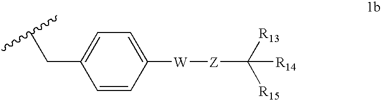
wherein
   W is a bond; or bivalent alkyl, alkenyl, or alkynyl chain;
   Z is a bond, —(C(R)₂)ₙ—, or —O(C(R)₂)ₙ—;
   R₁₃ and R₁₄ are independently H, alkyl, aryl, cycloalkyl, heterocycloalkyl, aralkyl, heteroaryl, or heteroaralkyl; or R₁₃ and R₁₄ taken together form a monocyclic or polycyclic ring; or R₁₃ and R₁₄ taken together with R₁₅ form a cycloalkenyl ring;
   R₁₅ is halide, hydroxyl, alkoxyl, aryloxy, acyloxy, amino, alkylamino, arylamino, acylamino, aralkylamino, nitro, acylthio, carboxamide, sulfonamide, carboxyl, nitrile, —COR, —CO₂R, —CH₂O-heterocyclyl, or —OR₁₉; or R₁₅ taken together with R₁₃ and R₁₄ form a cycloalkenyl ring; or has the formula 1c: 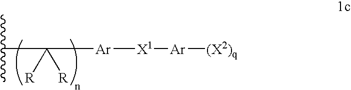
wherein
   X¹ is a bond, O, S, amino, alkylamino diradical, alkoxyl diradical, alkyl diradical, alkenyl diradical, alkynyl diradical, amido, sulfonamide, or carbonyl;
   X² represents independently for each occurrence H, hydroxyl, halide, thiol, nitrile, alkyl, fluoroalkyl, alkoxyl, aryl, —C(O)R₁₈, —CO₂R₁₈, —C(O)N(R₁₈)₂, —SO₂N(R)₂, —O-cycloalkyl, —O-heterocycloalkyl, —O-aryl, or —OR₁₉; and
   R₁₈ represents independently for each occurrence H, alkyl, aryl, cycloalkyl, heterocycloalkyl, aralkyl, heteroaryl, or heteroaralkyl; or two instances of R₁₈ taken together form a monocyclic or polycyclic ring;
Ar represents independently for each occurrence a monocyclic or bicyclic aryl with 6-14 ring atoms; or a monocyclic or bicyclic heteroaryl with 5-14 ring atoms, of which one, two or three ring atoms are independently S, O or N;
q represents independently for each occurrence 1, 2, 3, 4, or 5;
R₂ and R₇ represent independently H, hydroxyl, alkyl, alkoxyl, —N(R₁₁)₂, acylamino, —CO₂(C(R)₂)ₚC(R)(N(R)₂)CO₂R, —OP(O)(OR₁ 2)₂, —N(R)CO₂R, —OC(O)(C(R)₂)_{q}N(R₉)₂, —OC(O)(C(R)₂)_{q}CO₂R, —SO₂N(R)₂, or —OR₁₉; or R₂ and R₇ taken together form an optionally substituted alkyl or heteroalkyl linkage containing 1 to 6 carbon atoms; or R₇ is a bond to R₈; or R₇ and R₈ taken together form a ring comprising 4 to 7 atoms, of which one, two or three ring atoms may independently be S, O or N;
R₃ and R₆ each represent independently for each occurrence H, hydroxyl, —OC(O)R₉, alkyl, or —OR₁₉;
R₄ and R₅ each represent independently for each occurrence H or alkyl;
R₈ is a branched or unbranched alkyl or alkenyl, cycloalkyl, heterocycloalkyl, bicycloalkyl, a bond to R₇, or has the formula 1d: 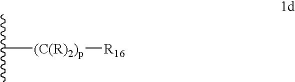
wherein
   p is 0, 1, 2, 3, 4, 5, or 6; and
   R₁₆ is hydroxyl, aryl, cycloalkyl, cycloalkenyl, heterocycloalkyl, alkoxyl, heteroaryl, —N(R₁₇)₂, —N(R)CO-alkyl, —N(R)CO₂-alkyl, —C(O)N(R₉)aryl, —SO₂N(R)₂, a polycyclic ring containing 8-14 carbon atoms, or —OR₁₉; wherein R₁₇ is independently for each occurrence H, alkyl, aryl, acyl, —(C(R)₂)_{q}OH, or —(C(R)₂)_{q}O(C(R)₂)_{q}OH; or two R₁₇ taken together form a ring;
   R₉ and R₁₀ each represent independently for each occurrence H, alkyl, aryl, cycloalkyl, aralkyl, heteroaryl, or heteroaralkyl;
   R₁₁ represents independently for each occurrence H, alkyl, cycloalkyl, aryl, or aralkyl;
   R₁₂ represents independently for each occurrence H, alkyl, aryl, aralkyl, or an alkali metal; 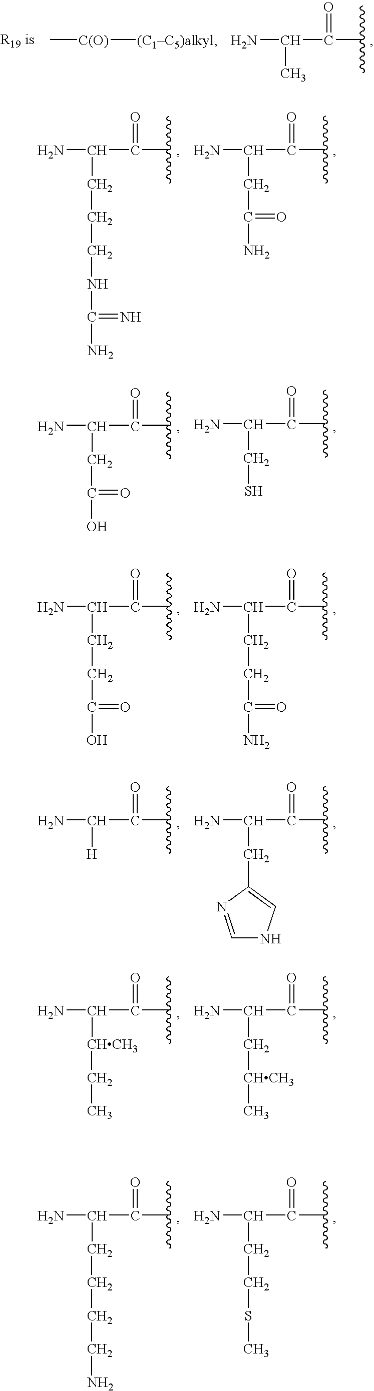 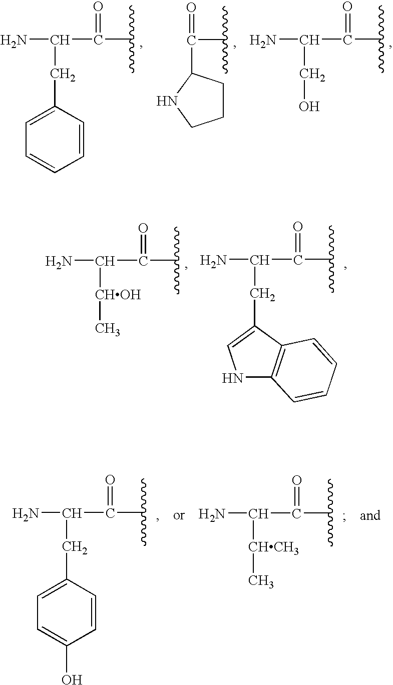
the stereochemical configuration at any stereocenter of a compound represented by 1 is R, S, or a mixture of these configurations.

Another aspect of the present invention relates to a compound represented by formula 2: 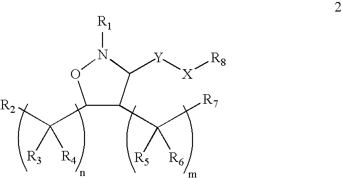
or pharmaceutically acceptable salts, solvates, or hydrates thereof,
wherein
Y is C(R)₂, —C(O)—, —C(S)—, or —S(O)₂—;
R represents independently for each occurrence H, alkyl, cycloalkyl, aralkyl, aryl or heteroaryl;
X is O, S, NR₁₀, or aryl;
m is 0, 1, 2, 3, 4, 5, or 6;
n represents independently for each occurrence 0, 1, 2, 3, 4, 5, or 6;
R₁ is alkyl, aralkyl, heteroaralkyl, —(C(R)₂)_{q}-cycloalkyl, has the formula 2a: 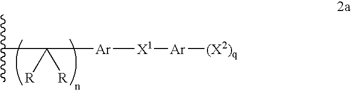
or has the formula 2b: 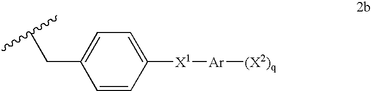
wherein
   Ar is a monocyclic or bicyclic aryl with 6-14 ring atoms; or a monocyclic or bicyclic heteroaryl with 5-14 ring atoms, of which one, two or three ring atoms are independently S, O or N;
   X¹ is a bond, O, S, amino, alkylamino diradical, alkoxyl diradical, alkyl diradical, alkenyl diradical, alkynyl diradical, amido, sulfonamide, or carbonyl;
   X² represents independently for each occurrence H, hydroxyl, halide, thiol, nitrile, alkyl, fluoroalkyl, —C(O)R₁₈, —CO₂R₁₈, —C(O)N(R₁₈)₂, —SO₂N(R)₂, —O-cycloalkyl, —O-heterocycloalkyl, —O-aryl, or —OR₁₉; and
   R₁₈ represents independently for each occurrence H, alkyl, aryl, cycloalkyl, heterocycloalkyl, aralkyl, heteroaryl, or heteroaralkyl; or two instances of R₁₈ taken together form a monocyclic or polycyclic ring;
q represents independently for each occurrence 1, 2, 3, 4, or 5;
R₂ and R₇ represent independently H, hydroxyl, alkyl, halide, alkoxyl, aryloxy, acyloxy, silyloxy, —N(R₁₁)₂, acylamino, —CO₂(C(R)₂)ₚC(R)(N(R)₂)CO₂R, —OP(O)(OR₁₂)₂, —N(R)CO₂R, —OC(O)(C(R)₂)_{q}N(R₉)₂, —OC(O)(C(R)₂)_{q}CO₂R, —SO₂N(R)₂, nitro, sulfhydryl, alkylthio, acylthio, carboxamide, carboxyl, silyl, thioalkyl, alkylsulfonyl, arylsulfonyl, alkylsulfonyloxy, arylsulfonyloxy, ketone, aldehyde, ester, nitrile, —CH₂O-heterocyclyl, or —OR₁₉; or R₂ and R₇ taken together form a —OC(O)O— linkage or an optionally substituted alkyl or heteroalkyl linkage containing 1 to 6 carbon atoms; or R₇ is a bond to R₈; or R₇ and R₈ taken together form a ring comprising 4 to 7 atoms, of which one, two or three ring atoms may independently be S, O or N;
R₃ and R₆ each represent independently for each occurrence H, halide, hydroxyl, amino, alkyl, aryl, cycloalkyl, aralkyl, heteroaryl, heteroaralkyl, alkoxyl, aryloxy, acyloxy, silyloxy, alkylamino, arylamino, acylamino, sulfonamide, aralklyamino, or —OR₁₉;
R₄ and R₅ each represent independently for each occurrence H, halide, alkyl, aryl, cycloalkyl, aralkyl, heteroaryl, heteroaralkyl, alkoxyl, aryloxy, acyloxy, silyloxy, alkylamino, arylamino, acylamino, sulfonamide, or aralklyamino;
R₈ is a branched or unbranched alkyl or alkenyl, cycloalkyl, heterocycloalkyl, bicycloalkyl, a bond to R₇, or has the formula 2c: 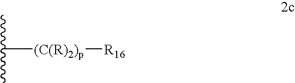
wherein
   p is 0, 1, 2, 3, 4, 5, or 6; and
   R₁₆ is aryl, cycloalkyl, cycloalkenyl, heterocyclyl, alkoxyl, heteroaryl, N(R₁₇)₂, —N(R)CO₂-alkyl, C(O)N(R₉)aryl, sulfonamide, or a polycyclic ring containing 8-14 carbon atoms; wherein R₁₇ is independently for each occurrence H, alkyl, aryl or acyl; or two R₁₇ taken together form a ring;
R₉ and R₁₀ each represent independently for each occurrence H, alkyl, aryl, cycloalkyl, aralkyl, heteroaryl, or heteroaralkyl;
R₁₁ represents independently for each occurrence H, alkyl, cycloalkyl, aryl, or aralkyl;
R₁₂ represents independently for each occurrence H, alkyl, aryl, aralkyl, or an alkali metal; 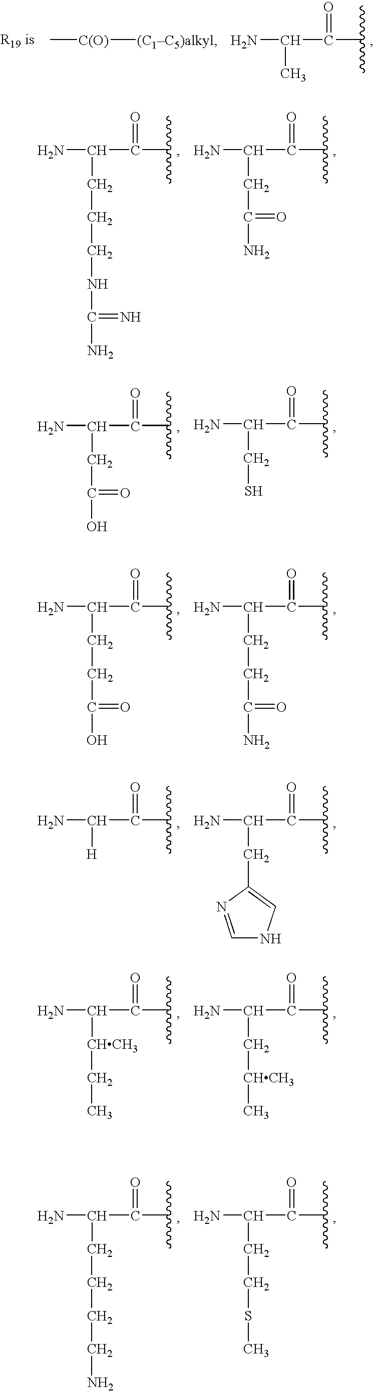 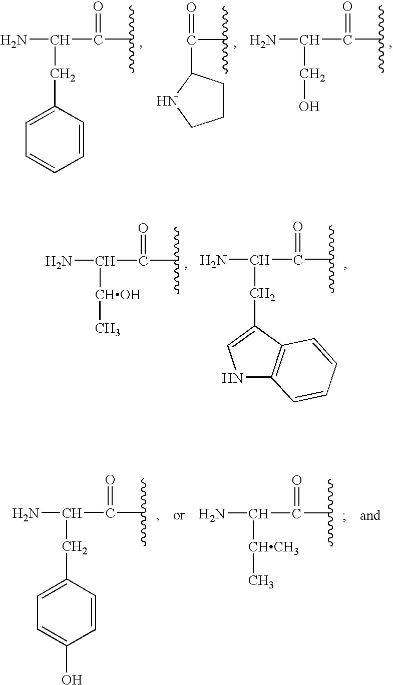
the stereochemical configuration at any stereocenter of a compound represented by 2 is R, S, or a mixture of these configurations.

In certain embodiments, the present invention relates to the aforementioned compound, wherein R₂ and R₇ are hydroxyl.

In certain embodiments, the present invention relates to the aforementioned compound, wherein R₂ and R₇ are hydroxyl; and R₄, R₅, and R₆ are H.

In certain embodiments, the present invention relates to the aforementioned compound, wherein R₂ and R₇ are hydroxyl; R₄, R₅, and R₆ are H; and m and n are 1.

In certain embodiments, the present invention relates to the aforementioned compound, wherein R₂ and R₇ are hydroxyl; R₄, R₅, R₆ are H; m and n are 1; and R₃ is methyl.

Another aspect of the present invention relates to a compound represented by formula 3: 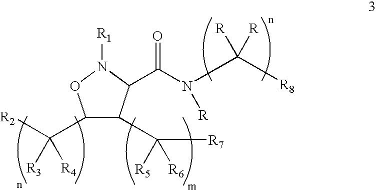
or pharmaceutically acceptable salts, solvates, or hydrates thereof,
wherein
m represents independently for each occurrence 0, 1, 2, 3, 4, 5, or 6;
n represents independently for each occurrence 0, 1, 2, 3, 4, 5, or 6;
R represents independently for each occurrence H, alkyl, cycloalkyl, aralkyl, aryl or heteroaryl;
R₁ is alkyl, aralkyl, heteroaralkyl, —(C(R)₂)_{q}-cycloalkyl, has the formula 3a: 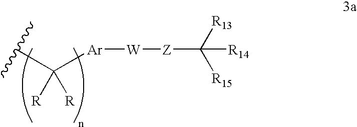
   or has the formula 3b: 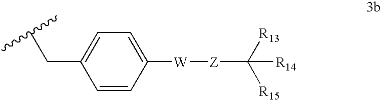
wherein
   W is a bond, or bivalent alkyl, alkenyl, or alkynyl chain;
   Z is a bond, —(C(R)₂)ₙ—, or —O(C(R)₂)ₙ—;
   R₁₃ and R₁₄ are independently H, halide, alkyl, cycloalkyl, alkenyl, alkynyl, aryl, aralkyl, heteroaryl, heteroaralkyl, aminoalkyl, thiol, thioalkyl, silyl, nitro, nitrile, alkoxyl, acyl, acylamino, sulfonamide, —COR, or —CO₂R; or R₁₃ and R₁₄ taken together form a monocyclic or polycyclic ring; or R₁₃ and R₁₄ taken together with R₁₅ form a cycloalkenyl ring; and
   R₁₅ is alkyl, cycloalkyl, aryl, heteroaryl, alkenyl, alkynyl, halide, hydroxyl, alkoxyl, aryloxy, acyloxy, silyloxy, amino, alkylamino, arylamino, acylamino, sulfonamide, aralkylamino, nitro, sulfhydryl, alkylthio, acylthio, carboxamide, carboxyl, phosphate, silyl, thioalkyl, alkylsulfonyl, arylsulfonyl, alkylsulfonyloxy, arylsulfonyloxy, nitrile, —COR, —CO₂R, —CH₂O-heterocyclyl, or —OR₁₉; or R₁₅ taken together with R₁₃ and R₁₄ form a cycloalkenyl ring; or has the formula 3b: 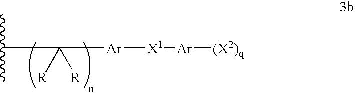
wherein
   X¹ is a bond, O, S, amino, alkylamino diradical, alkoxyl diradical, alkyl diradical, alkenyl diradical, alkynyl diradical, amido, sulfonamide, or carbonyl;
   X² represents independently for each occurrence H, hydroxyl, halide, thiol, nitrile, alkyl, fluoroalkyl, —C(O)R₁₈, —CO₂R₁₈, —C(O)N(R₁₈)₂, —SO₂N(R)₂, —O-cycloalkyl, —O-heterocycloalkyl, —O-aryl, or —OR₁₉; and
   R₁₈ represents independently for each occurrence H, alkyl, aryl, cycloalkyl, heterocycloalkyl, aralkyl, heteroaryl, or heteroaralkyl; or two instances of R₁₈ taken together form a monocyclic or polycyclic ring;
q is 1, 2, 3, 4, or 5;
Ar represents independently for each occurrence a monocyclic or bicyclic aryl with 6-14 ring atoms; or a monocyclic or bicyclic heteroaryl with 5-14 ring atoms, of which one, two or three ring atoms are independently S, O or N;
R₂ and R₇ each represent independently H, hydroxyl, alkyl, halide, alkoxyl, aryloxy, acyloxy, silyloxy, —N(R)₂, acylamino, —CO₂(C(R)₂)ₚC(R)(N(R)₂)CO₂R, —OP(O)(OR₁₂)₂, —N(R)CO₂R, —OC(O)(C(R)₂)_{q}N(R₉)₂, —OC(O)(C(R)₂)_{q}CO₂R, —SO₂N(R)₂, nitro, sulfhydryl, alkylthio, acylthio, carboxamide, carboxyl, silyl, thioalkyl, alkylsulfonyl, arylsulfonyl, alkylsulfonyloxy, arylsulfonyloxy, ketone, aldehyde, ester, nitrile, —CH₂O-heterocyclyl, or —OR, 9; or R₂ and R₇ taken together form a —OC(O)O— linkage or an optionally substituted alkyl or heteroalkyl linkage containing 1 to 6 carbon atoms; or R₇ is a bond to R₈; or R₇ and R₈ taken together form a ring comprising 4 to 7 atoms, of which one, two or three ring atoms may independently be S, O or N;
R₃ and R₆ each represent independently for each occurrence H, halide, hydroxyl, amino, alkyl, aryl, cycloalkyl, aralkyl, heteroaryl, heteroaralkyl, alkoxyl, aryloxy, acyloxy, silyloxy, alkylamino, arylamino, acylamino, sulfonamide, aralklyamino, or —OR₁₉;
R₄ and R₅ each represent independently for each occurrence H, halide, alkyl, aryl, cycloalkyl, aralkyl, heteroaryl, heteroaralkyl, alkoxyl, aryloxy, acyloxy, silyloxy, alkylamino, arylamino, acylamino, sulfonamide, or aralklyamino;
R₈ is aryl, heteroaryl, heterocycloalkyl, alkoxyl, or —N(R)CO-alkyl;
R₉ and R₁₀ each represent independently for each occurrence H, alkyl, aryl, cycloalkyl, aralkyl, heteroaryl, or heteroaralkyl;
R₁₁ represents independently for each occurrence H, alkyl, cycloalkyl, aryl, or aralkyl;
R₁₂ represents independently for each occurrence H, alkyl, aryl, aralkyl, or an alkali metal; 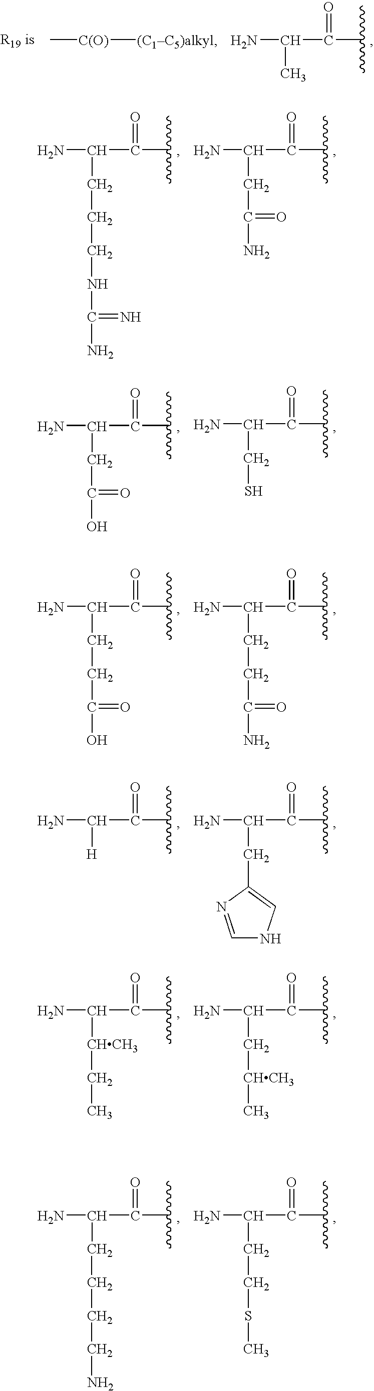 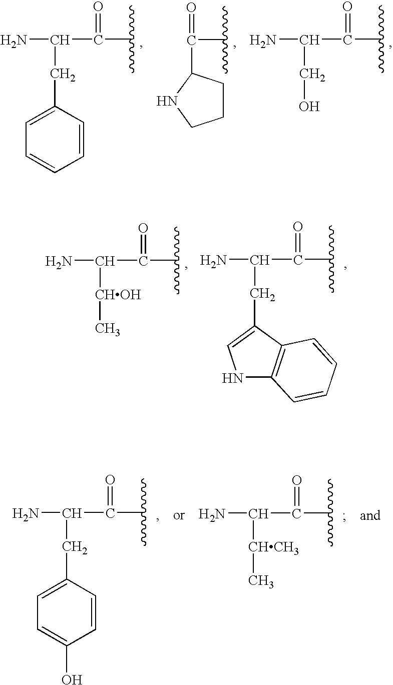
the stereochemical configuration at any stereocenter of a compound represented by 3 is R, S, or a mixture of these configurations.

In certain embodiments, the present invention relates to the aforementioned compound, wherein R₈ is selected from the group consisting of 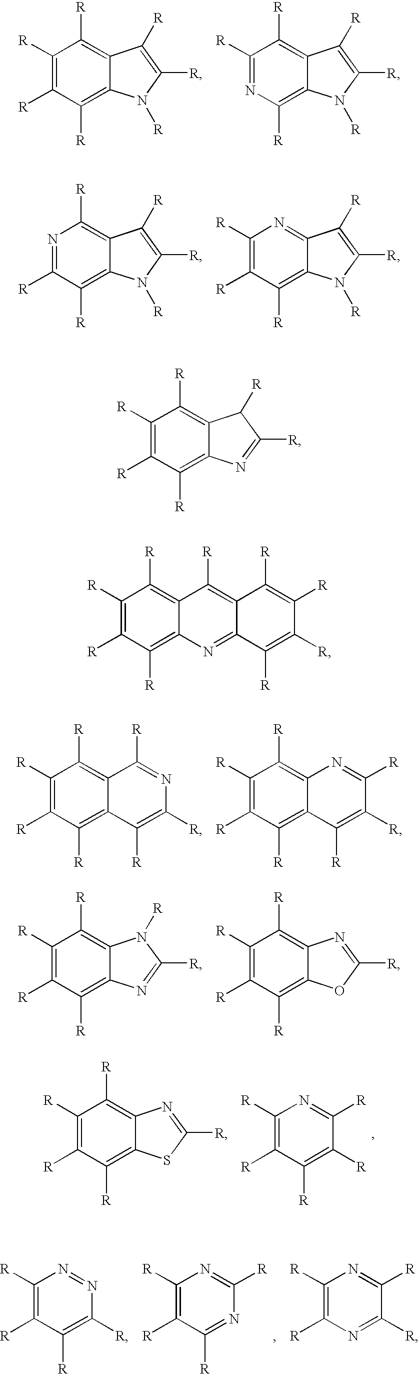 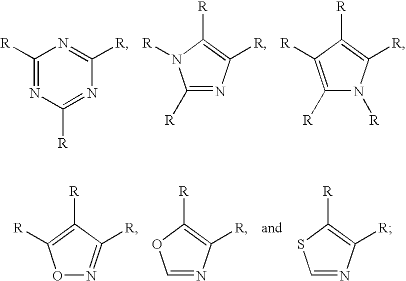
and R represents independently for each occurrence H, alkyl, aryl, or a bond to the CON(R)(CR₂)ₙ-fragment of the compound represented by formula 3.

In certain embodiments, the present invention relates to the aforementioned compound, wherein R₈ is 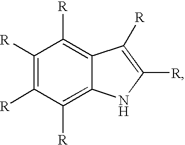
and R represents independently for each occurrence H, alkyl, aryl, or a bond to the CON(R)(CR₂)n-fragment of the compound represented by formula 3.

In certain embodiments, the present invention relates to the aforementioned compound, wherein R₈ is 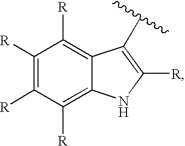
and R represents independently for each occurrence H or alkyl.

In certain embodiments, the present invention relates to the aforementioned compound, wherein R₈ is

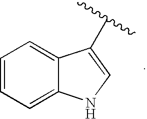

Another aspect of the present invention relates to a compound represented by formula 4: 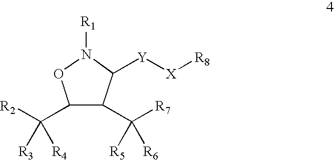
or pharmaceutically acceptable salts, solvates, or hydrates thereof,
wherein
Y is C(R)₂, —C(O)—, —C(S)—, or —S(O)₂—;
X is O, S, or NR₁₀;
R₁ is alkyl, aralkyl, heteroaralkyl, —(C(R)₂)_{q}-cycloalkyl, —(C(R)₂)_{q}—Ar—CN, —(C(R)₂)_{q}—Ar—O(C(R)₂)_{q}-alkenyl, —(C(R)₂)_{q}-heterocycloalkyl-CO₂R, has the formula 4a: 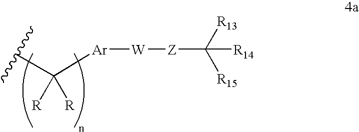
or has the formula 4b: 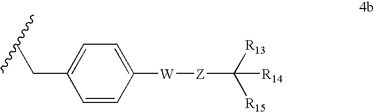
wherein
   W is a bond, or bivalent alkyl, alkenyl, or alkynyl chain;
   Z is a bond, —(C(R)₂)ₙ—, or —O(C(R)₂)ₙ—;
   R₁₃ and R₁₄ are independently H, alkyl, aryl, cycloalkyl, aralkyl, heteroaryl, or heteroaralkyl; or R₁₃ and R₁₄ taken together form a monocyclic or polycyclic ring; or R₁₃ and R₁₄ taken together with R₁₅ form a cycloalkenyl ring;
   R₁₅ is halide, hydroxyl, alkoxyl, aryloxy, acyloxy, silyloxy, amino, alkylamino, arylamino, acylamino, sulfonamide, aralkylamino, nitro, sulfhydryl, alkylthio, acylthio, carboxamide, carboxyl, phosphate, silyl, thioalkyl, alkylsulfonyl, arylsulfonyl, alkylsulfonyloxy, arylsulfonyloxy, nitrile, —COR, —CO₂R, —CH₂—O-heterocyclyl, or —OR₉; or R₁₅ taken together with R₁₃ and R₁₄ form a cycloalkenyl ring; or has the formula 4c: 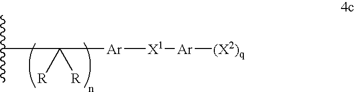
wherein
   X¹ is a bond, O, S, amino, alkylamino diradical, alkoxyl diradical, alkyl diradical, alkenyl diradical, alkynyl diradical, amido, sulfonamide, or carbonyl;
X² represents independently for each occurrence H, hydroxyl, halide, thiol, nitrile, alkyl, fluoroalkyl, —C(O)R₁₈, —CO₂R₁₈, —C(O)N(R₁₈)₂, —SO₂N(R)₂, —O-cycloalkyl, —O-heterocycloalkyl, or —O-aryl, —OR₁₉; and
   R₁₈ represents independently for each occurrence H, alkyl, aryl, cycloalkyl, heterocycloalkyl, aralkyl, heteroaryl, or heteroaralkyl; or two instances of R₁₈ taken together form a monocyclic or polycyclic ring;
q is 1, 2, 3, 4, or 5;
Ar represents independently for each occurrence a monocyclic or bicyclic aryl with 6-14 ring atoms; or a monocyclic or bicyclic heteroaryl with 5-14 ring atoms, of which one, two or three ring atoms are independently S, O or N;
R represents independently for each occurrence H, alkyl, cycloalkyl, aralkyl, aryl or heteroaryl;
R₂ and R₇ are independently hydroxyl, —N(R₁₁)₂, —OP(O)(OR₁₂)₂, alkoxyl, OC(O)(C(R)₂)_{q}N(R₉)₂, or —OR₁₉; or R₂ and R₇ taken together form a —OC(O)O— linkage or an optionally substituted alkyl or heteroalkyl linkage containing 1 to 6 carbon atoms;
R₃ is methyl, ethyl, or propyl;
R₄, R₅, and Rr are H;
R₈ is a branched or unbranched alkyl or alkenyl; cycloalkyl, heterocycloalkyl, bicycloalkyl, a bond to R₇, or has the formula 4d: 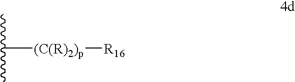
wherein
   p is 0, 1, 2, 3, 4, 5, or 6; and
   R₁₆ is aryl, cycloalkyl, cycloalkenyl, heterocyclyl, alkoxyl, heteroaryl, —N(R₁₇)₂, —N(R)CO₂-alkyl, sulfonamide, —C(O)N(R₉)aryl, or a polycyclic ring containing 8-14 carbon atoms; wherein R₁₇ is independently for each occurrence H, alkyl, aryl or acyl; or two R₁₇ taken together form a ring;
R₉ and R₁₀ each represent independently for each occurrence H, alkyl, aryl, cycloalkyl, aralkyl, heteroaryl, or heteroaralkyl;
R₁₁ represents independently for each occurrence H, alkyl, cycloalkyl, aryl, or aralkyl;
R₁₂ represents independently for each occurrence H, alkyl, aryl, aralkyl, or an alkali metal; 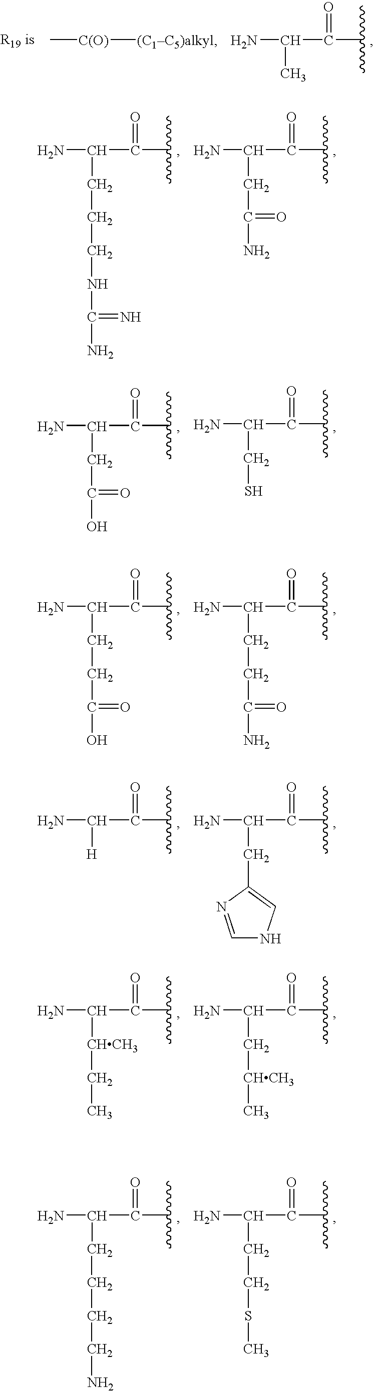 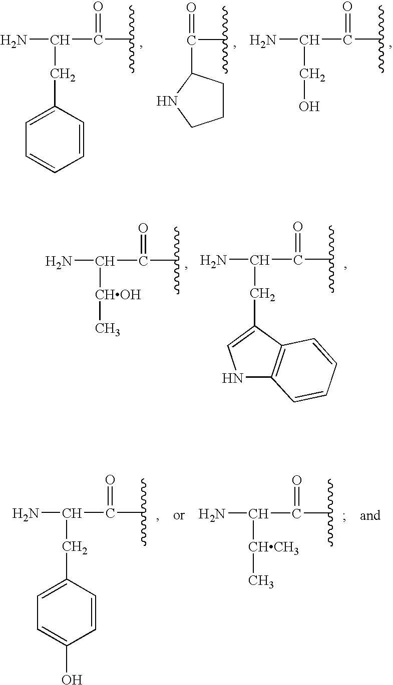
the stereochemical configuration at any stereocenter of a compound represented by 4 is R, S, or a mixture of these configurations.

Another aspect of the present invention relates to a compound represented by formula 5: 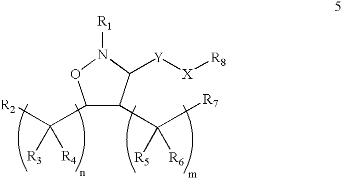
or pharmaceutically acceptable salts, solvates, or hydrates thereof,
wherein
Y is C(R)₂ or —C(O)—;
R represents independently for each occurrence H, alkyl, cycloalkyl, aralkyl, aryl or heteroaryl;
X is NR₁₀;
m is 0, 1, 2, 3, 4, 5, or 6;
n represents independently for each occurrence 0, 1, 2, 3, 4, 5, or 6;
R₁ is alkyl, aralkyl, —(C(R)₂)_{q}-cycloalkyl, or has the formula 5b: 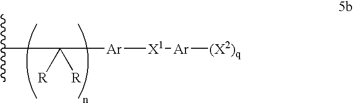
   or has the formula 5c: 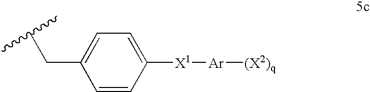
   wherein
   X¹ is a bond, O, alkyl, alkenyl, or alkynyl;
   X² is H, halide, hydroxyl, alkyl, fluoroalkyl, —C(O)R₁₈, —CO₂R₁₈, —O-heterocycloalkyl, or —O-aryl; and
   R₁₈ represents independently for each occurrence H or alkyl;
Ar represents independently for each occurrence a monocyclic aryl with 6-14 ring atoms; or a bicyclic heteroaryl with 5-14 ring atoms, of which one, two or three ring atoms are 0;
q represents independently for each occurrence 1, 2, 3, 4, or 5;
R₂ and R₇ represent independently H, hydroxyl, —N(R₁₁)₂, —OP(O)(OR₁₂)₂, or —OC(O)(C(R)₂)_{q}N(R₉)₂; or R₂ and R₇ taken together form an optionally substituted heteroalkyl linkage containing 1 to 6 carbon atoms;
R₃ and Rr represent independently for each occurrence H;
R₄ and R₅ represent independently for each occurrence H or alkyl;
R₉ and R₁₀ represent independently for each occurrence H or alkyl;
R₈ has the formula 5d: 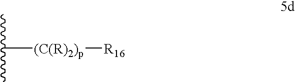
wherein
   p is 2 or 3; and
   R₁₆ is heterocycloalkyl, alkoxyl, heteroaryl, N(R)CO₂-alkyl, or C(O)N(R₉)aryl; and
the stereochemical configuration at any stereocenter of a compound represented by 5 is R, S, or a mixture of these configurations.

In certain embodiments, the present invention relates to the aforementioned compound, wherein R₁ has the formula 5b: 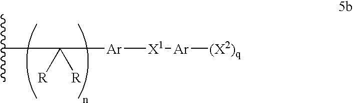
wherein
   X¹ is a bond, O, alkyl, alkenyl, or alkynyl;
   X² is H, halide, hydroxyl, alkyl, fluoroalkyl, —C(O)R₁₈, —CO₂R₁₈, —O-heterocycloalkyl, or —O-aryl; and
   R₁₈ represents independently for each occurrence H or alkyl.

In certain embodiments, the present invention relates to the aforementioned compound, wherein R₁ has the formula 5b: 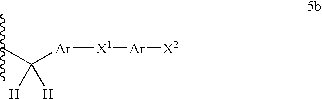
wherein
   X¹ is a bond, alkyl, —CH═CH—, or —C≡C—; and
   X² is H, halide, hydroxyl, alkyl, —CF₃, —C(O)alkyl, —CO₂H, —O-heterocycloalkyl, or —O-aryl.

In certain embodiments, the present invention relates to the aforementioned compound, wherein R₁ has the formula 5b: 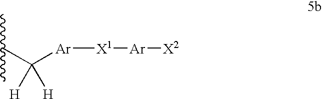
wherein
   X¹ is a bond, alkyl, —CH═CH—, or —C≡C—; and
   X² is H, chloride, hydroxyl, methyl, t-butyl, —C(O)alkyl, —CO₂H, —O-heterocycloalkyl, or —O-phenyl; and
   Ar is phenyl.

In certain embodiments, the present invention relates to the aforementioned compound, wherein R₁ is alkyl or aralkyl.

In certain embodiments, the present invention relates to the aforementioned compound, wherein R₂ and R₇ are hydroxyl; R₃, R₅, and R₆ are H; R₄ is alkyl; and m and n are 1.

In certain embodiments, the present invention relates to the aforementioned compound, wherein R₂ is hydroxyl; R₃, R₅, and R₆ are H; R₄ is alkyl; R₇ is alkylamino; and m and n are 1.

In certain embodiments, the present invention relates to the aforementioned compound, wherein R₂ is hydroxyl, —N(R₁₁)₂, or —OP(O)(OR₁₂)₂; R₃, R₄, and R₇ are H; n is 1; m is 0; R₁₁ is H; and R₁₂ is H.

In certain embodiments, the present invention relates to the aforementioned compound, wherein R₂, R₅, and R₆, are H; n is 0; m is 1; and R₇ is hydroxyl.

In certain embodiments, the present invention relates to the aforementioned compound, wherein R₈ has the formula 5c: 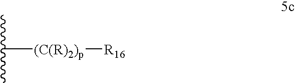
wherein
   p is 2 or 3; and
   R₁₆ is heteroaryl.

In certain embodiments, the present invention relates to the aforementioned compound, wherein R₈ has the formula 5c: 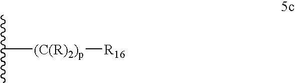
wherein
   p is 2 or 3; and
   R₁₆ is selected from the group consisting of

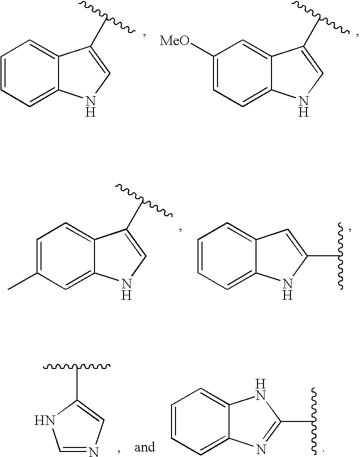

In certain embodiments, the present invention relates to the aforementioned compound, wherein R₈ has the formula 5c: 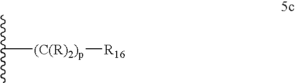
wherein
p is 2 or 3; and
R₁₆ is

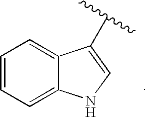

In certain embodiments, the present invention relates to the aforementioned compound, wherein R₈ has the formula 5c: 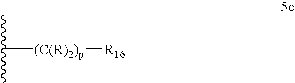
wherein independently for each occurrence:
p is 2 or 3; and
R₁₆ is OEt, —N(H)CO₂-alkyl, or

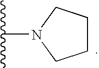

In certain embodiments, the present invention relates to the aforementioned compound, wherein R₁ has the formula 5b: 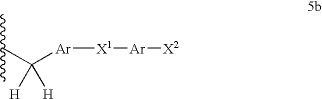
wherein
X¹ is a bond, alkyl, —CH═CH—, or —C≡C—; and
X² is H, chloride, hydroxyl, methyl, t-butyl, —C(O)alkyl, —CO₂H, —O-heterocycloalkyl, or —O-phenyl; and
Ar is phenyl; and
R₈ has the formula 5c: 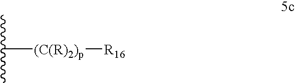
wherein
   p is 2 or 3; and
   R₁₆ is selected from the group consisting of OEt, —N(H)CO₂-alkyl,

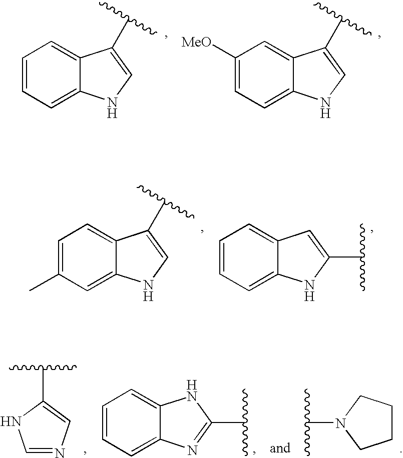

In certain embodiments, the present invention relates to the aforementioned compound, wherein R₁ has the formula 5b: 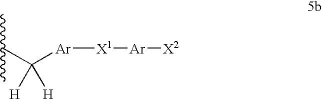
wherein
   X¹ is a bond, alkyl, —CH═CH—, or —C≡C—; and
   X² is H, chloride, hydroxyl, methyl, t-butyl, —C(O)alkyl, —CO₂H, —O-heterocycloalkyl, or —O-phenyl; and
   Ar is phenyl; and
R₈ has the formula 5c: 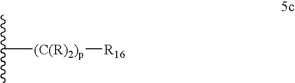
wherein
   p is 2 or 3; and
   R₁₆ is selected from the group consisting of

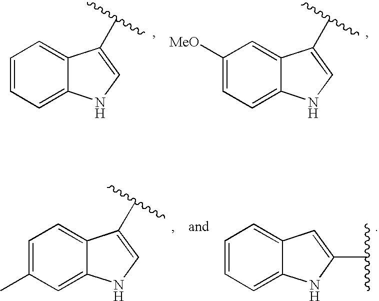

Another aspect of the present invention relates to a compound selected from the group consisting of

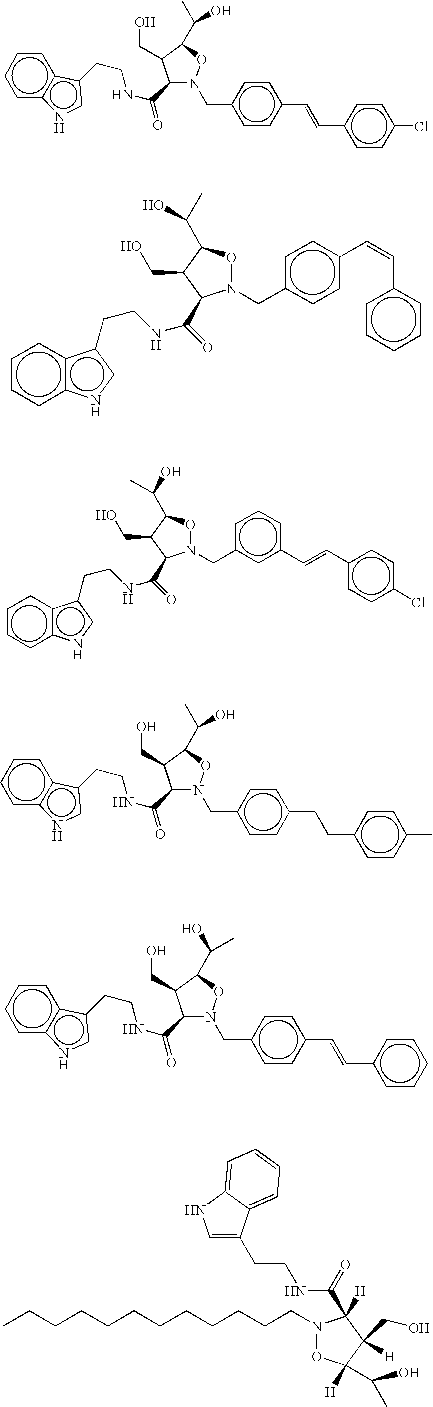

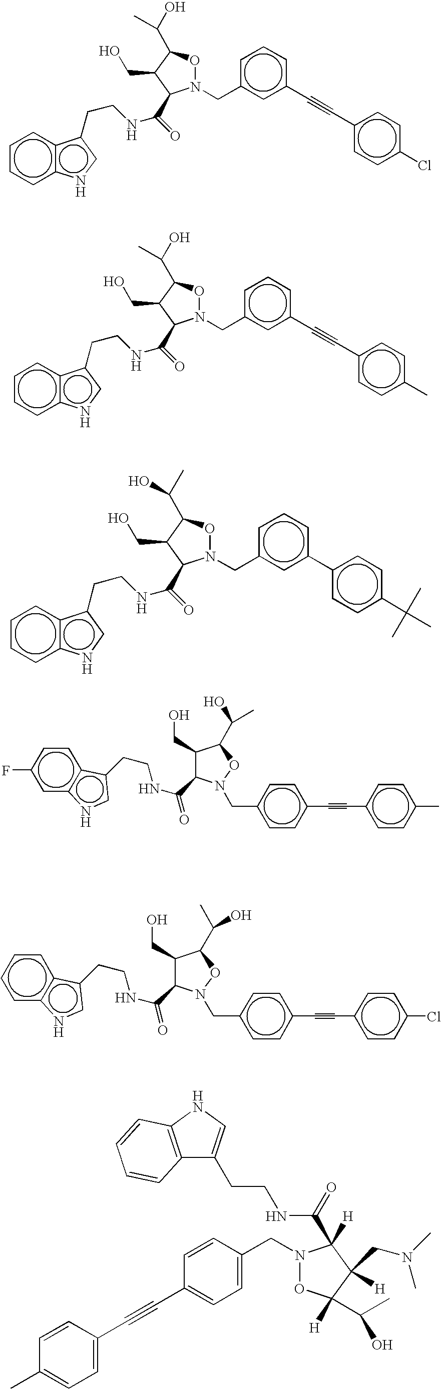

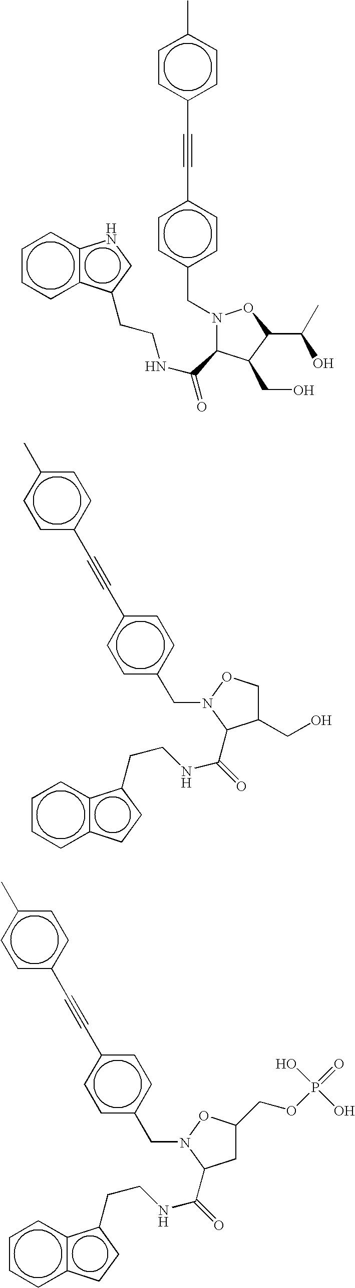

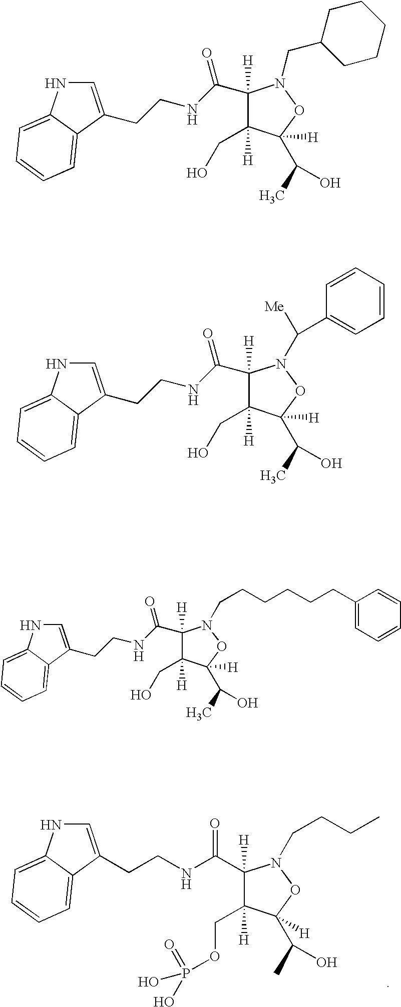

Another aspect of the present invention relates to a pharmaceutical composition comprising a compound of formula 1, 2, 3, 4, or 5 as described above and at least one pharmaceutically acceptable excipient.

Methods of the Invention

One aspect of the present invention relates to a method for inhibiting bacterial cell growth comprising contacting a bacterial cell with a compound represented in the general formula 1, 2, 3, 4, or 5 as described above.

In certain embodiments, the present invention relates to the aforementioned method, wherein said bacteria is a gram-positive bacteria.

In certain embodiments, the present invention relates to the aforementioned method, wherein said bacteria is a gram-negative bacteria.

In certain embodiments, the present invention relates to the aforementioned method, wherein said bacteria is selected from the group consisting of Staphylococcus, Streptococcus, Enterococcus, Moraxella, Haemophilus, Mycobacteria, Neisseria, Micrococcus, Peptococcus, Peptostreptococcus, Bacillus, Clostridium, Lactobacillus, Listeria, Erysipelothrix, Propionibacterium, Eubacterium, and Corynebacterium.

In certain embodiments, the present invention relates to the aforementioned method, wherein said bacteria is selected from the group consisting of Staphylococcus, Streptococcus, Enterococcus, Moraxella, and Haemophilus.

In certain embodiments, the present invention relates to the aforementioned method, wherein said bacteria is selected from the group consisting of Staphylococcus, Streptococcus, and Enterococcus.

In certain embodiments, the present invention relates to the aforementioned method, wherein said bacteria is pneumococci, penicillin resistant Streptococcus pneumoniae, multiply resistant Enterococcus faecium, or methicillin resistant strains of Staphylococcus aureus or coagulase negative Staphylococci.

In certain embodiments, the present invention relates to the aforementioned method, wherein said bacteria is Staphylococcus aureus, Staphylococcus epidermidis, Methicillin-resistant S. aureus, Vancomycin-intermediate S. aureus, Methicillin-resistant S. epidermidis, Streptococcus pneumoniae, Penicillin-resistant Streptococcus pneumoniae, Multi-drug resistant Penicillin-resistant Streptococcus pneumoniae, Streptococcus pyogenes, Enterococcus faecalis, Vancomycin-intermediate E. faecalis, Vancomycin-resistant E. faecalis, Enterococcus faecium, Vancomycin-intermediate E. faecium, Moraxella catarrahalis, Haemophilus influenzae, E. coli, or Neisseria gonorrhoeae.

In certain embodiments, the present invention relates to the aforementioned method, wherein said bacteria is Staphylococcus aureus, Staphylococcus epidermidis, Methicillin-resistant S. aureus, Vancomycin-intermediate S. aureus, Methicillin-resistant S. epidermidis, Streptococcus pneumoniae, Penicillin-resistant Streptococcus pneumoniae, Multi-drug resistant Penicillin-resistant Streptococcus pneumoniae, Streptococcus pyogenes, Enterococcus faecalis, Vancomycin-intermediate E. faecalis, Vancomycin-resistant E. faecalis, Enterococcus faecium, or Vancomycin-intermediate E. faecium.

In certain embodiments, the present invention relates to the aforementioned method, wherein said bacteria is Staphylococcus aureus, Methicillin-resistant S. aureus, Streptococcus pneumoniae, Penicillin-resistant Streptococcus pneumoniae, Multi-drug resistant Penicillin-resistant Streptococcus pneumoniae, Enterococcus faecalis, Vancomycin-intermediate E. faecalis, or Vancomycin-resistant E. faecalis.

In certain embodiments, the present invention relates to the aforementioned method, wherein said bacteria is Staphylococcus aureus or Methicillin-resistant S. aureus.

In certain embodiments, the present invention relates to the aforementioned method, wherein the bacteria is contacted with the compound in vitro.

In certain embodiments, the present invention relates to the aforementioned method, wherein the bacteria is contacted with the compound in vivo.

In certain embodiments, the present invention relates to the aforementioned method, wherein the compound is administered to an animal suffering from, or at risk of developing, bacteremia, a skin/wound infection, a lower respiratory infection, endocarditis, or infection of the urinary tract.

In certain embodiments, the present invention relates to the aforementioned method, wherein the compound is administered parenterally.

In certain embodiments, the present invention relates to the aforementioned method, wherein the compound is administered intramuscularly, intravenously, subcutaneously, orally, topically or intranasally.

In certain embodiments, the present invention relates to the aforementioned method, wherein the compound is administered systemically.

In certain embodiments, the present invention relates to the aforementioned method, wherein the compound is administered to a mammal.

In certain embodiments, the present invention relates to the aforementioned method, wherein the compound is administered to a primate.

In certain embodiments, the present invention relates to the aforementioned method, wherein the compound is administered to a human.

In certain embodiments, the present invention relates to the aforementioned method, wherein said compound is the compound of formula 5 as described above.

Pharmaceutical Compositions

In another aspect, the present invention provides pharmaceutically acceptable compositions which comprise a therapeutically-effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As set out above, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, oral, intravenous, intracerebroventricular and subcutaneous doses of the compounds of this invention for a patient, when used for the indicated analgesic effects, will range from about 0.0001 to about 100 milligrams per kilogram of body weight per day.

If desired, the effective daily dose of the active compound may be administered as two, three, four, five, six or more sub-doses administered separately at appropriate intervals throughout the day, optionally, in unit dosage forms. Preferred dosing is one administration per day.

While it is possible for a compound of the present invention to be administered alone, it is preferable to administer the compound as a pharmaceutical formulation (composition).

The compounds according to the invention may be formulated for administration in any convenient way for use in human or veterinary medicine, by analogy with other pharmaceuticals.

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin, lungs, or mucous membranes; or (4) intravaginally or intrarectally, for example, as a pessary, cream or foam; (5) sublingually or buccally; (6) ocularly; (7) transdermally; or (8) nasally.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable carriers and can also be administered in conjunction with antimicrobial agents such as penicillins, cephalosporins, aminoglycosides and glycopeptides. Conjunctive therapy, thus includes sequential, simultaneous and separate administration of the active compound in a way that the therapeutic effects of the first administered one is not entirely disappeared when the subsequent is administered.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” 0 and B books, Corvallis, Ore., U.S.A., 1977).

Micelles

Recently, the pharmaceutical industry introduced microemulsification technology to improve bioavailability of some lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other things, microemulsification provides enhanced bioavailability by preferentially directing a bsorption to the lymphatic system instead of the circulatory system, which thereby bypasses the liver, and prevents destruction of the compounds in the hepatobiliary circulation.

In one aspect of invention, the formulations contain micelles formed from a compound of the present invention and at least one amphiphilic carrier, in which the micelles have an average diameter of less than about 100 nm. More preferred embodiments provide micelles having an average diameter less than about 50 nm, and even more preferred embodiments provide micelles having an average diameter less than about 30 nm, or even less than about 20 nm.

While all suitable amphiphilic carriers are contemplated, the presently preferred carriers are generally those that have Generally-Recognized-as-Safe (GRAS) status, and that can both solubilize the compound of the present invention and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Particularly preferred amphiphilic carriers are saturated and monounsaturated polyethyleneglycolyzed fatty acid glycerides, such as those obtained from fully or partially hydrogenated various vegetable oils. Such oils may advantageously consist of tri-. di- and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers are particularly contemplated, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc (produced and distributed by a number of companies in USA and worldwide).

Polymers

Hydrophilic polymers suitable for use in the present invention are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. Preferred polymers are those having a molecular weight of from about 100 or 120 daltons up to about 5,000 or 10,000 daltons, and more preferably from about 300 daltons to about 5,000 daltons. In a particularly preferred embodiment, the polymer is polyethyleneglycol having a molecular weight of from about 100 to about 5,000 daltons, and more preferably having a molecular weight of from about 300 to about 5,000 daltons. In a particularly preferred embodiment, the polymer is polyethyleneglycol of 750 daltons (PEG(750)). Polymers may also be defined by the number of monomers therein; a preferred embodiment of the present invention utilizes polymers of at least about three monomers, such PEG polymers consisting of three monomers (approximately 150 daltons).

Other hydrophilic polymers which may be suitable for use in the present invention include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a formulation of the present invention comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

Cyclodextrins

Cyclodextrins are cyclic oligosaccharides, consisting of 6, 7 or 8 glucose units, designated by the Greek letter .alpha., beta. or .gamma., respectively. Cyclodextrins with fewer than six glucose units are not known to exist. The glucose units are linked by alpha-1,4-glucosidic bonds. As a consequence of the chair conformation of the sugar units, all secondary hydroxyl groups (at C-2, C-3) are located on one side of the ring, while all the primary hydroxyl groups at C-6 are situated on the other side. As a result, the external faces are hydrophilic, making the cyclodextrins water-soluble. In contrast, the cavities of the cyclodextrins are hydrophobic, since they are lined by the hydrogen of atoms C-3 and C-5, and by ether-like oxygens. These matrices allow complexation with a variety of relatively hydrophobic compounds, including, for instance, steroid compounds such as 17.beta.-estradiol (see, e.g., van Uden et al. Plant Cell Tiss. Org. Cult. 38:1-3-113 (1994)). The complexation takes place by Van der Waals interactions and by hydrogen bond formation. For a general review of the chemistry of cyclodextrins, see, Wenz, Agnew. Chem. Int. Ed. Engl., 33:803-822 (1994).

The physico-chemical properties of the cyclodextrin derivatives depend strongly on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility.

Numerous cyclodextrins and methods for their preparation have been described. For example, Parmeter (I), et al. (U.S. Pat. No. 3,453,259) and Gramera, et al. (U.S. Pat. No. 3,459,731) described electroneutral cyclodextrins. Other derivatives include cyclodextrins with cationic properties [Parmeter (II), U.S. Pat. No. 3,453,257], insoluble cross-linked cyclodextrins (Solms, U.S. Pat. No. 3,420,788), and cyclodextrins with anionic properties [Parmeter (III), U.S. Pat. No. 3,426,011]. Among the cyclodextrin derivatives with anionic properties, carboxylic acids, phosphorous acids, phosphinous acids, phosphonic acids, phosphoric acids, thiophosphonic acids, thiosulphinic acids, and sulfonic acids have been appended to the parent cyclodextrin [see, Parmeter (III), supra]. Furthermore, sulfoalkyl ether cyclodextrin derivatives have been described by Stella, et al. (U.S. Pat. No. 5,134,127).

Liposomes

Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 μm in diameter; large unilamellar vesicles (LUVS) are typically larger than 0.05 μm. Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles.

One aspect of the present invention relates to formulations comprising liposomes containing a compound of the present invention, where the liposome membrane is formulated to provide a liposome with increased carrying capacity. Alternatively or in addition, the compound of the present invention may be contained within, or adsorbed onto, the liposome bilayer of the liposome. The compound of the present invention may be aggregated with a lipid surfactant and carried within the liposome's internal space; in these cases, the liposome membrane is formulated to resist the disruptive effects of the active agent-surfactant aggregate.

According to one embodiment of the present invention, the lipid bilayer of a liposome contains lipids derivatized with polyethylene glycol (PEG), such that the PEG chains extend from the inner surface of the lipid bilayer into the interior space encapsulated by the liposome, and extend from the exterior of the lipid bilayer into the surrounding environment.

Active agents contained within liposomes of the present invention are in solubilized form. Aggregates of surfactant and active agent (such as emulsions or micelles containing the active agent of interest) may be entrapped within the interior space of liposomes according to the present invention. A surfactant acts to disperse and solubilize the active agent, and may be selected from any suitable aliphatic, cycloaliphatic or aromatic surfactant, including but not limited to biocompatible lysophosphatidylcholines (LPCs) of varying chain lengths (for example, from about C.sub.14 to about C.sub.20). Polymer-derivatized lipids such as PEG-lipids may also be utilized for micelle formation as they will act to inhibit micelle/membrane fusion, and as the addition of a polymer to surfactant molecules decreases the CMC of the surfactant and aids in micelle formation. Preferred are surfactants with CMCs in the micromolar range; higher CMC surfactants may be utilized to prepare micelles entrapped within liposomes of the present invention, however, micelle surfactant monomers could affect liposome bilayer stability and would be a factor in designing a liposome of a desired stability.

Liposomes according to the present invention may be prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic DD, Liposomes from physics to applications, Elsevier Science Publishers BV, Amsterdam, 1993.

For example, liposomes of the present invention may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, the active agent is first dispersed by sonication in a lysophosphatidylcholine or other low CMC surfactant (including polymer grafted lipids) that readily solubilizes hydrophobic molecules. The resulting micellar suspension of active agent is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In one aspect of the present invention, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323 (Apr. 12, 1988).

Release Modifiers

The release characteristics of a formulation of the present invention depend on the encapsulating material, the concentration of encapsulated drug, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In all cases the amount should be between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween® and Pluronic®. Pore forming agents which a dd microstructure to the matrices (i.e., water soluble compounds such as inorganic salts and sugars) are added as particulates. The range should be between one and thirty percent (w/w polymer).

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Synthesis of Benzyl Hydroxyamines

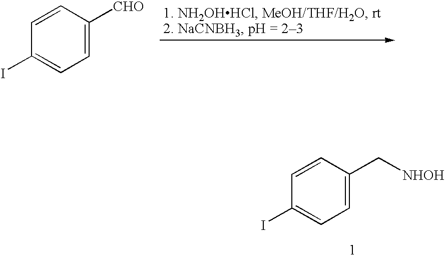

To a solution of 4-iodobenzaldehyde (0.025 mmol) in MeOH/THF (40 mL, 3:1) was added aqueous solution of NH₂OH—HCl (10 mL). The pH was adjusted to 9 using of 6 N KOH. The reaction was stirred at rt for 2 h and NaCNBH₃ (1.5 g, 0.025 mmol) was added followed by a crystal of methyl orange. The solution was acidified to a pH 2 and the resulting ruby red color was maintained for the duration of the reaction by the addition of 1 N HCl. After 2 h, another portion of NaCNBH₃ (1.5 g, 0.025 mmol) was added. The mixture was stirred for 14 h, at which point, ⅔ of solvent was evaporated and the pH was raised to 9-10 by addition of a 6 N KOH aqueous solution. This mixture was extracted with CH₂Cl₂ (3×100 mL). The organic layers were combined, washed with water then brine. The organic layer was dried (MgSO₄), filtered and evaporated in vacuo to afford 1 (5.7 g, 91%) an off-white solid.

### Example 2

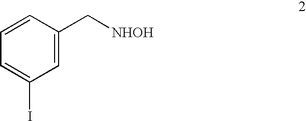

Hydroxyamine 2 was synthesized according to the procedure described in Example 1 using 3-iodobenzaldehyde in place of 4-iodobenzaldehyde affording a 90% yield of the desired product.

### Example 3

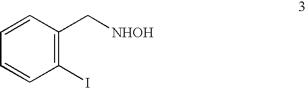

Hydroxyamine 3 was synthesized according to the procedure described in Example 1 using 2-iodobenzaldehyde in place of 4-iodobenzaldehyde affording a 85% yield of the desired product.

### Example 4

### Synthesis of Nitrone Acids for use in [3+2] Cycloadditions

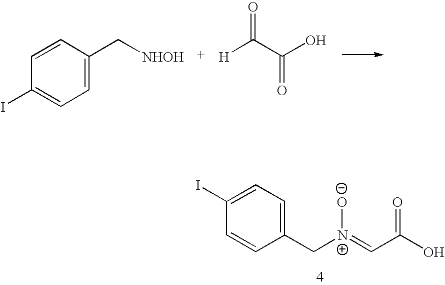

(Keirs, D.; Overton, K. Heterocycles 1989, 28, 841-848) To a suspension of N-(4-iodobenzyl)hydroxylamine (10 g, 42.5 mmol) in CH₂Cl₂ (200 mL) under nitrogen was added glyoxylic acid monohydrate (4.7 g, 51.1 mmol). The reaction mixture was stirred for 24 h at rt. The reaction mixture was washed with water (2×200 mL), brine, dried (MgSO₄), filtered and concentrated in vacuo Et₂O (50 mL) was added to the yellowish solid and the suspension was titrated and filtered to afford 4 as a cream colored solid. The mother liquor was concentrated in vacuo and the solid was washed with Et₂O and then filtered to afford 4 (11.8 g, 93% yield).

### Example 5

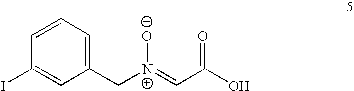

Nitrone acid 5 was synthesized according to the procedure described in Example 4 using N-(3-iodobenzyl)hydroxylamine in place of N-(4-iodobenzyl)hydroxylamine affording a 90% yield of the desired product.

### Example 6

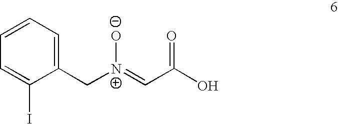

Nitrone acid 6 was synthesized according to the procedure described in Example 4 using N-(2-iodobenzyl)hydroxylamine in place of N-(4-iodobenzyl)hydroxylamine affording a 90% yield of the desired product.

### Example 7

### Synthesis of Nitrone Carboxylic Methyl Esters for use in [3+2] Cycloadditions

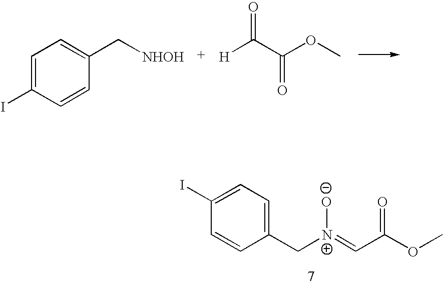

To a solution of N-(4-iodobenzyl)hydroxylamine (16 g, 64 mmol) in benzene (320 mL) was added the methyl glyoxylate (6.8 g, 80 mmol). The mixture was heated to 120° C. for 3 h using a Dean Stark trap. The solution was cooled to rt and the solvent was concentrated in vacuo to give 7 (19.1 g, 93%) as a yellow solid.

### Example 8

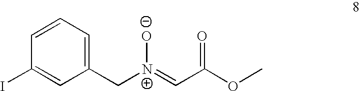

Nitrone methyl ester 8 was synthesized according to the procedure described in Example 7 using N-(3-iodobenzyl)hydroxylamine in place of N-(4-iodobenzyl)hydroxylamine affording a 85% yield of the desired product.

### Example 9

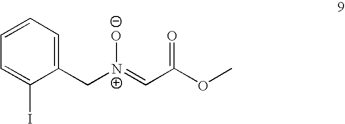

Nitrone methyl ester 9 was synthesized according to the procedure described in Example 7 using N-(2-iodobenzyl)hydroxylamine in place of N-(4-iodobenzyl) hydroxylamine affording a 90% yield of the desired product.

### Example 10

### Synthesis of Dipolarophiles

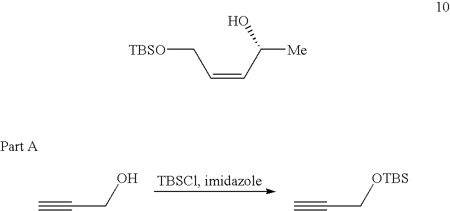

To a solution of TBSCl (215 g, 1.43 mol) in CH₂Cl₂ (1.2 L) at 0° C. was added imidazole (97 g, 1.43 mol). Propargylic alcohol (83 mL, 1.43 mol) was added dropwise and the suspension was allowed to warm to rt and stirred for 60 min. The reaction was quenched by the addition of water (500 mL). The mixture was concentrated in vacuo and the residue was extracted with hexanes (3×500 mL). The organic extracts were combined and washed with brine, dried (MgSO₄), filtered and concentrated in vacuo to afford an oil which was purified by distillation (70° C./˜10 mm Hg) afforded the desired product (179 g, 74%).

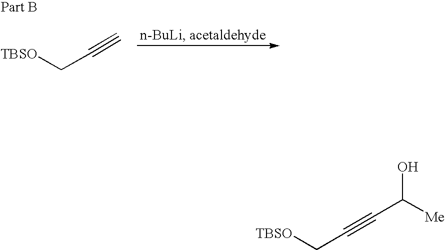

To a solution of tert-Butyldimethyl(2-propynyloxy)silane (freshly distilled, 27 g, 0.16 mol) in THF (300 mL) at −78° C. was added n-BuLi (119 mL of 1.6 M in hexanes, 0.19 mol). After 15 min. acetaldehyde (10 mL, 0.19 mol.) was added. The reaction mixture was stirred for 30 min. and quenched with an aqueous solution of 5% (m/v) NH₄Cl (50 mL) and water (100 mL). A half volume of THF was evaporated in vacuo and the mixture was poured into water (150 mL). This mixture was extracted with hexanes (3×200 mL) and Et₂O (100 mL). The combined extracts were dried (Mg₂SO₄) and concentrated in vacuo. The crude product was purified by distillation (115° C./1 mm Hg (bath temp. 155° C.) to afford the product (30 g, 88%).

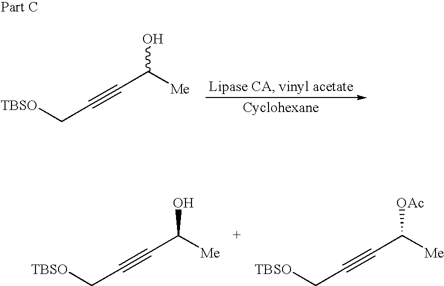

Lipase CA (Candida Antarctica immobilized on macropous acryl resin, Sigma L-4777 Lot 11K127) (1 g) was added to a mixture of the propargyl alcohol (10 g, 0.47 mol) and vinyl acetate (129 mL, 0.14 mol) in cyclohexane (380 mL). The reaction mixture was stirred for 48 h, then filtered and the resin was rinsed with EtOAc (50 mL). The filtrate and the rinses were combined and concentrated in vacuo. The crude material was purified by column chromatography (hexane/EtOAc, 95:5 to 80:20) to give 5.3 g of the alcohol and 6.5 g of the acetate (93% ee).

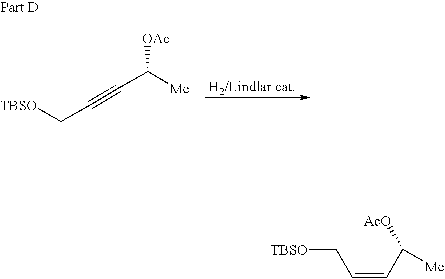

To a stirred solution of the propargyl acetate (60 g, 0.23 mmol) in EtOAc (700 mL) was added quinoline (30 mL) and Lindlar cat. (6 g) and placed under an atmosphere of H₂. After 9 h the reaction mixture was filtered and the filtrate was concentrated in vacuo. The crude was purified by column chromatography (hexane/EtOAc 95:5) to afford of the desired product (57.5 g, 97%).

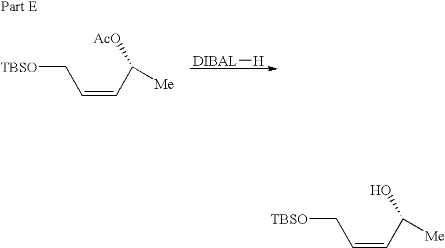

To a stirred solution of the allylic acetate (50 g, 0.19 mol) in CH₂Cl₂ (400 mL) at −78° C. was added DIBAL-H (426 mL of a 1 M solution in heptane, 0.43 mol). After 15 min. the reaction was diluted with Et₂O (400 mL) and quenched with brine (150 mL). The reaction mixture was warmed to rt and stirred for an additional 2 h. The reaction mixture was dried (MgSO₄), filtered and concentrated in vacuo. The crude mixture was purified by column chromatography (hexane/EtOAc, 80:20) to afford (41 g) of the desired product.

### Example 11

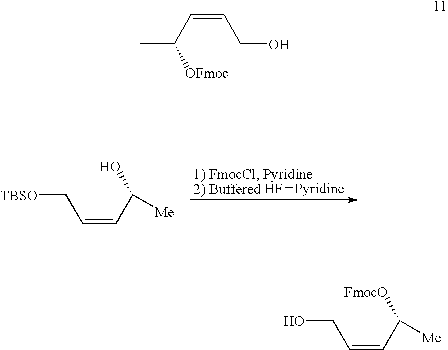

To a stirred solution of the allylic alcohol (40 g, 0.19 mol) in pyridine (400 mL) and CH₂Cl₂ (100 mL) at 0° C. was added Fmoc-Cl (62 g). The reaction was warmed to rt, stirred for an additional 30 min, and quenched with water (500 mL). The layers were separated and the water phase was extracted with Et₂O (2×200 mL) and the combined extracts were washed with CuSO₄ (aq) water, brine, dried (MgSO4), filtered and concentrated in vacuo to give Fmoc-protected alcohol. The crude material was carried on to the next step without purification.

The crude product (35 g) was placed in a plastic bottle and THF (100 mL) was added followed by a solution of HF-pyridine (HF-Py (7.7 mL)/pyridine (15.4 mL)/THF (76.9 mL). The reaction mixture was kept at rt for 4 h and treated with TMSOMe for 60 min and poured into water (500 mL). The layers were separated and the aqueous phase was extracted with Et₂O (3×200 mL). The organic extracts were collected and washed with aqueous NaHCO₃, brine, dried (MgSO4), filtered and concentrated in vacuo to give the crude allylic alcohol. The crude material was purified by column chromatography to afford 14.4 g of the desired product.

### Example 12

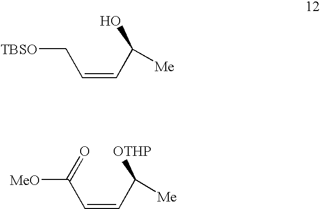 Part A

A solution of bis (2,2,2-trifluoroethyl)phosphonoacetic acid methyl ester (28 g, 0.1 mmol) and 18-crown-6 (132 g, 0.50 mmol) in THF (2 L) was cooled to −78° C. under nitrogen. To the cooled solution was added a 0.6 M solution of potassium bis(trimethylsilyl)amide in toluene (20 g, 0.1 mmol). (S)-2-(tetrahydropyranyloxy) propanal (synthesis described in J. Chem. Soc., Perkin. Trans. 1, 1994, 2791) (16 g, 0.1 mmol) was then added and the resulting mixture was stirred for 30 min. at −78° C. Saturated ammonium chloride was then added and the product was extracted with Et₂O (3×500 mL). The ether extracts were combined, dried over sodium sulfate, filtered and concentrated in vacuo. The crude material was purified via silica gel chromatography to yield 13.5 g of the product.

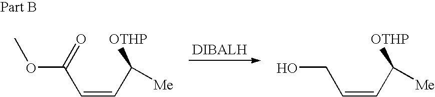

4(S)-(Tetrahydro-pyran-2-yloxy)-pent-2-enoic acid methyl ester (10 g, 46.7 mmol) was reduced with DIBAL-H according to the procedure described in J. Chem. Soc., Perkin. Trans. 1 1994, 2791 to yield 4(S)-(Tetrahydro-pyran-2-yloxy)-pent-2-en-1-ol in 88% yield.

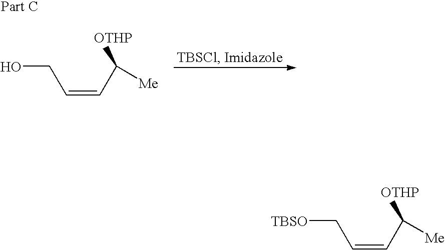

To a solution of 4(S)-(tetrahydro-pyran-2-yloxy)-pent-2-en-1-ol (4.0 g, 22 mmol) in THF (20 mL) was added imidazole (slowly) (3.66 g, 53.5 mmol) followed by TBSCl (1.2 eq., 3.89 g, 25.8 mmol). The reaction mixture was stirred at ambient temperature for 4 h, quenched with water (20 mL) and extracted with Et₂O (3×10 mL). The combined organic extracts were washed with water (5×50 mL), brine (1×50 mL), dried (MgSO₄), filtered and concentrated in vacuo. The oil was purified by column chromatography (2:1 Hexane/EtOAc) to give the desired TBDMS ether (5.9 g, 92%) of a colorless oil.

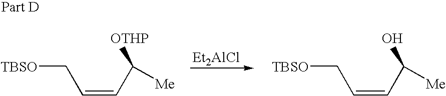

The THP protecting group was removed from t-butyl-dimethyl-[4(S)-(tetrahydro-pyran-2-yloxy)-pent-2-enyloxy]-silane (10 g, 33 mmol) according to the procedure described in Tetrahedron Letters 1984, 25, 663 to afford the product in 83% yield.

### Example 13

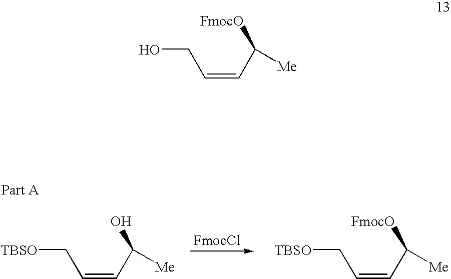

To a solution of 5-(tert-butyl-dimethyl-silanyloxy)-pent-3-en-2(S)-ol (3.95 g, 18.3 mmol) in pyridine (20 mL) was added FmocCl (6.14 g, 23.7 mmol, 1.3 eq.). The reaction mixture was stirred overnight at rt. The reaction mixture was slowly quenched with water (20 mL) and extracted with Et₂O (3×15 mL). The combined organic extracts were washed with water (3×50 mL), 5% KH₂PO₄ (3×50 mL), and brine (1×50 mL), dried over MgSO₄ and filtered. Concentration and column chromatography afforded 6.98 g (87%) of a pale yellow oil.

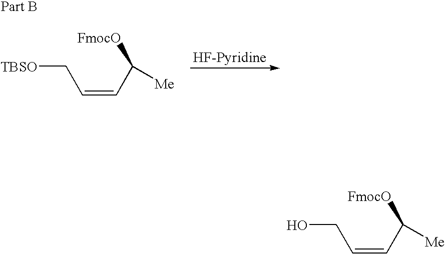

To a solution of carbonic acid 4-(tert-butyl-dimethyl-silanyloxy)-1-methyl-butyl-2(S)-enyl ester 9H-fluoren-9-yl methyl ester (700 mg, 1.60 mmol) in Et₂O (5 mL) in a plastic Wharton® tube was added slowly in 6 portions HF/pyridine (70% HF in pyridine, 6 mL). The reaction was monitored by TLC. When the reaction was complete the mixture was cooled to 0° C. with an ice bath and then quenched with TMSOMe (10 mL). The reaction mixture was stirred for 30 min. while warming to ambient temperature. The reaction mixture was poured into water (50 mL), the layers were separated and the aqueous layer was extracted with Et₂O (3×15 mL). The combined organic extracts were washed with water (3×30 mL), 5% KH₂PO₄ (3×30 mL), brine (1×30 mL), dried (Na₂SO₄) and filtered. Concentration and column chromatography gave 471 mg (91%) of a clear colorless oil.

### Example 14

### General Procedures for [3+2] Cycloadditions

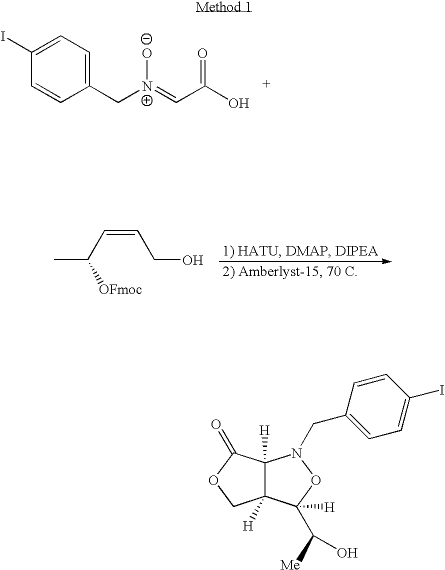

To a solution of nitronecarboxylic acid 4 (1.4 g, 3.1 mmol) in CH₂Cl₂ was added allylic alcohol 11 (1.0 g, 3.1 mmol), HATU (2.0 g, 6 mmol) and DMAP (0.56 g, 4.6 mmol). The solution was cooled in an ice-bath and stirred for 1.0 h. Diisopropylethylamine (0.44 g, 0.6 mL, 4.6 mmol) was added drop-wise over 15 min and the reaction was stirred at 0° C. for 1 h. The solution was diluted with CH₂Cl₂-5% NaHCO₃ (300 mL, 1:1) and the aqueous layer was extracted with CH₂Cl₂ (2×125 mL). The combined organic extracts were combined and washed with water (200 mL), dried (MgSO₄), filtered and concentrated in vacuo to afford an residue. The residue was suspended in THF (10 mL), diisopropylethylamine (0.12 g, 0.167 mL, 1 mmol) was added and the solution was heated at reflux for 1 h, cooled to rt and concentrated in vacuo. The residue was taken up in THF (8 mL), 1.33 g of Amberlyst-15 was added and the mixture was heated at 70° C. for 2 h. Again the solution was cooled, filtered and concentrated in vacuo. The crude material was purified by flash chromatography to yield the desired product in 5.9% yield.

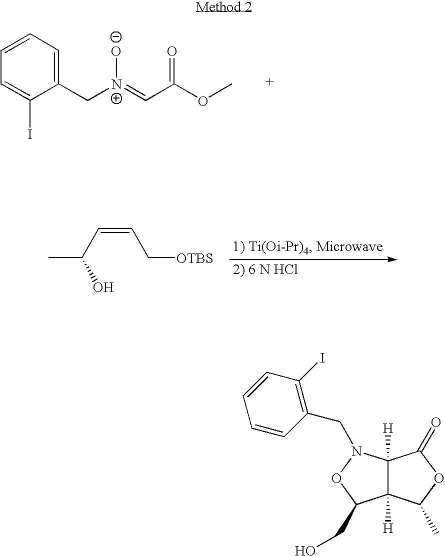

To a solution of nitrone methyl ester 9 (8.1 g, 38 mmol) and secondary alcohol 10 (12 g, 38 mmol) in toluene (40 mL) was added Ti(OCH(CH₃)₂)₄ (16 g, 17 mL, 56 mmol). The suspension was heated in microwave oven to 140° C. for 30 min, and allowed to cool to rt. The solution was diluted with EtOAc (150 mL) and 3-(dimethylamino)-1,2-propanediol (7 g, 7 mL, 58 mmol) and stirred at rt for 8 h. To the solution was added water (100 mL) and the organic phase was separated, the aqueous was washed with EtOAc (3×30 mL). The combined organic extracts were washed with water (100 mL), brine (100 mL), dried (Na₂SO₄), filtered and concentrated in vacuo. The crude material was purified by flash chromatography (Et₂O/CH₂Cl₂, 1:29) to afford the lactone (13.5 g, 71%).

To a solution of isoxazolidine (13.5 g, 26 mmol) in THF (120 mL) was added 6 N HCl (67 mL). The solution was stirred at rt for 1.5 h, diluted water (25 mL) and extracted with EtOAc (3×80 mL), the organic extracts were combined and washed with saturated NaHCO₃ (50 mL), brine (50 mL), dried (Na₂SO₄) and concentrated in vacuo. The crude material was purified by chromatography on silica a gel (mesh 230-400) (Et₂O—CH₂Cl₂, a gradient of 1:4 to 1:2) to give desired product (9.5 g, 64% overall yield for 2 steps).

### Example 15

### Synthesis of Isoxazolidine Cores

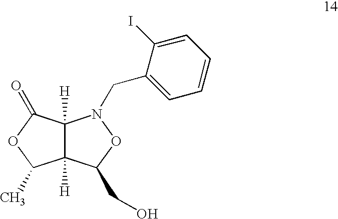

Isoxazolidine 14 was synthesized according to general method 2 using nitrone methyl ester 9 in place of nitrone methyl ester 7 and allylic alcohol 12 in place of allylic alcohol 10. Yield: 40-60%.

### Example 16

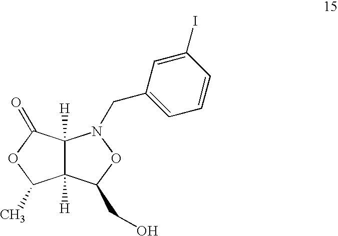

Isoxazolidine 15 was synthesized according to general method 2 using nitrone methyl ester 8 in place of nitrone methyl ester 7 and allylic alcohol 12 in place of allylic alcohol 10. Yield: 40-60%.

### Example 17

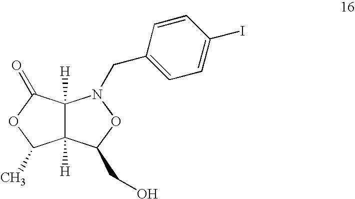

Isoxazolidine 16 was synthesized according to general method 2 using nitrone methyl ester 7 and allylic alcohol 12 in place of allylic alcohol 10. Yield: 40-60%.

### Example 18

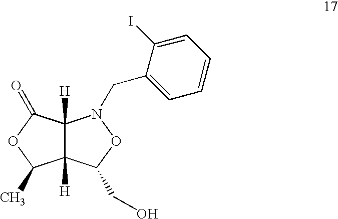

Isoxazolidine 17 was synthesized according to general method 2 using nitrone methyl ester 9 in place of nitrone methyl ester 7 and allylic alcohol 10. Yield: 40-60%.

### Example 19

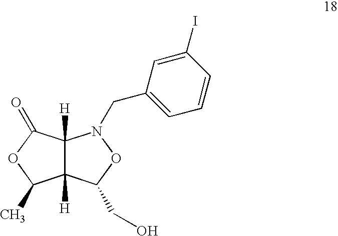

Isoxazolidine 18 was synthesized according to general method 2 using nitrone methyl ester 8 in place of nitrone methyl ester 7 allylic alcohol 10. Yield: 40-60%.

### Example 20

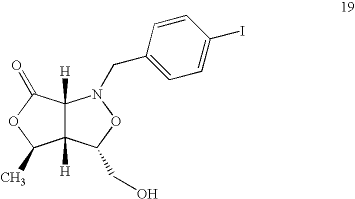

Isoxazolidine 19 was synthesized according to general method 2 using nitrone methyl ester 7 and allylic alcohol 10. Yield: 40-60%.

### Example 21

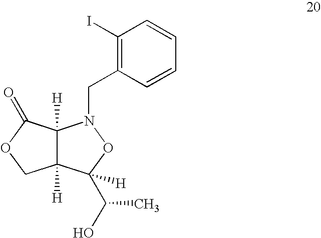

Isoxazolidine 20 was synthesized according to general method 1 using nitrone acid 6 in place of nitrone acid 4 and allylic alcohol 11. Yield: 50-60%.

### Example 22

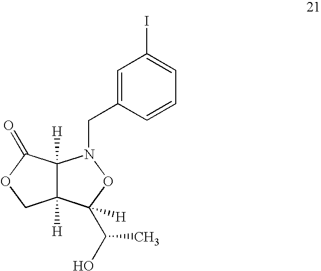

Isoxazolidine 21 was synthesized according to general method 1 using nitrone acid 5 in place of nitrone acid 4 and allylic alcohol 11. Yield: 50-60%.

### Example 23

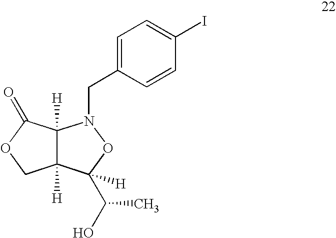

Isoxazolidine 22 was synthesized according to general method 1 using nitrone acid 4 and allylic alcohol 12. Yield: 50-60%.

### Example 24

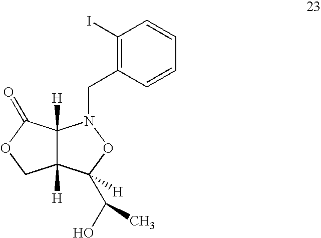

Isoxazolidine 23 was synthesized according to general method 1 using nitrone acid 6 in place of nitrone acid 4 and allylic alcohol 13 in place of allylic alcohol 11. Yield: 50-60%.

### Example 25

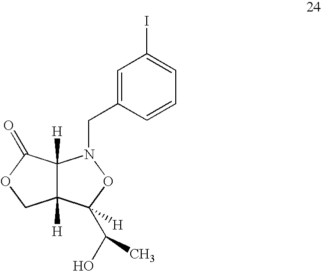

Isoxazolidine 24 was synthesized according to general method 1 using nitrone acid 5 in place of nitrone acid 4 and allylic alcohol 13 in place of allylic alcohol 11. Yield: 50-60%.

### Example 26

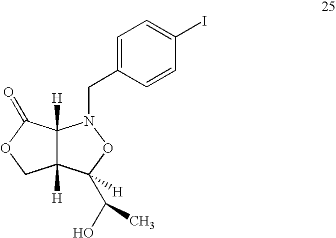

Isoxazolidine 25 was synthesized according to general method 1 using nitrone acid 4 and allylic alcohol 13 in place of allylic alcohol 11. Yield: 50-60%.

### Example 27

### Synthesis of the Allyl Silane Linker

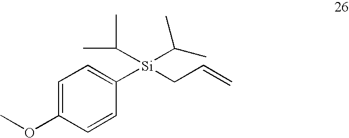

The allyl silane 26 was synthesized according to the procedure described in Schreiber et al., J. Chem. Comb. 2001,3,312-318.

### Example 28

### Modification of Mimotopes Lanterns with Silyl Linker

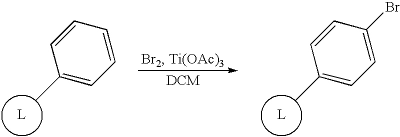

32,000 lanterns (2.22 mol of Ph rings, SynPhase-PS L-Series lanterns; Mimotopes, Clayton Victoria Australia) were added to a 22 L reactor flask containing a 5-neck detachable head and a Teflon®-screw bottom port. The head was connected to an air-driven overhead stirrer bearing a 16 cm-wide Teflon® paddle, an argon inlet, an addition funnel (250 mL), a temperature probe and an outlet for an HBr trap (1 L flask filled with 500 mL water). The reactor was flushed with argon for 15 min followed by the addition of anhydrous DCM (14.8 L). After 10 min, thallium acetate (76 g, 0.20 mol, 0.090 eq) was added. The reaction vessel was covered in aluminum foil and allowed to stir at ambient temperature for 150 min Bromine (177 g, 1.11 mol, 0.50 eq) in DCM (100 mL) was placed in the addition funnel and was added drop wise over the course of 15 min to the reactor, which warmed the reaction temperature from 19.3 to 27.0° C. Following bromine addition, the reaction was stirred for an additional 60 min. The reaction was then quenched with MeOH (1.5 L) and was allowed to stir at ambient temperature overnight. The reaction solution was drained to waste and the lanterns were washed according to the following protocol: (12 L each for 10-20 min) DCM, 3:1 THF:IPA, 3:1 THF:water, water, and THF (2×). The lanterns were stripped of solvent under reduced pressure. Bromine elemental analysis of five lanterns indicated an average bromine loading level of 34.2±0.7 μmol/lantern where 35.0 was the target (98% Br incorporation).

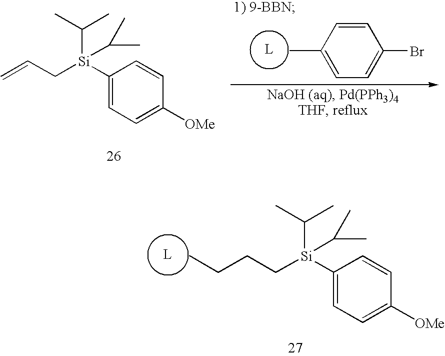

A 22 L reactor flask containing a Teflon®-screw bottom port with a detachable 5-neck head was connected to a solvent or argon inlet, an air-driven (sparkproof) overhead stirrer bearing a 16 cm-wide Teflon® paddle, a temperature probe, and two condensers. The reactor flask was placed in a 3-legged heating mantle stand and secured to the wall of a walk-in hood. Anhydrous THF (500 mL, 40 ppm H₂O by KF test) was added to the flask and the solvent was stirred vigorously to rinse the flask walls in order to remove water. The solvent was drained out to waste through the bottom port under a flow of argon. The flask was flushed with argon for 10 min and then was charged with di-isopropyl(4-methoxyphenyl)allylsilane (353 g, 1.34 mol, 1.2 eq., Maybridge # MO01086ZZ). Anhydrous THF was added (11 L). Argon was bubbled vigorously through the solution for 20 min using ⅛″-wide Teflon® hose. Then, 9-BBN (167 g, 1.34 mol., 1.2 eq.) was added and the solution was stirred at ambient temperature under argon for 2 h. An in-process-check (NMR, CDCl₃) revealed complete consumption of the allylsilane. The brominated lanterns (32,000, 1.11 mol Br, 1.0 eq.), Pd(PPh₃)₄ (65 g, 0.056 mol, 0.05 eq., Strem Chemical # 40-2150) and 2 N NaOH (1.34 L, 2.69 mol, 2.4 eq.) were added under a stream of argon. The reaction mixture was heated to an internal temperature of 65° C. under a positive flow of argon with stirring for 40 h. The reaction was cooled, drained and washed with the following solvents in this order (10 L, 10-20 min each): THF, 3:1 THF:IPA, 3:1 THF:1 N NaCN (aqueous) (1 h or until all black color on lanterns is gone), water (2×), 3:1 THF:water, THF (2×) and DCM. The lanterns were stripped of solvent under reduced pressure. Silicon elemental analysis of five lanterns indicated an average silicon loading level of 22.2±2.2 μmol/lantern. Bromine analysis indicated 5.5 μmol/lantern of residual bromine.

### Example 29

Loading and Modification of the Isoxazolidine Cores

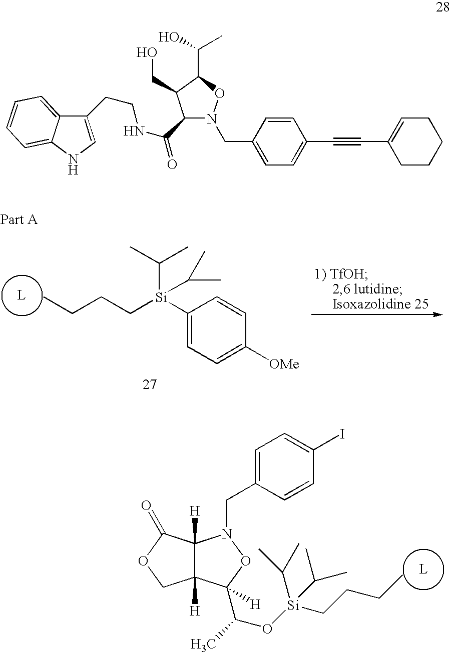

1808 lanterns were placed in a flame dried 2 L flask. A stir bar was added and the flask was purged with nitrogen and capped with a rubber septa. 1.2 L of anhydrous DCM was added to the flask and the lanterns were allowed to sit in this solution for 10 minutes and then the solvent was removed. A 3% triflic acid solution in anhydrous DCM (1.2 L, 393 mmol, 3%, v/v) was added and the lanterns were stirred gently for 20 minutes. The triflic acid solution was then removed via cannula. 1.2 L of anhydrous DCM and 2,6-lutidine was added (62 mL, 532 mmol). The lanterns were stirred in this solution for 10 minutes. Dry Isoxazolidine 25 (10 g, 38 mmol) was then added. The resulting mixture was stirred for 18 h. At which point the reaction solution was decanted and the lanterns were washed according to the following protocol: (2×10 minutes) DCM (1.5 L), THF (1.5 L), THF:IPA (3:1, 1.5 L), THF: Water (3:1, 1.5 L), THF:IPA (3:1, 1.5 L), and THF (1.5 L). The lanterns were then dried under reduced pressure. Loading level determination: 5 lanterns were each placed into 5 mL polypropylene containers. To each container was added 300 μL of THF and 50 μL of HF-pyridine. The lanterns were allowed to sit in this solution for 6 h. At which point 500 μL was added and the lanterns were allowed to sit in this solution for an additional 15 minutes. The reaction solution was then transferred to a tared flask and concentrated under reduced pressure to afford Isoxazolidine 25. This material was massed and the average loading level was calculated to 14 μmol/lantern.

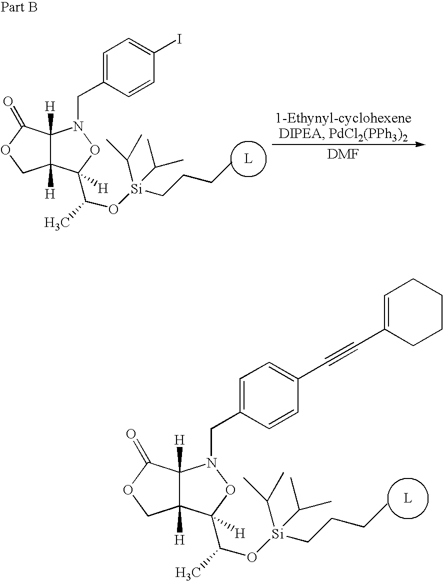

376 lanterns with isoxazolidine core material loaded on them were placed in a 500 mL flask. The flask was flushed with nitrogen and capped. Palladium bistriphenylphosphine (7.92 g, 11.28 mmol) and copper iodide (3.22 g, 16.90 mmol) were added to the flask. The reaction vessel was flushed with nitrogen again, capped, and anhydrous DMF (300 mL) was added. Diisopropylethylamine (30 mL, 172.23 mmol,) was then added, followed by the addition of 1-ethynyl-cyclohexene (110 mmol) via syringe. The reaction vessel was then shaken gently for 2 h. The reaction solution was then decanted and the lanterns were washed according to the following protocol: (2×10 minutes) DMF (300 mL), THF (300 mL), THF:IPA (3:1, 300 mL), THF: Water (3:1, 300 mL), THF:IPA (3:1, 300 mL), THF (300 mL), DCM (300 mL). Reaction conversion determination: 1 lantern was placed into a 5 mL polypropylene container. 300 mL of THF and 50 μL of HF-pyridine were added to the container. The lantern was allowed to sit in this solution for 6 h. At which point 500 μL was added and the lantern was allowed to sit in this solution for an additional 15 minutes. The reaction solution was then transferred and concentrated under reduced pressure to afford the Sonogashira product.

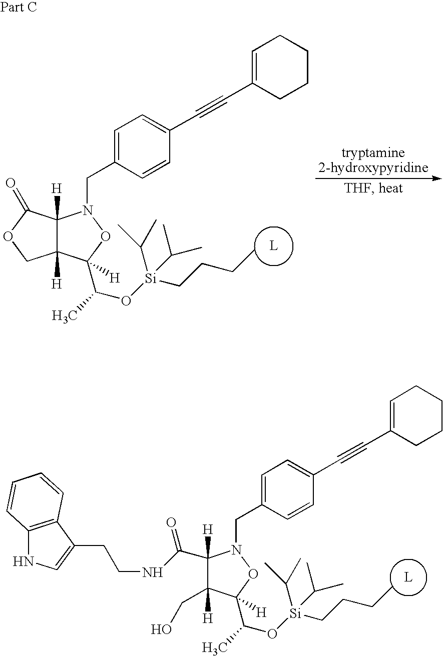

374 lanterns were placed into a 500 mL round bottom flask. 0.35 M 2-hydroxypyridine in anhydrous THF was added (300 mL, 105 mmol). The flask was flushed with nitrogen and capped with a rubber septa. Tryptamine was then added (279 mmol) and the reaction was heated to 50° C. for 16 h. The solvent was then removed and the lanterns were washed according to the following protocol: (2×10 minutes) THF (300 mL), THF:IPA (3:1, 300 mL), THF: Water (3:1, 300 mL), THF:IPA (3:1, 300 mL), THF (300 mL), DCM (300 mL). Reaction conversion determination: 1 lantern was placed into a 5 mL polypropylene container. 160 μL of THF, 200 μL of pyridine and 40 μL of HF-pyridine were added to the contained. The lantern was allowed to sit in this solution for 1 h. At which point 500 μL was added and the lantern was allowed to sit in this solution for an additional 15 minutes. The reaction solution was then transferred and concentrated under reduced pressure to afford the product.

Isoxazolidine 28 was characterized by LC-MS analysis. The general procedures and conditions used for analytical analysis used in this and other examples are presented below.

Conditions for LC-MS Analysis

| | |
|---|---|
| Mass Spectrometer: | Waters ZQ |
| HPLC: | Waters 2795 Alliance HT |
| Diode Array: | Waters 2696 |

Mass Spectrometer Conditions:

Mass spectrometer ionization mode: electro-spray
with positive negative switching.

| | |
|---|---|
| Mass Range | 150-1000 Daltons |
| Capillary (KV) | 3.2 |
| Cone (V) | 35 |
| Extractor (V) | 3 |
| RF Lens | 0 |
| Source Temperature | 120° C. |
| Desolvation Temp. | 350° C. |
| Cone Gas | 25 L/H |
| Desolvation Gas | 550 L/H |

HPLC Conditions:

| Mobile phases: | A: Water 95% Acetonitrile 5% Formic Acid 0.1% | | | |
|---|---|---|---|---|
| | B: Water 5% Acetonitrile 95% Formic Acid 0.1% | | | |
| Flow rate: | 1.00 mL/minute | | | |
| Column: | Waters Symmetry 4.6 mm by 50 mm 5 micron C18 | | | |
| Column | 50° C. | | | |
| Temperature | | | | |
| Gradient: | A | B | Time | Flow |
| | 85 | 15 | 0.0 | 1.0 |
| | 85 | 15 | 1.0 | 1.0 |
| | 0 | 100 | 5.0 | 1.0 |
| | 0 | 100 | 6.0 | 1.0 |
| | 85 | 15 | 6.1 | 1.5 |
| | 85 | 15 | 7.0 | 1.5 |
| | 85 | 15 | 8.0 | 1.0 |

| | |
|---|---|
| Injection volume: | 5 μL |
| Diode Array | Wavelength array: 220 nm-400 nm |
| conditions: | Resolution: 1.2 nm |

Sample concentrations are normally run at 0.2 mg/mL unless otherwise stated.

Conditions for MS-TOF Analysis

| | |
|---|---|
| Mass Spectrometer: | Micromass LCT |
| HPLC: | Waters 2795 Alliance HT |
| Diode Array: | Waters 2696 |

Mass Spectrometer Conditions:

Mass spectrometer ionization mode: electro-spray positive

| | |
|---|---|
| Mass Range | 150-1000 Daltons |
| Capillary (KV) | 3.2 |
| Cone (V) | 35 |
| Extractor (V) | 3 |
| RF Lens | 0 |
| Source Temperature | 120° C. |
| Desolvation Temp. | 350° C. |
| Cone Gas | 25 L/H |
| Desolvation Gas | 450 L/H |

HPLC Conditions:

| Mobile | A: Water with Formic Acid 0.1% | | | |
|---|---|---|---|---|
| phases: | B: 65% Methanol/35% 2-Propanol with Formic Acid 0.1% | | | |
| Flow rate: | 1.00 mL/minute | | | |
| Column: | Varian Polaris 2.1 mm by 50 mm 5 micron C18 | | | |
| Column | 50° C. | | | |
| Temperature | | | | |
| Gradient: | A | B | Time | Flow |
| | 90 | 10 | 0.0 | 1.0 |
| | 90 | 10 | 0.5 | 1.0 |
| | 10 | 90 | 3.2 | 1.0 |
| | 10 | 90 | 3.4 | 1.0 |
| | 0 | 100 | 3.5 | 1.0 |
| | 0 | 100 | 4.0 | 1.0 |

Injector system runs in two column regeneration mode so that column equilibration occurs during the rime of the next sample analyzed.

| | |
|---|---|
| Injection volume: | 5 μL |
| Diode Array conditions: | Wavelength array: 220 nm-400 nm |
| | Resolution: 1.2 nm |

Sample concentrations are normally run at 2.0 mg/mL unless otherwise stated.

### Example 30

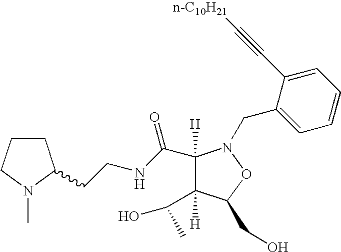 Compound 29 was synthesized according to the procedure described in Example 29 using isoxazoline 14 in place of isoxazolidine 25, dodec-1-yne in place of 1-ethynyl-cyclohexene and 2-(1-methyl-pyrrolidin-2-yl)-ethylamine in place of tryptamine. MS (ESI(+)) m/e 556.35

### Example 31

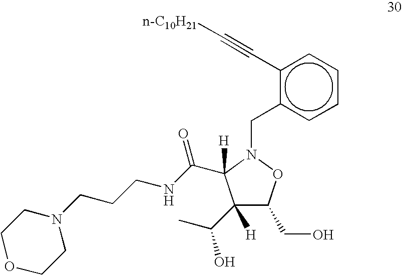

Compound 30 was synthesized according to the procedure described in Example 29 using isoxazolidine 17 in place of isoxazolidine 25, dodec-1-yne in place of 1-ethynyl-cyclohexene, and 3-morpholin-4-yl-propylamine in place of tryptamine.

### Example 32

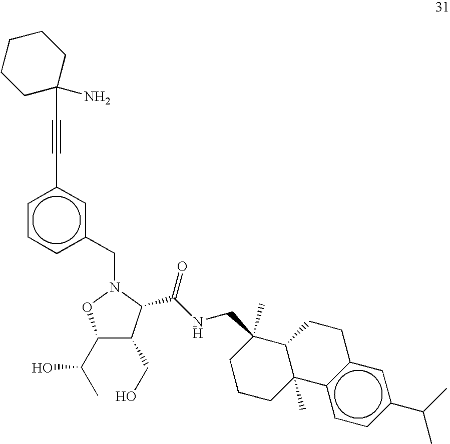

Compound 31 was synthesized according to the procedure described in Example 29 using isoxazolidine 21 in place of isoxazolidine 25, 1-ethynyl-cyclohexylamine in place of 1-ethynyl-cyclohexene, and (+)-dehydroabietylamine in place of tryptamine.

### Example 33

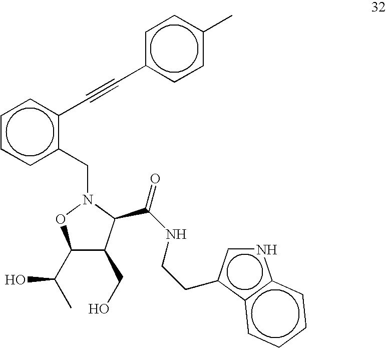

Compound 32 was synthesized according to the procedure described in example 29 using isoxazolidine 19 in place of isoxazolidine 25, 1-ethynyl-4-methyl-benzene in place of 1-ethynyl-cyclohexene, and tryptamine. MS (ESI(+)) m/e 537.96 (M+H)⁺.

### Example 34

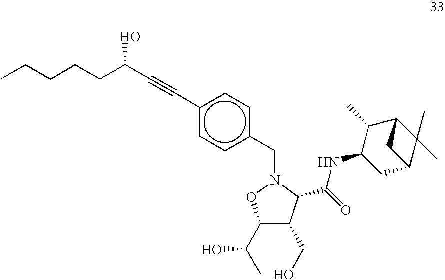

Compound 33 was synthesized according to the procedure described in Example 29 using isoxazolidine 21 in place of isoxazolidine 25, (R)-oct-1-yn-3-ol in place of 1-ethynyl-cyclohexene, and (−)-isopinocampheylamine in place of tryptamine. MS (ESI(+)) m/e 541.39 (M+H)⁺.

### Example 35

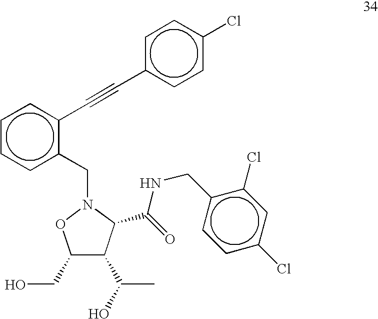

Compound 34 was synthesized according to the procedure described in example 29 using isoxazolidine 14 in place of isoxazolidine 25, 1-chloro-4-ethynyl-benzene in place of 1-ethynyl-cyclohexene, and 2,4-dichloro-benzylaminein place of tryptamine. MS (ESI(+)) m/e 573.03 (M+H)⁺.

### Example 36

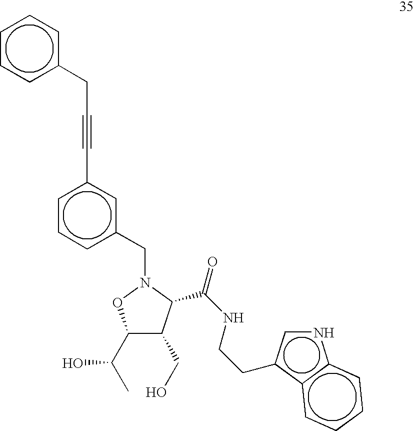

Compound 35 was synthesized according to the procedure described in Example 29 using isoxazolidine 21 in place of isoxazolidine 25, prop-2-ynyl-benzene in place of 1-ethynyl-cyclohexene, and tryptamine.

### Example 37

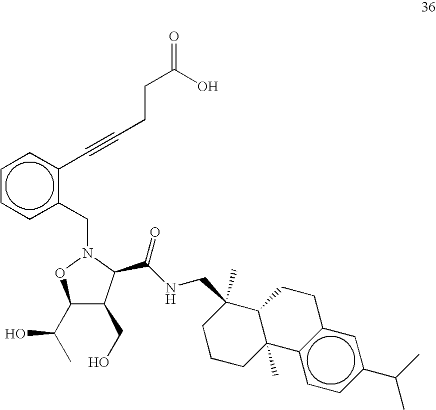

Compound 36 was synthesized according to the procedure described in Example 29 using isoxazolidine 17 in place of isoxazolidine 25, pent-4-ynoic acid in place of 1-ethynyl-cyclohexene, and (+)-dehydroabietylamine in place of tryptamine.

### Example 38

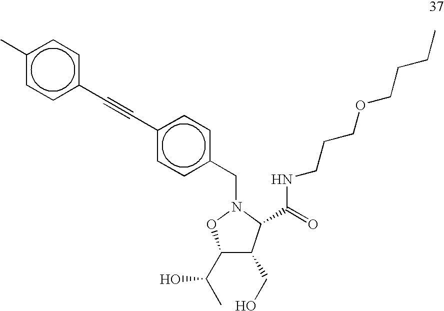

Compound 37 was synthesized according to the procedure described in Example 29 using isoxazolidine 21 in place of isoxazolidine 25, 1-ethynyl-4-methyl-benzene in place of 1-ethynyl-cyclohexene, and 3-butoxy-propylamine in place of tryptamine. MS (ESI(+)) m/e 509.22 (M+H)⁺.

### Example 39

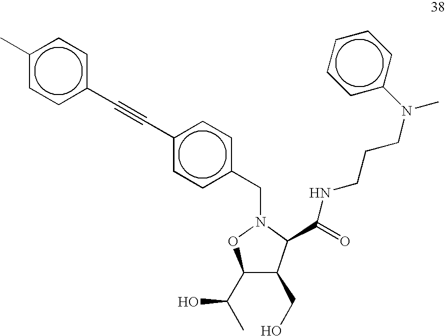

Compound 38 was synthesized according to the procedure described in Example 29 using isoxazolidine 25 in place of isoxazolidine 25, 1-ethynyl-4-methyl-benzene in place of 1-ethynyl-cyclohexene, and N-methyl-N-phenyl-propane-1,3-diamine in place of tryptamine. MS (ESI(+)) m/e 542.25 (M+H)⁺.

### Example 40

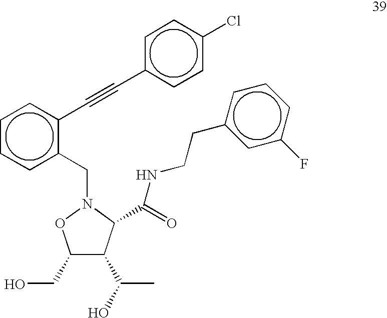

Compound 39 was synthesized according to the procedure described in Example 29 using isoxazolidine 14 in place of isoxazolidine 25, 1-ethynyl-4-chloro-benzene in place of 1-ethynyl-cyclohexene, and 2-(3-fluoro-phenyl)-ethylamine in place of tryptamine.

### Example 41

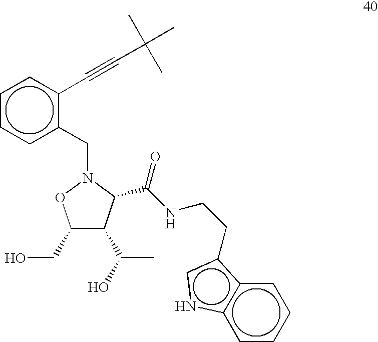

Compound 40 was synthesized according to the procedure described in Example 29 using isoxazolidine 14 in place of isoxazolidine 25, t-butyl-acetylene in place of 1-ethynyl-cyclohexene, and tryptamine.

### Example 42

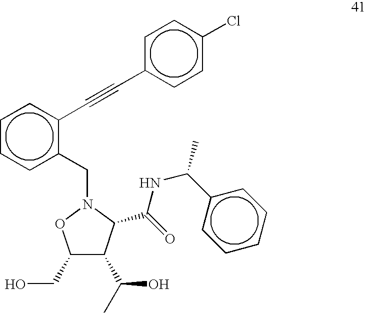

Compound 41 was synthesized according to the procedure described in Example 29 using isoxazolidine 14 in place of isoxazolidine 25, 1-ethynyl-4-chloro-benzene in place of 1-ethynyl-cyclohexene, and (R)-1-phenyl-ethylamine in place of tryptamine. MS (ESI(+)) m/e 518.87 (M+H)⁺.

### Example 43

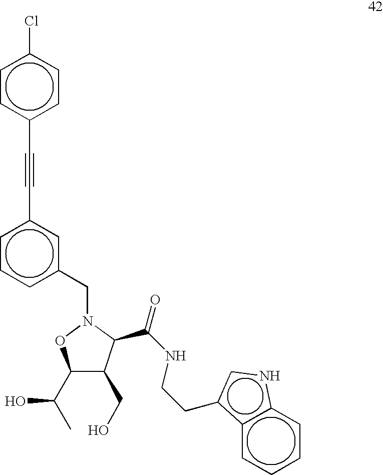

Compound 42 was synthesized according to the procedure described in Example 29 using isoxazolidine 24 in place of isoxazolidine 25, 1-ethynyl-4-chloro-benzene in place of 1-ethynyl-cyclohexene, and tryptamine. MS (ESI(+)) m/e 557.66 (M+H)⁺.

### Example 44

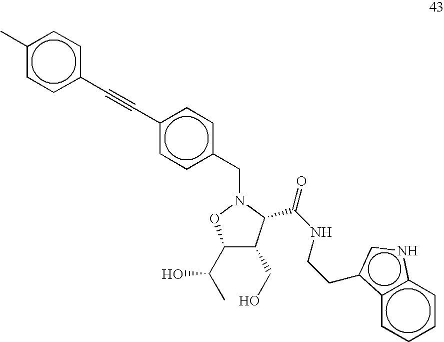

Compound 43 was synthesized according to the procedure described in Example 29 using isoxazolidine 22 in place of isoxazolidine 25, 1-ethynyl-4-methyl-benzene in place of 1-ethynyl-cyclohexene, and tryptamine. MS (ESI(+)) m/e 537.98 (M+H)⁺.

### Example 45

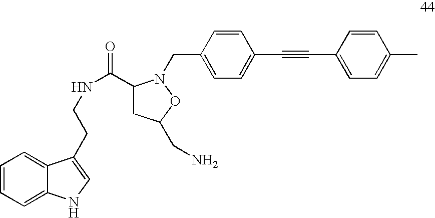

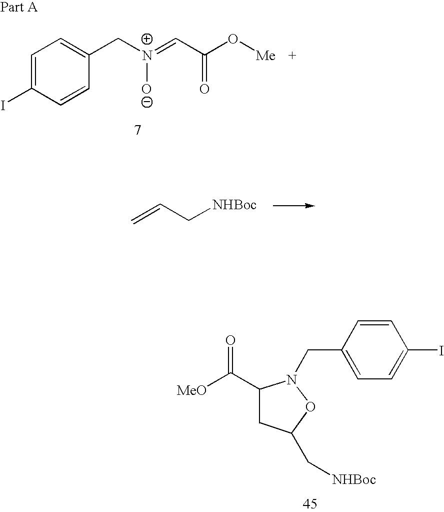

Para-iodo nitrone carboxylic acid methyl ester 7 (0.46 g) was added to a toluene (3 mL) solution of tert-butyl N-allylcarbamate (0.23 g) in a dry 25 mL round bottom flask. The flask was then heated up at 90° C. for 16 h under a water condenser. The reaction mixture was cooled to rt, concentrated to give 0.7 g of crude product. Purification with flash chromatography on silica gel, (1:2 ethyl acetate EtOAc/hexanes to 1:1) gave desired product 540 mg.

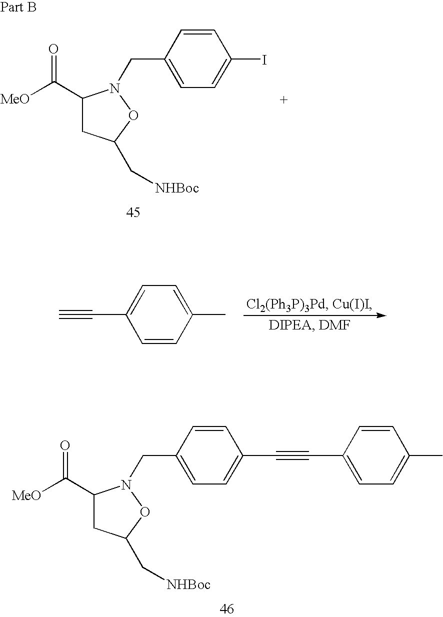

To a solution of isoxazolidine 45 (0.24 g) in 4 mL of anhydrous DMF in a round bottom flask under nitrogen was added dichlorobis(triphenylphosphine)palladium(II) catalyst (0.11 g, 0.3 equiv.), copper iodide (0.04 g, 0.4 equiv.), and 1-ethynyl-4-methyl-benzene (0.12 g, 2 equiv.). Hunig's base (0.26 mL, 3 equiv.) was added slowly via syringe and the solution immediately turned dark brown. The reaction mixture was stirred at rt for 3 h and was quenched by adding water and diluted with ethyl acetate 50 mL). The organic layer was washed with water (5×10 mL), brine and dried over MgSO4. Filtration and concentration in vacuo gave a black tar, which was purified on silica gel column (3:1 hexanes/ethyl acetate to 1:1 hexanes/ethyl acetate) gave desired pure product 130 mg.

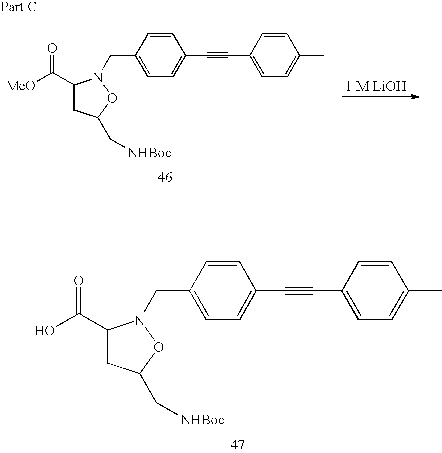

To a solution of 46 (30 mg) in 2 mL MeOH was added 1 M LiOH solution 0.25 mL at rt. The reaction mixture was then stirred at rt for 1 h. Ethyl acetate (50 mL) was added and the resulting mixture was washed with brine, dried and concentrated to give crude product 36 mg.

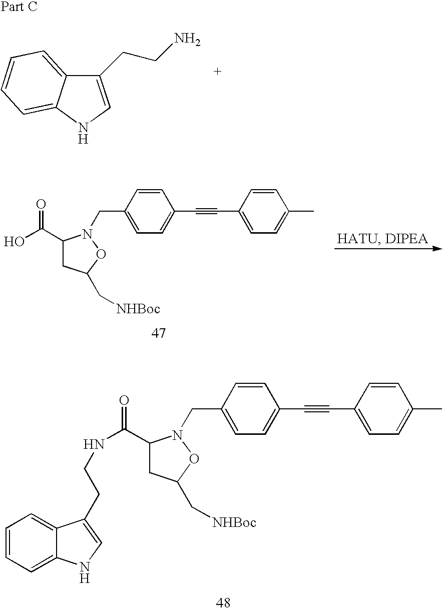

The isoxazolidine 47 (36 mg), tryptamine (15 mg, 1.2 equiv.), and HATU (36 mg, 1.2 equiv.) were dissolved into 1.5 mL of anhydrous dichloromethane and 0.5 mL of N,N-dimethylformamide under nitrogen. Hunig's base (0.03 mL, 2 equiv.) was added slowly via syringe. The mixture was stirred at rt for 12 h. Ethyl acetate (50 mL) was used to dilute the reaction mixture. The resulting mixture was washed with water (3×20 mL) and brine. The organic solution was then dried and concentrated to give 45 mg crude product, which was purified on silica gel column (3:1 hexanes/ethyl acetate to 1:1 hexanes/ethyl acetate) to give 22 mg of pure desired product.

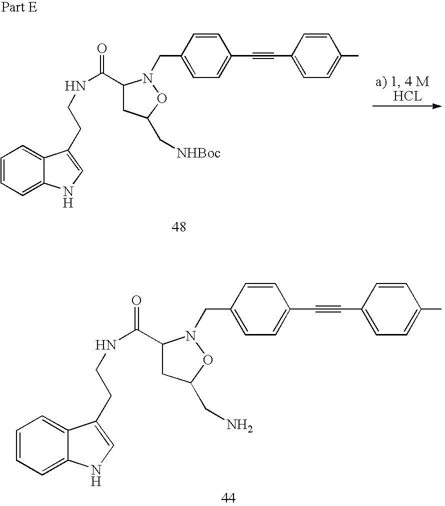

To a solution of 48 (20 mg) in 0.5 mL EtOAc was added 4 M HCl/dioxane solution 0.08 mL at rt. The reaction mixture was then stirred at rt for 1 h, and then another 0.3 mL HCl solution was added. After another 3 h, the reaction mixture was concentrated. The crude product was then washed with ethyl acetate and filtered. Half of the crude product (5 mg) was dissolved into 0.2 mL MeOH and treated with 1 mL of sat. NaHCO₃. EtOAc (30 mL) was used to extract the resulting mixture. The organic layer was washed with brine, dried and concentrated to give 4 mg of the desired product. MS (ESI(+)) m/e 492.9 (M+H)⁺.

### Example 46

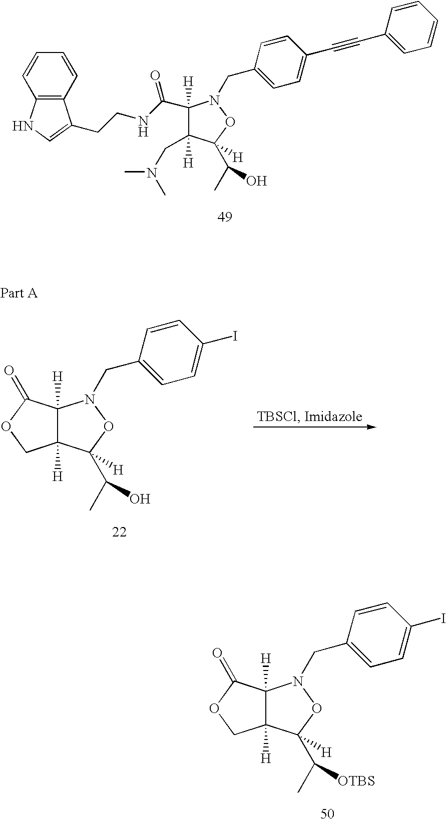

Compound 22 (0.5 g) and tert-butyldimethylsilyl chloride (0.2 g, 1.2 equiv.) were dissolved in DMF (10 mL) and cooled to 0° C. Imidazole (0.1 g, 1.5 equiv.) was then added. The reaction mixture was stirred at rt for overnight. Work up was done by adding 10 mL water, and then extracts the aqueous layer with ethyl acetate (4×30 mL). The organic layers were washed with water (2×), brine, and dried to give desired crude product 0.54 g.

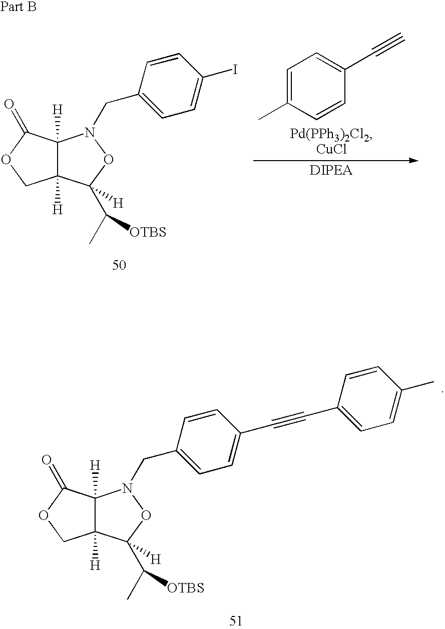

To a solution of 50 (0.54 g) in 4 mL anhydrous DMF in a round bottom flask under nitrogen was added Dichlorobis(triphenylphosphine)palladium(II) catalyst (0.23 g, 0.3 equiv.), copper iodide (0.08 g, 0.4 equiv.), and 1-ethynyl-4-methyl-benzene (0.24 g, 2 equiv.). Hunig's base (0.52 mL, 3 equiv.) was added slowly via syringe and the solution immediately turned dark brown. The reaction mixture was stirred at rt for 3 h and was then quenched by adding water and diluted with ethyl acetate 50 mL). The organic layer was washed with water (5×10 mL), brine and dried over MgSO₄. Filtration and concentration in vacuo gave 0.62 g black tar.

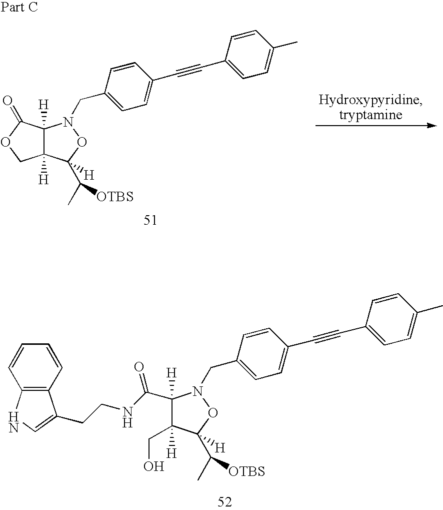

Compound 51 (0.62 g), hydroxypyridine (0.5 g, 4 equiv.), and tryptamine (1.0 g, 5 equiv.) were dissolved in 6 mL of anhydrous THF. The reaction mixture was heated up to 50° C. for 16 h. Solvent was then removed under reduced pressure and the crude was redissolved in EtOAc. The resulting solution was washed with water, brine, dried over MgSO₄, and concentrated to give crude product 0.97 g. Flash chromatography on silica gel (EtOAc/Hex=1:2 to 1:1 then 2:1) afforded 0.75 g of the desired product.

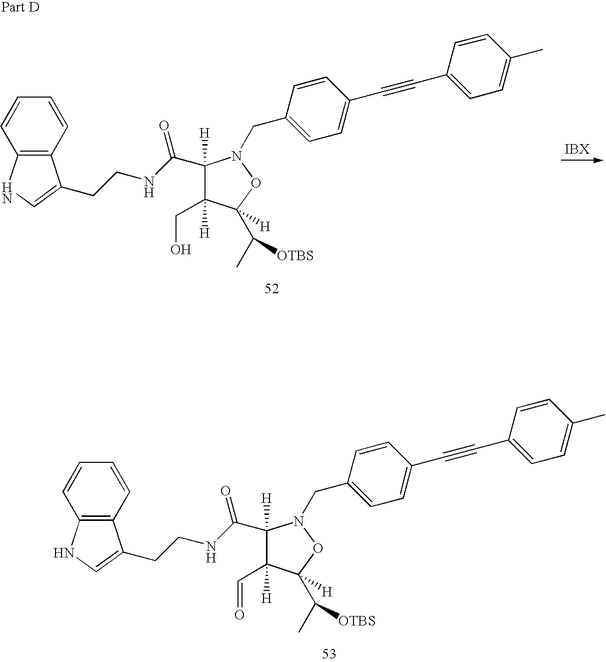

Compound 52 (0.13 g) was dissolved in 1.5 mL DMSO. IBX (0.11 g, 2 equiv.) was then added. The reaction was run for 3.5 h. Ethyl acetate (100 mL) was added to the reaction mixture and resulted solution was washed with water (4×15 mL), brine, and dried, filtered, and concentrated to afford 0.14 g of the crude product.

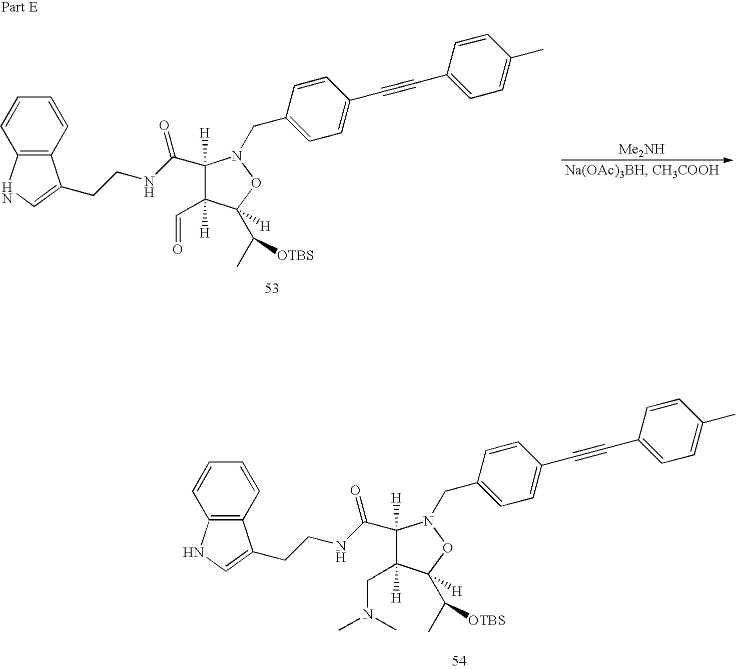

Compound 53 (0.14 g), dimethylamine (0.16 mL, 2 M in THF, 1.5 equiv.), sodium triacetoxyborohydride (70 mg, 1.5 equiv.) and acetic acid (0.01 mL) were dissolved in 2 mL of dry THF under nitrogen. After 18 h the reaction was quenched with saturated ammonium chloride. This mixture was extracted with DCM (3×5 mL). The organic layers were collected, washed with brine, dried over MgSO₄, and concentrated under reduced pressure to afford 150 mg crude product. The crude was purified by flash chromatography on silica gel (1:1 hexanes/ethyl acetate to 100% ethyl acetate) to afford 50 mg of the desired product.

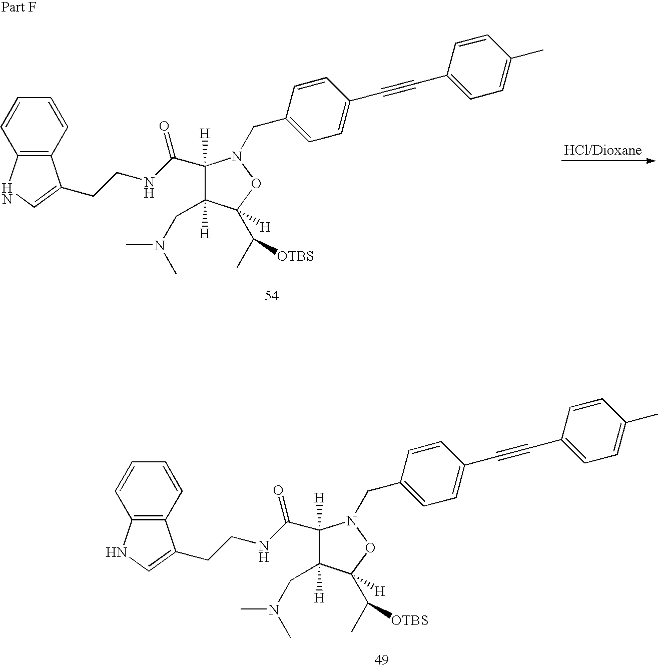

Compound 54 was dissolved in 1.5 mL of EtOAc, and then 0.5 mL of 4 M HCl in dioxane was added slowly. The mixture was concentrated after 1 h and treated with 2 mL of sat. NaHCO₃. Ethyl acetate (40 mL) was used to extract the product, which was dried (Na₂SO₄) and concentrated. Flash chromatography on silica gel (100% EtOAc to 5:95 EtOAc: MeOH, to 10:90 EtOAc: MeOH) to afford 17 mg of desired product 49. MS (ESI(+)) m/e 565.3 (M+H)⁺.

### Example 47

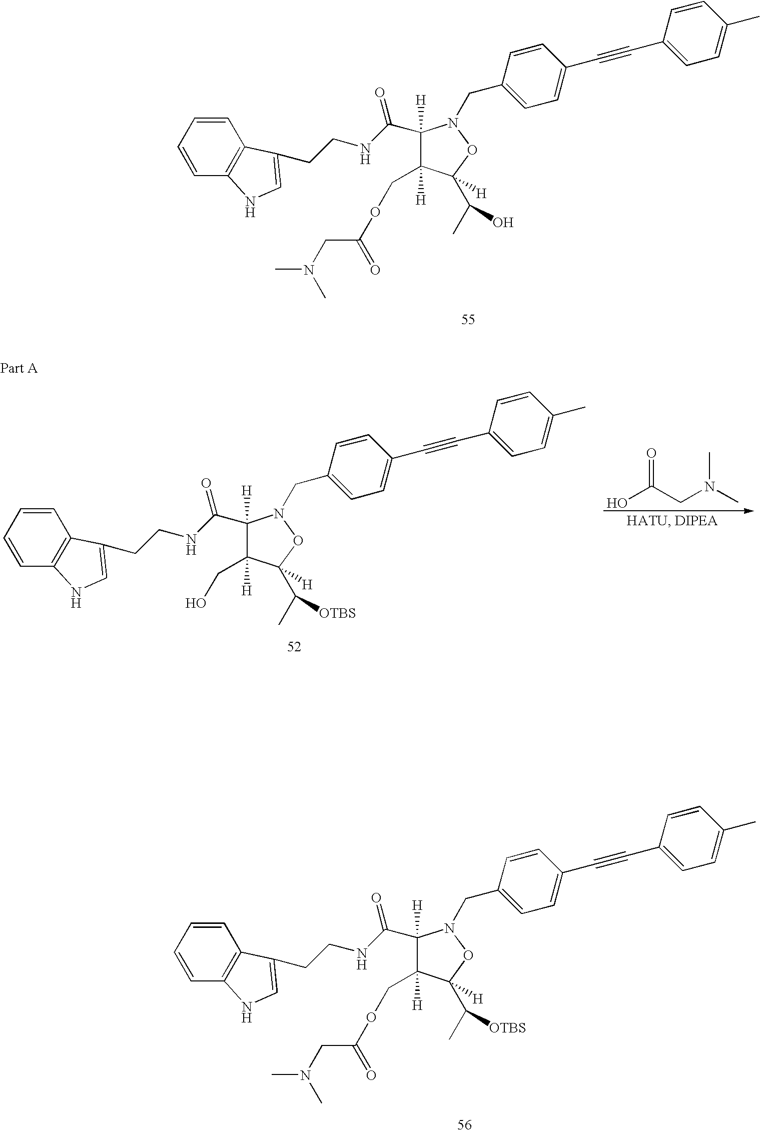

Compound 52 (0.300 g, 0.46 mmol, 1 equiv), dimethylglycine (0.095 g, 0.92 mmol, 2 equiv), and HATU (0.35 g, 0.92 mmol, 2 equiv) were dissolved in anhydrous dichloromethane (8 mL) under nitrogen. Diisopropylethyl amine (0.18 g, 0.24 mL, 1.4 mmol, 3 equiv) was then added drop wise. The mixture was stirred at rt for 18 h and was then diluted with 100 mL dichloromethane. The resulting solution was washed with water, brine, dried over Na₂SO₄ and concentrated to give 500 mg crude product. Flash chromatography on silica gel (Hexanes/ethyl acetate=1:1 to 1:6 to 10% MeOH in EtOAc) afforded 0.27 g of desired product 56.

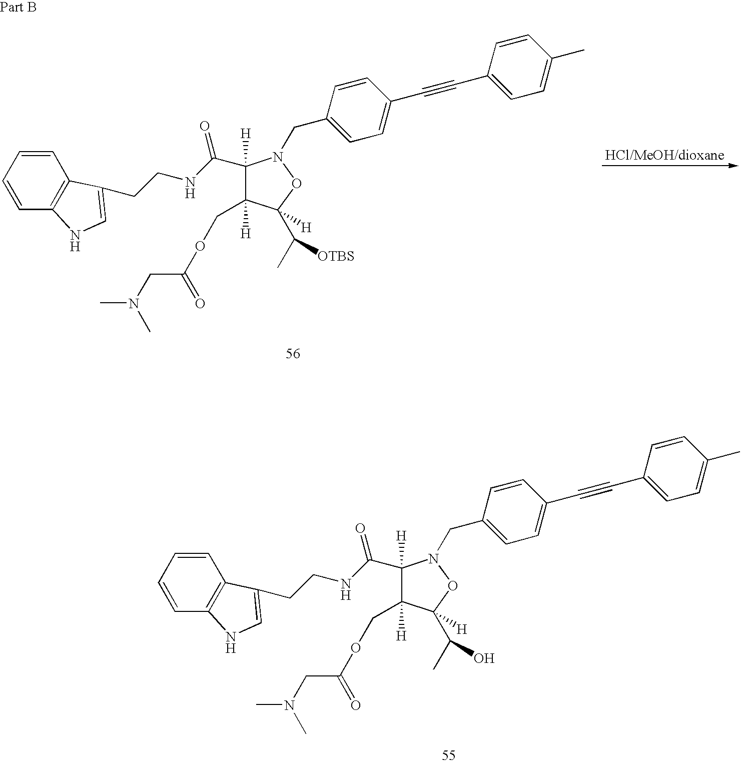

Compound 56 (0.2 g) was dissolved in 4 mL of MeOH under nitrogen. 1.4 mL of 4 M HCl in dioxane was then added at rt. After 2 h, the reaction mixture was concentrated to dryness. EtOAc (10 mL) and sat. NaHCO₃ (5 mL) were added to the residue. This mixture was diluted with EtOAc (60 mL). The organic layer was separated and washed with brine, dried over Na₂SO₄, and concentrated to give crude product 230 mg. Flash chromatography on silica gel (EtOAc, then 10% MeOH in EtOAc) afford 0.140 g of desired product 55.

### Example 48

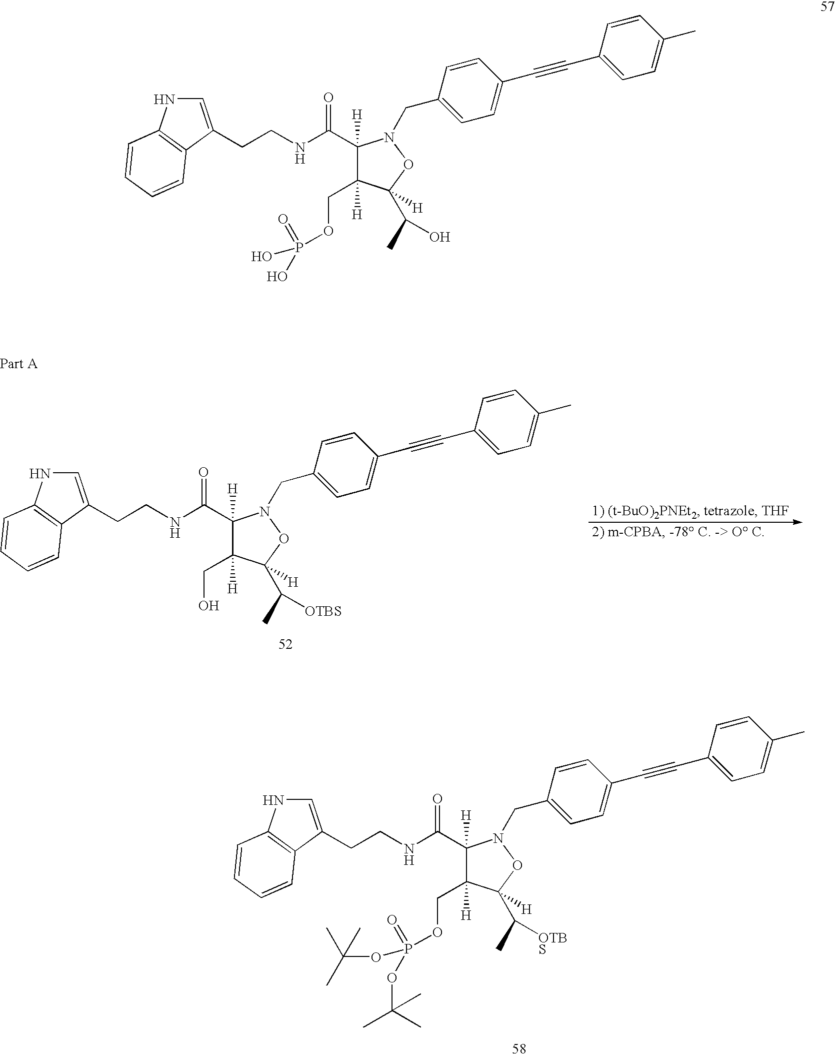

To a solution compound 52 (0.94 g) in 4 mL of anhydrous THF under nitrogen was added 1.5 equiv. of di-tert-butylphosphoramidite via syringe followed by tetrazole (0.45 M solution in acetonitrile). The reaction mixture was stirred at room temperature. After 1.5 h another 1.5 equiv. of di-tert-butylphosphoramide was added. After another 2 h, the reaction mixture was cooled to −78° C., 1.5 equiv. of m-CPBA was added and 20 min later, the cooling bath was warmed up to 0° C. After 10 minutes the reaction solution was concentrated under reduced pressure. The resulting residue was purified by column chromatography (1:1 hexanes/EtOAc) to yield the desired product in 95% yield.

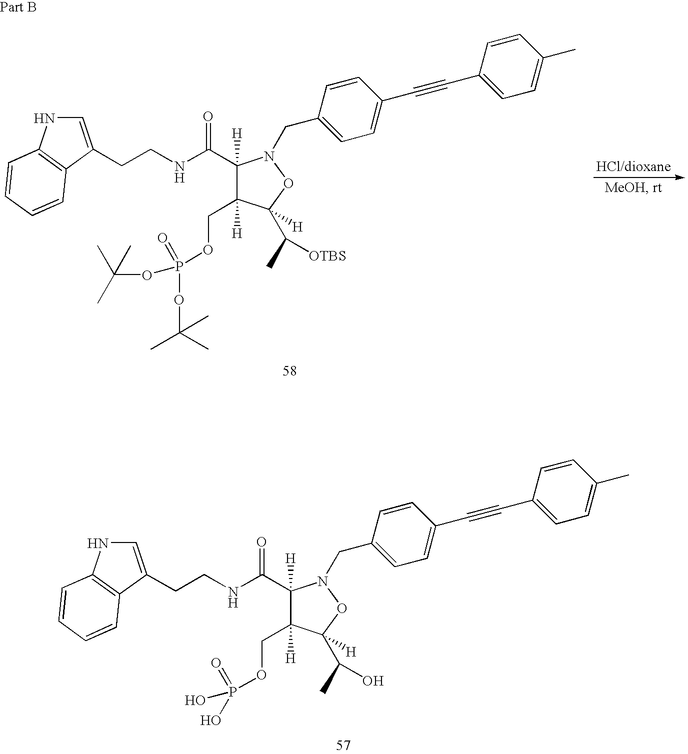

Compound 58 was dissolved in 15 mL anhydrous MeOH under nitrogen and 1.8 mL of 4 M HCl in dioxane was added and the reaction mixture was stirred at room temperature for 5 h. Another 0.4 mL of 4 M HCl was added and the mixture was concentrated and saturated Na₂CO₃ (15 mL) was added. The aqueous layer was extracted twice with EtOAc (2×15 mL). These extracts were discarded. The aqueous layer was then acidified by the addition of dilute HCl until the pH of the solution was 2. Then EtOAc (3×40 mL) was used to extract the aqueous layer. All of the organic fractions were combined, dried over Na₂SO₄, and concentrated to afford the 0.200 g of the desired product 57.

### Example 49

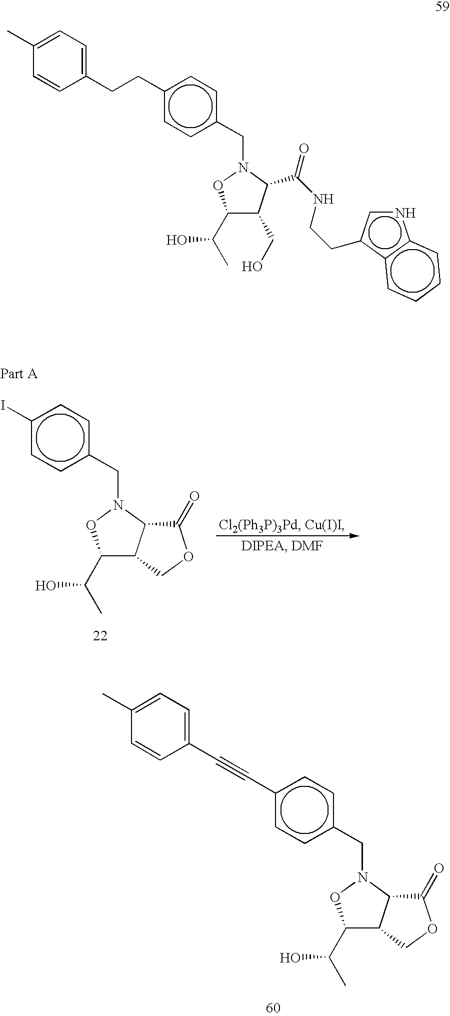

Compound 60 was synthesized according to the procedure described in the synthesis of compound 51.

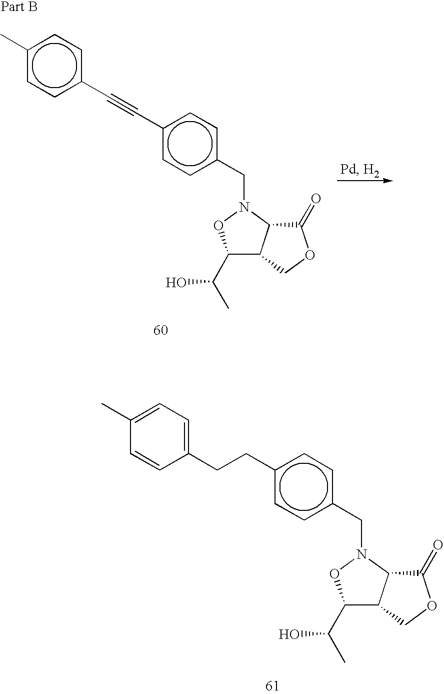

Compound 60 was methanol (0.05 M). Add palladium on carbon (1 eq. by weight). Stirred under a balloon atmosphere of hydrogen (1 atm). TLC showed completion of reaction after 5 h. Allowed to stir for another 2 h after which solution was concentrated in vacuo. Dilute in ethyl acetate and flashed through a short silica gel plug to removed carbon. 98% yield. This material was carried forward to lactone opening step. 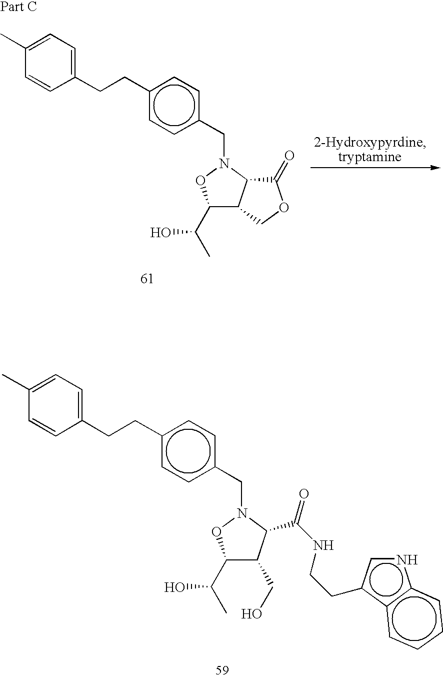
Compound 59 was synthesized according to the procedure described in the synthesis of compound 52 started to from compound 61. Yield 85%. MS (ESI(+)) m/e 542.34 (M+H)⁺.

### Example 50

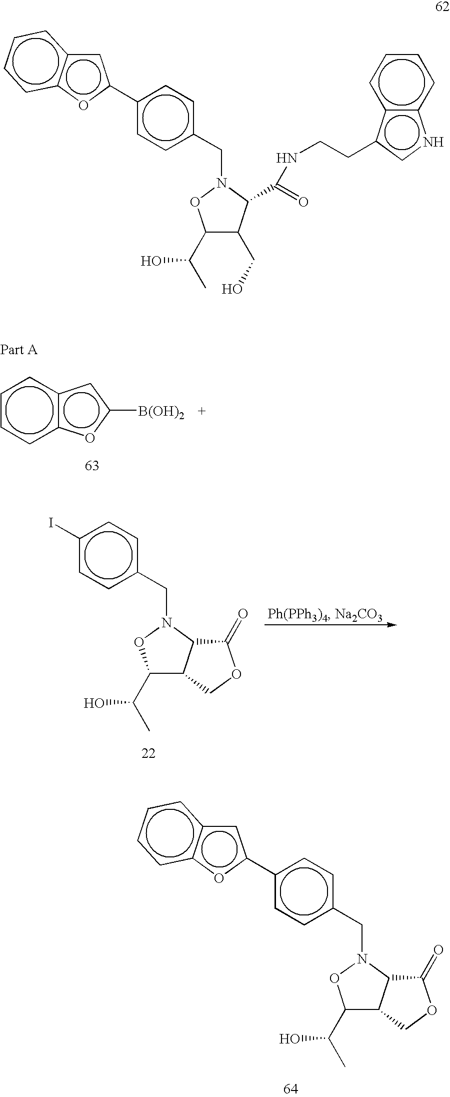

Compound 22 (97 mg, 0.257 mmol, 1.0 eq.) was dissolved in anhydrous THF (10 mL) and put under nitrogen. Boronic acid 63 (1.03 mM, 4 eq.) and sodium carbonate (1.03 mmol, 4 eq.) were added to the reaction vessel. Distilled water (1 mL) was then added, followed by addition of tetrakis(triphenylphosphine)palladium catalyst (0.0257 mmol, 0.1 eq.). Reaction vessel was then equipped with a reflux condenser and stirred at 70° C. for 2 h. The reaction mixture was then cooled to rt and partitioned between DCM/distilled water. The organic layer was collected, washed with brine, dried over MgSO₄, filtered and concentrated under reduced pressure. The crude was purified by flash chromatography to yield 29 mg of the desired product.

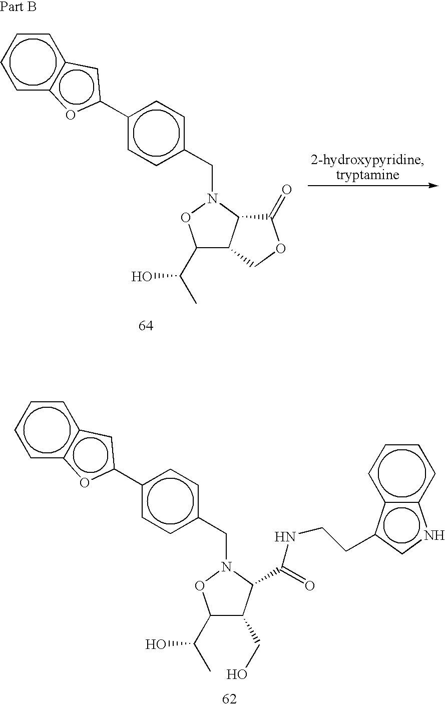

Compound 62 was synthesized according to the procedure described in the synthesis of 52 starting from compound 64. Yield 85%. MS (ESI(+)) m/e 540.24 (M+H)⁺.

### Example 51

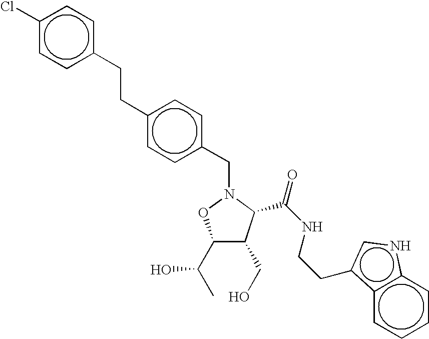

Compound 65 was synthesized according to the procedure described in the synthesis of 59, but 1-ethynyl-4-chloro-benzene was used in place of 1-ethynyl-4-methyl-benzene. MS (ESI(+)) m/e 562.0 (M+H)⁺.

### Example 52

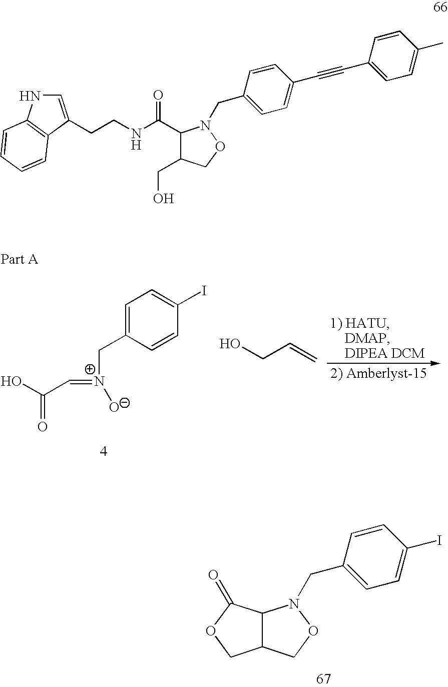

To a solution of allyl alcohol (1.10 eq., 1.08 mmol, 62.8 mg) in DCM (3 mL) was added nitrone carboxylic acid 4 (1.00 eq., 0.983 mmol, 300 mg) followed by HATU (2.00 eq., 1.97 mmol, 748 mg) and DMAP (1.50 eq., 1.48 mmol, 180 mg). The reaction mixture was cooled to 0° C. and DIPEA (1.50 eq., 1.48 mmol, 191 mg) was added drop wise. The reaction mixture was stirred at this temperature for 2 h and then allowed to warm to r.t. over another h. The reaction mixture was quenched with water and diluted with ethyl acetate. The organic layer was washed with water, 5% sodium bicarbonate, and then with brine. The organic extracts were dried over MgSO4 and filtered. Concentration in vacuo afforded a yellow oil which was dissolved in 4 mL of anhydrous THF and heated to 70° C. with 300 mg Amberlyst-15 for about 45 min. During which time the uncyclized ester cyclized. The reaction mixture was filtered through a büchner funnel and concentrated in vacuo to give a pale yellow oil which spontaneously crystallized in a minimum amount of ethyl acetate. The white crystals (53% yield) were not further purified.

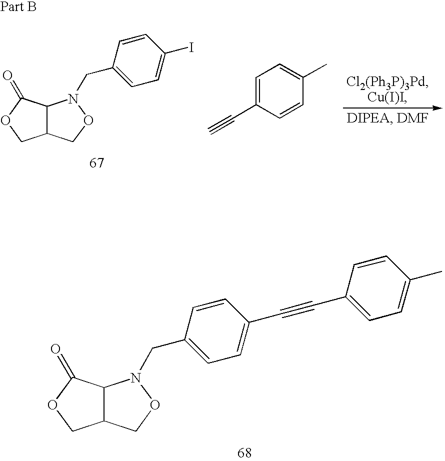

To a solution of 67 (1.0 eq., 0.522 mmol, 180 mg) dissolved in 2 mL anhydrous DMF in a round bottom flask under Argon was added dichlorobis(triphenylphosphine)palladium(II) (0.30 eq., 0.156 mmol, 110 mg) copper iodide (0.40 eq., 0.209 mmol, 40 mg) and the alkyne (2.0 eq., 1.04 mmol, 121 mg). Diisopropylethyl amine (4.0 eq., 2.09 mmol, 270 mg) was added slowly via syringe and the reaction mixture immediately turned dark brown. The reaction mixture was stirred at r.t. for 3½ h and was then quenched by adding water and diluted with ethyl acetate. The organic layer was washed 5× with water, 1× brine and dried over MgSO₄. Filtration and concentration in vacuo gave a black tar. The black solid was taken onto the next step without further purification.

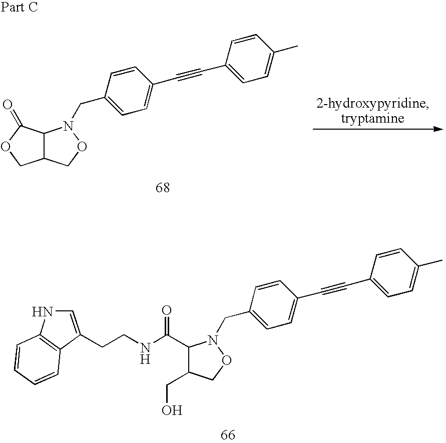

To a solution of 68 (1.0 eq., 0.510 mmol, 170 mg) in a 0.35 M 2-hydroxypyridine solution in anhydrous THF (3 mL) was added tryptamine (5 eq., 2.55 mmol, 408 mg). The reaction mixture was heated to 50° C. for 1 h. The reaction mixture was cooled to r.t., concentrated, and then purified by column chromatography (EtOAc) to give a pale yellow foam. 50% yield over two steps. MS (ESI(+)) m/e 494.32 (M+H)⁺.

### Example 53

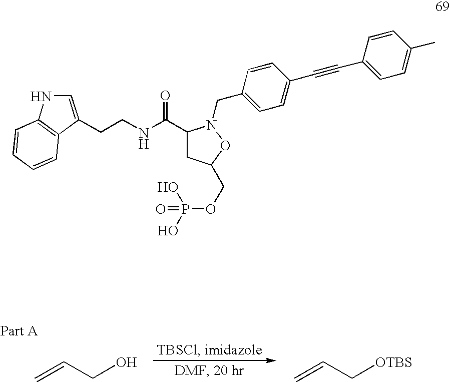

To a solution of allyl alcohol (1.0 eq., 8.61 mmol, 500 mg) in anhydrous DMF (20 mL) was added tert-butyl dimethylsilyl chloride (1.10 eq., 9.47 mmol, 1.43 g) and imidazole (11.1 eq., 95.7 mmol, 6.51 g). After stirring at r.t. for 20 h, water and DCM were added. The organic layer was separated and the aqueous layer was extracted 2× with DCM. The combined organic solution was washed with ice-cold 1 N HCl 3×, and water and dried over magnesium sulfate. Removal of solvent in vacuo afforded the silylated product as a colorless oil (96%).

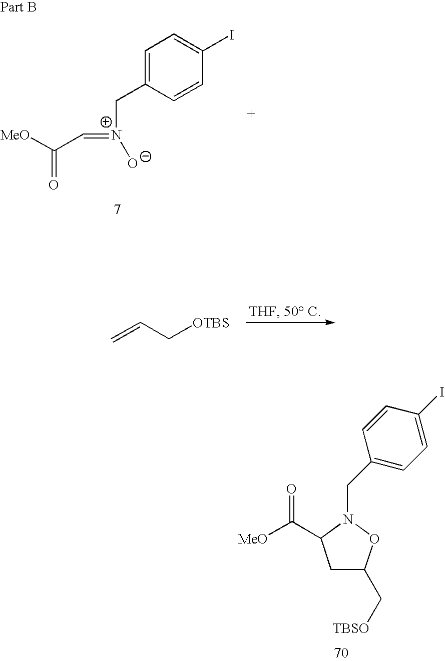

A reaction mixture containing nitrone methyl ester 7 (1.0 eq., 0.470 mmol, 150 mg) and TBS-protected allyl alcohol (1.0 eq., 0.470 mmol, 81 mg) in anhydrous THF (3 mL) was heated to 50° C. for 72 h and then concentrated in vacuo to give cyclized product (98%).

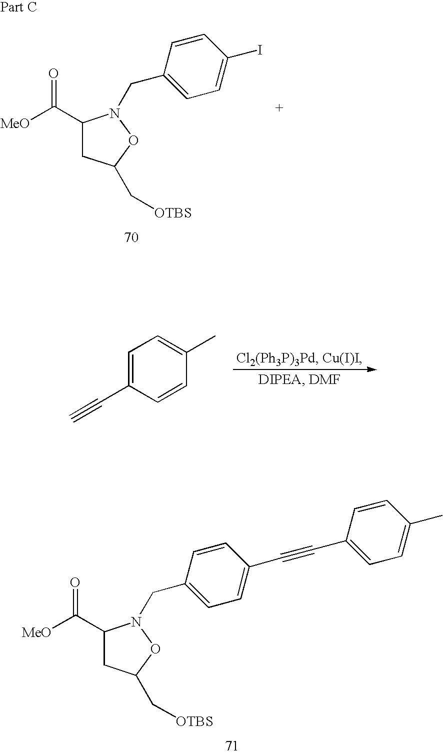

To a solution of 70 (1.0 eq., 0.470 mmol, 231 mg) dissolved in 3 mL anhydrous DMF in a round bottom flask under Argon was added dichlorobis(triphenylphosphine)palladium(II) (0.30 eq., 0.141 mmol, 99 mg) copper iodide (0.40 eq., 0.188 mmol, 36 mg) and the alkyne (2.0 eq., 0.940 mmol, 109 mg). Diisopropylethyl amine (4.0 eq., 1.88 mmol, 243 mg) was added slowly via syringe and the reaction mixture immediately turned dark brown. The reaction mixture was stirred at r.t. for 3½ h and was then quenched by adding water and diluted with ethyl acetate. The organic layer was washed 5× with water, 1× brine and dried over MgSO₄. Filtration and concentration in vacuo gave a black tar. The black solid was taken onto the next step without further purification.

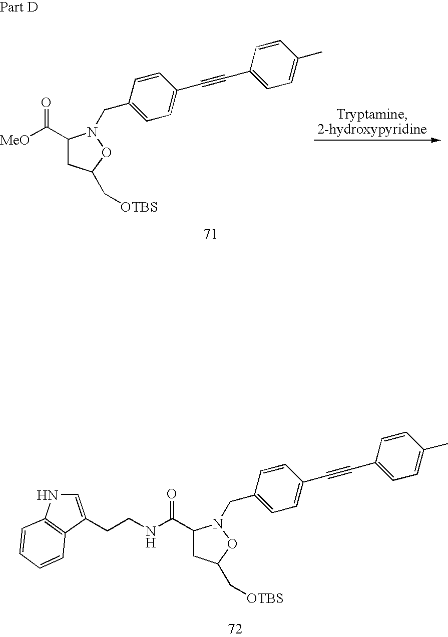

To a solution of 71 (1.0 eq., 0.479 mmol, 230 mg) in 0.35M 2-hydroxypyridine solution in anhydrous THF (5 mL) was added tryptamine (5 eq., 2.40 mmol, 384 mg). The reaction mixture was heated to 50° C. for 72 h concentrated in vacuo and purified by flash chromatography (ethyl acetate) (64% over 2 steps).

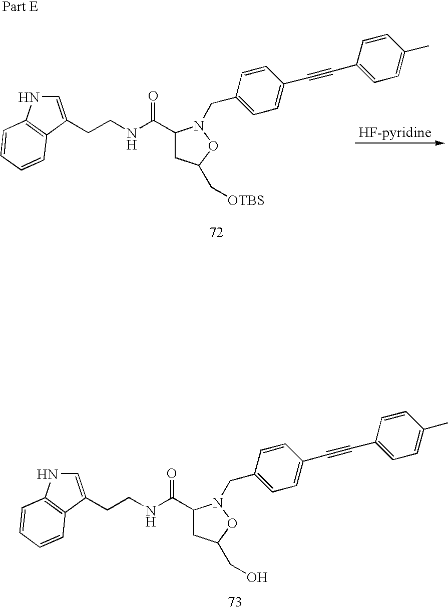

To a solution of 72 (1.0 eq., 0.041 mmol, 25 mg) in anhydrous THF (1 mL) at 0° C. was added HF-pyridine (10 eq., 58 μL) and the reaction mixture was allowed to stir for 2 h. The reaction mixture was quenched by careful addition of 3 mL of TMSOMe and then concentrated in vacuo to give the crude product. The product was purified by column chromatography. 90% yield.

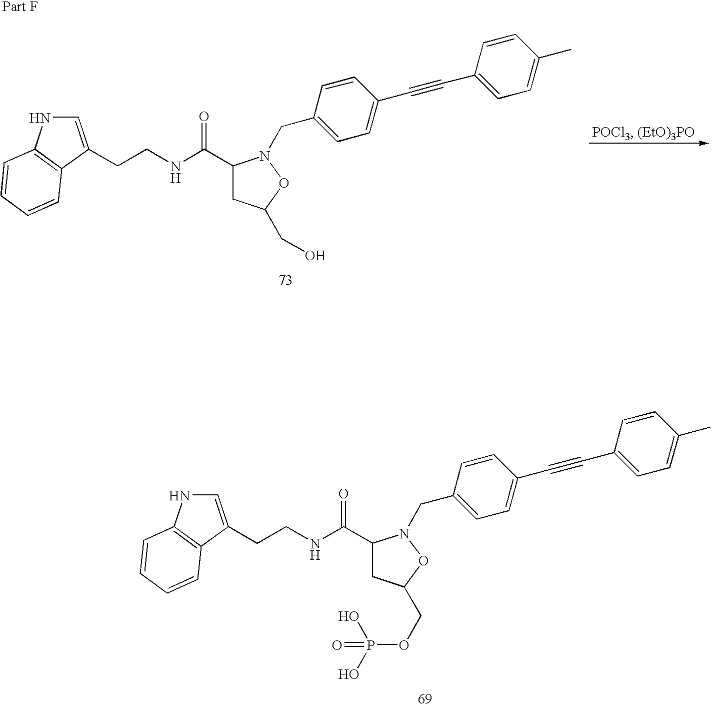

To a solution of phosphorus oxychloride (2.5 eq., 0.051 mmol, 8.5 mg) and triethyl phosphate (2.5 eq., 0.051 mmol, 9.2 mg) at 0° C. was added compound 73 in a minimum amount of triethylphosphate (150 μL), enough to dissolve the starting material. The reaction mixture was stirred for about 3 h warming to rt. The reaction mixture was diluted with water, and the aqueous solution was adjusted to pH 1.5 with a 1N NaOH sodium hydroxide solution and warmed at 70° C. for about 1 h. The resulting solution was neutralized with more sodium hydroxide and then concentrated on the genevac to afford a yellow oil and white solid. The yellow oil contained the product and the white solid was shown to be phosphate salts by LCMS. The crude mixture was purified by reverse-phase (C₈) HPLC with ammonium formate as buffer. 98% yield.

### Example 54

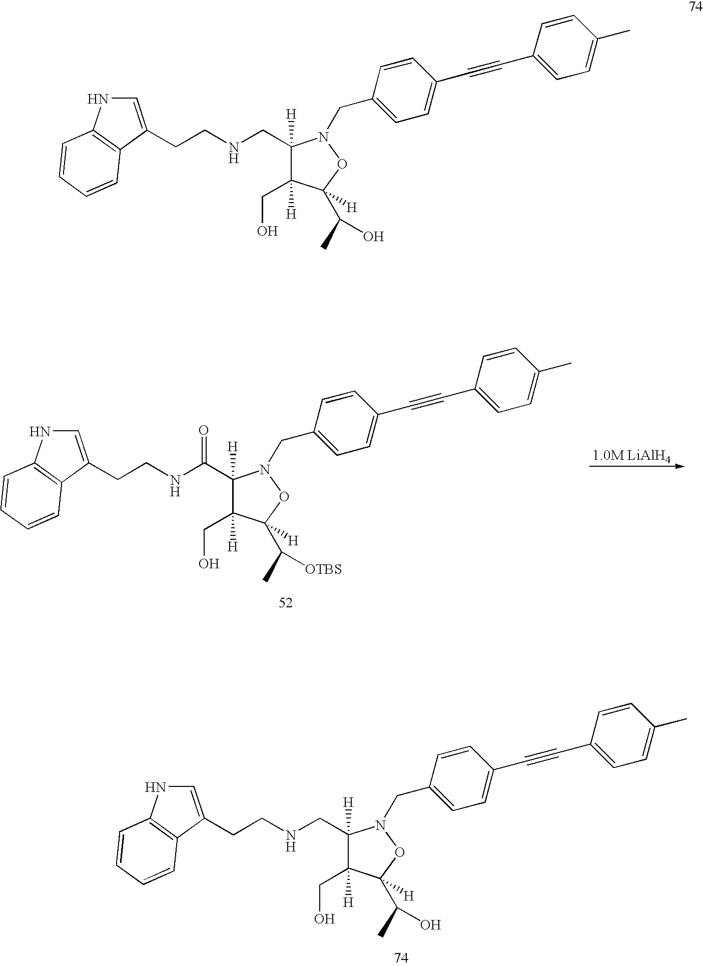

To a 1.0M solution of LAH in THF (1.0 eq., 0.038 mmol, 38 μL) at −78° C. was added a solution of compound 52 (1.0 eq., 0.038 mmol, 25 mg) in anhydrous THF (250 μL) drop wise via syringe. The reaction mixture was then warmed to rt and then slowly heated to reflux temperature (67° C.) for about 4 h. The reaction mixture was cooled to rt and then slowly quenched with ethyl acetate. To the mixture was added aq. Na₂SO₄, and then the mixture was extracted with ethyl acetate. The extract was dried over Na₂SO₄, concentrated in vacuo, and purified by column chromatography (4:1) CHCl₃/MeOH. 40% yield. MS (ESI(+)) m/e 524.20 (M+H)⁺.

### Example 55

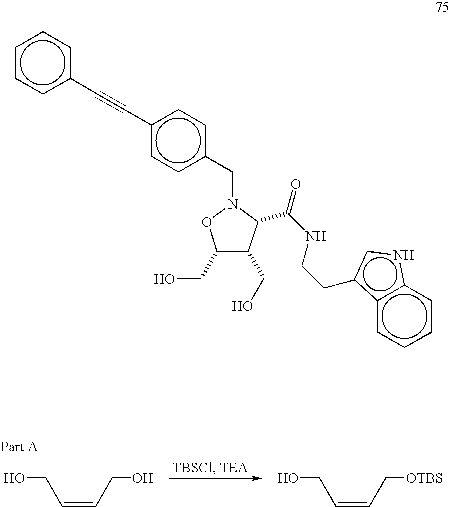

To a solution of but-2-ene-1,4-diol (6.53 mL, 80 mmol, 1 eq) in dry DCM (250 mL) was added triethylamine (23.3 mL, 2.1 eq.). The solution was stirred for 10 min and then a solution of TBS-Cl (14.4 g, 95 mmol, 1.2 eq) in dry DCM (67.8 mL) was added slowly. The reaction was stirred at rt for 3.5 h and then concentrated. The resulting residue was purified (9:1 HEA) to afford the desired product (5.54 g, 80 mmol).

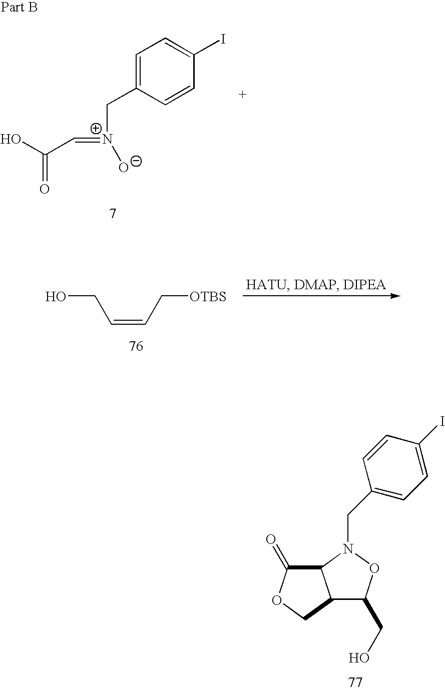

To a solution of 7 (0.3 g, 1 mmol, 1 eq) in dry DCM (3.43 mL) was added 76 (0.2 g, 1 mmol, 1 eq), HATU (0.72 g, 2 mmol, 2 eq), and DMAP (0.18 g, 1.5 mmol, 1.5 eq). The reaction solution was cooled to 0° C. and DIPEA (0.254 mL, 1.5 mmol, 1.5 eq) was added slowly. The solution was stirred at ⁰° C. for 1 h, warmed to rt, and stirred for another 2 h. The reaction solution was taken up in 1:1 DCM: 5% NaHCO₃ (60 mL). The layers were separated. The inorganic layer was extracted with EtOAc (2×30 mL). The organic layers were collected, dried over magnesium sulfate, filtered, and concentrated under reduced pressure. The resulting residue was dried and then dissolved in dry THF (1.72 mL). Amberlyst-15 (0.3 g) was added and the solution was heated to 70° C. After 2 h, the reaction was filtered and concentrated. The residue was purified (1:9 HEA, 0:10 HEA) to afford 0.244 g of the desired product.

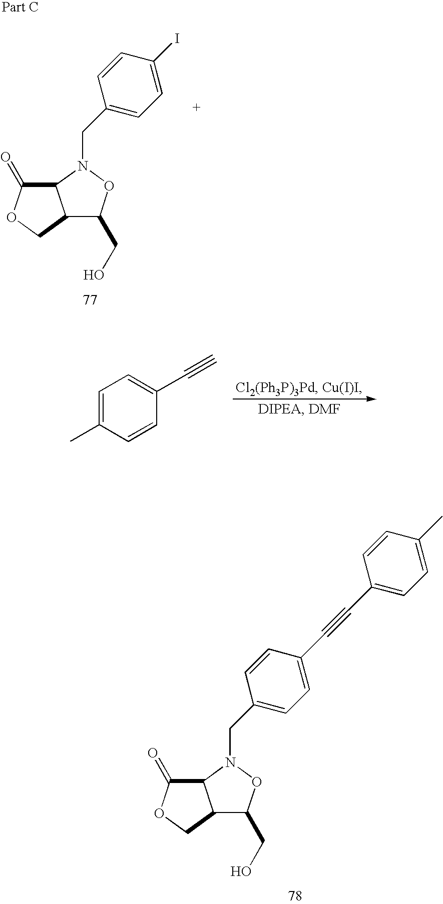

To a solution of 77 (0.244 g, 0.65 mmol, 1 eq) in dry DMF (1.3 mL) was added dichlorobis(triphenylphosphine)palladium(II) (0.14 g, 0.2 mmol, 0.3 eq), copper(I) iodide (0.05 g, 0.26 mmol, 0.4 eq), and 1-ethynyl-4-methyl-benzene (0.165 mL, 1.3 mmol, 2 eq). DIPEA (0.453 mL, 2.6 mmol, 4 eq) was added slowly to the solution and the reaction was stirred at rt for 3.5 h. The reaction was quenched with water and diluted with EtOAc. The organic layer was washed with water (5×) and brine and was then dried over magnesium sulfate, filtered and concentrated. The residue was dried to afford 0.403 g, of the crude product.

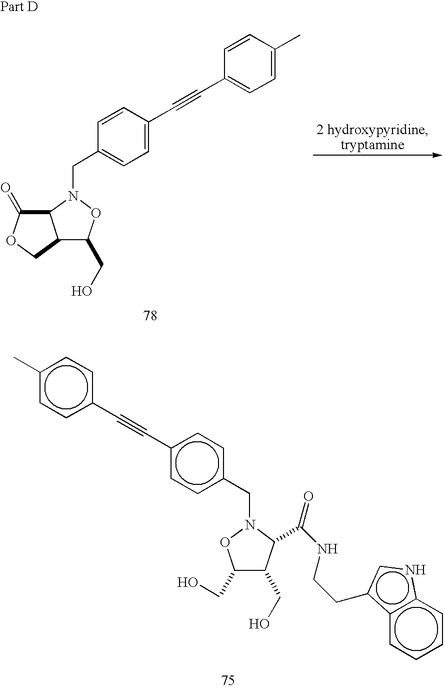

To a solution of 78 (0.403 g, 1 mmol) in 2-hydroxypyridine solution (6 mL, 2.0 M in dry THF) was added tryptamine (0.9 g, 6 mmol, 6 eq). The reaction was stirred at 50° C. for 3 h, the solution was concentrated, and the residue purified via flash chromatography to afford 0.203 g of the desired product. MS (ESI(+)) m/e 523.9 (M+H)⁺.

### Example 56

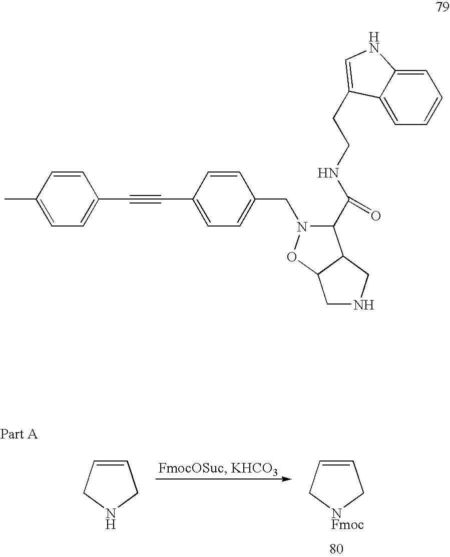

To a solution of 2,5-dihydro-1H-pyrrole (1.0 g, 10 mmol, 1 eq) in 3:1 THF:water (80 mL) at 0° C. was added potassium bicarbonate (20 g, 100 mmol, 10 eq). Fmoc-OSu (4.7 g, 10 mmol, 1 eq) was then added and the reaction was stirred overnight. The reaction solution was then taken up in water and the layers separated. The aqueous layer was washed with EtOAc (3×). The organic layers were collected, dried over sodium sulfate, filtered, and concentrated. The resulting yellow liquid was purified (3:1 HEA) to afford the desired product.

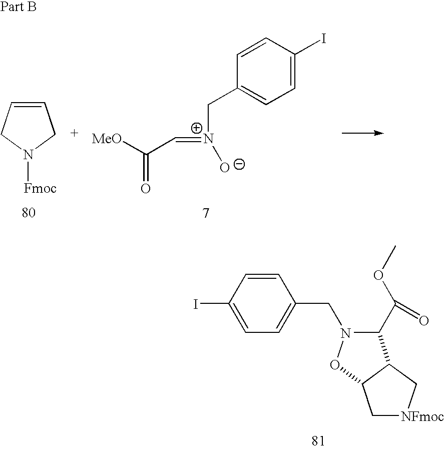

To a solution of nitrone methyl ester 7 (0.3 g, 1.02 mmol, 1 eq) in dry THF (3 mL) was added 80 (0.274 g, 1.03 mmol, 1 eq). The reaction solution was stirred at 60° C. for 5 days and then concentrated. The resulting residue was purified via column chromatography (2:1H EA then 1:1 HEA) to afford 0.332 g of the desired product.

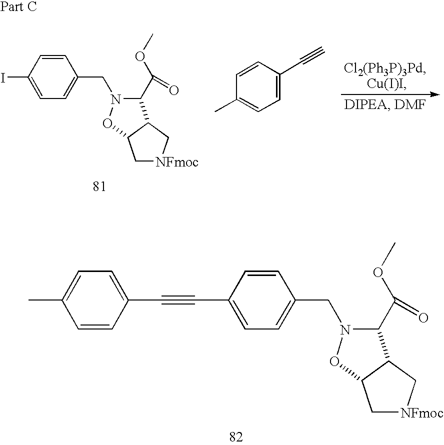

To a solution of 81 (0.332 g, 0.544 mmol, 1 eq) in dry DMF (2 mL) was added dichlorobis(triphenylphosphine)palladium(II) (0.115 g, 0.163 mmol, 0.3 eq), copper(I) iodide (0.04 g, 0.22 mmol, 0.4 eq), and 1-ethynyl-4-methyl-benzene (0.12 mL, 1.1 mmol, 2 eq). DIPEA (0.379 mL, 2.2 mmol, 4 eq) was added slowly to the solution and the reaction was stirred at rt for 2 h. The reaction was quenched with water and diluted with EtOAc. The organic layer was washed with water (5×) and brine and was then dried over magnesium sulfate, filtered and concentrated under reduced pressure. The resulting residue was purified via column chromatography (1:1 HEA) to afford 0.303 g, of the desired product.

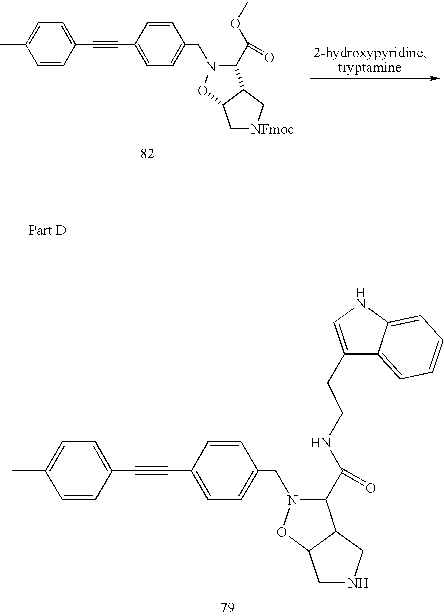

To a solution of 82 (0.303 g, 0.507 mmol, 1 eq) in 2-hydroxypyridine solution (5 mL, 2.0 M in dry THF) was added tryptamine (0.81 g, 5.07 mmol, 10 eq). The reaction was stirred at 50° C. for 3 h, the solution was concentrated, and the residue purified via column chromatography to afford 0.969 g of the desired product. MS (ESI(+)) m/e 505.5 (M+H)⁺.

### Example 57

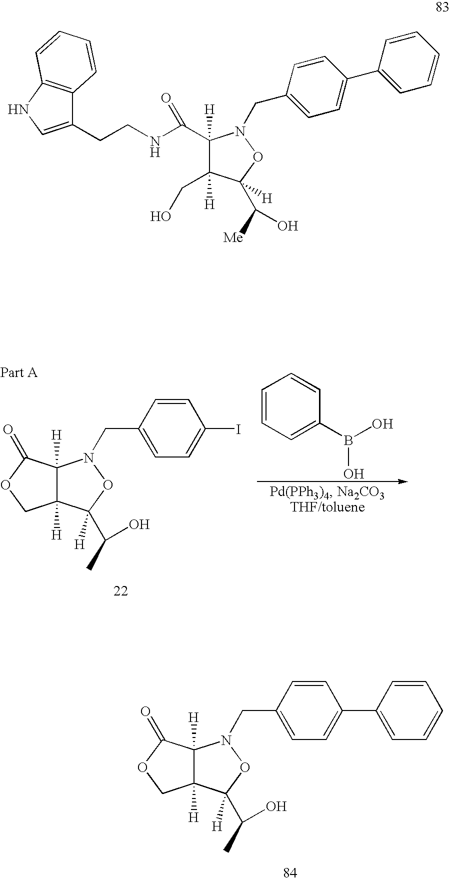

To a 25 mL round bottom flushed with argon was added isoxazolidine 22 (47 mg) followed by 3 mL toluene and 3 mL THF. To this solution was added phenyl boronic acid (59 mg), sodium carbonate (51 mg) and 0.4 mL distilled water. The solution was degassed with argon for 40 minutes followed by the addition of tetrakis(triphenylphosphine)palladium (14 mg). The solution was then heated to 70° C. for 4 h. The reaction was diluted with methylene chloride and washed with water and then with brine. The organics were dried over magnesium sulfate and concentrated in vacuo to yield 10.6 mg (26%) of a yellow oil.

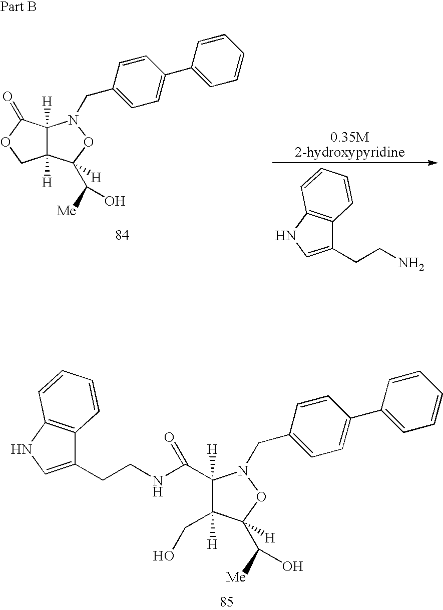

To a 20 mL scintillation flask containing biphenyl lactone 84 (10.6 mg) and tryptamine (30 mg) was added 0.35 M 2-hydroxypyridine (0.4 mL). The solution was then heated to 50° C. for 18 h. The crude reaction was then diluted with ethyl acetate (20 mL) and water (20 mL). The layers were separated and the organic layer was washed with brine (20 mL) and dried over magnesium sulfate. The organics were concentrated in vacuo and the product purified by preparative TLC (1000 microns, silica) by elution with 4:1 ethyl acetate/hexanes to yield 8 mg (50%) of a white film. MS (ESI(+)) m/e 499.9 (M+H)⁺.

### Example 58

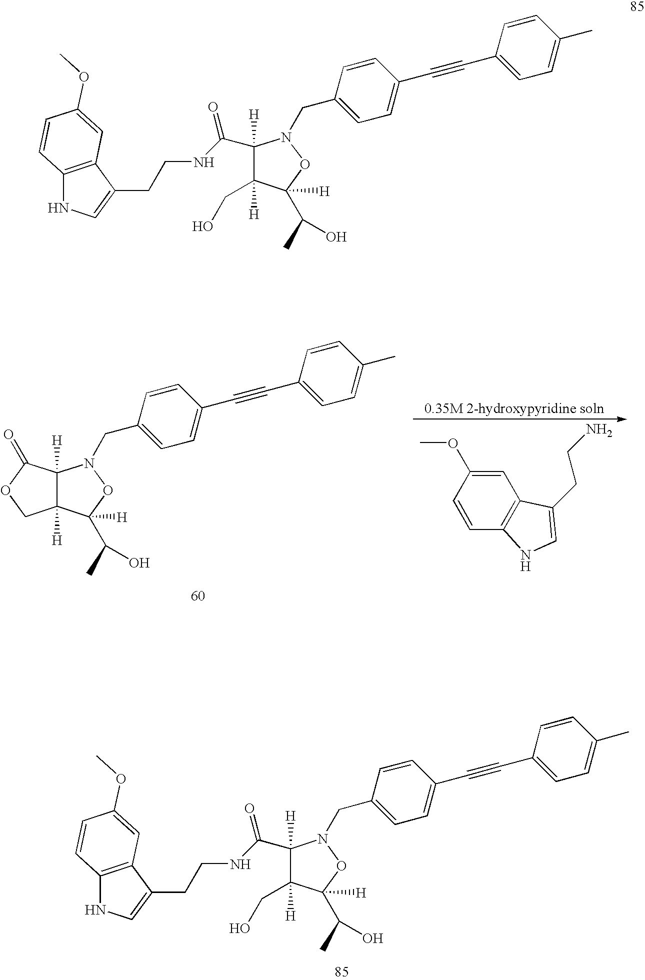

To a 20 mL scintillation vial was added isoxazolidine 60 (30 mg) and 5-methoxytrypamine (80 mg). Then 0.35M 2-hydroxypyridine in THF (0.5 mL) was added to the solids. The vial was capped and heated to 50° C. for 12 h. The crude reaction was then diluted with ethyl acetate (50 mL) and washed with 0.1 N HCl (50 mL) and brine (50 mL brine). The organic layer was dried sodium sulfate and then concentrated in vacuo to yield 38 mg (80%) of an orange oil. MS (ESI(+)) m/e 568.0 (M+H)⁺.

### Example 59

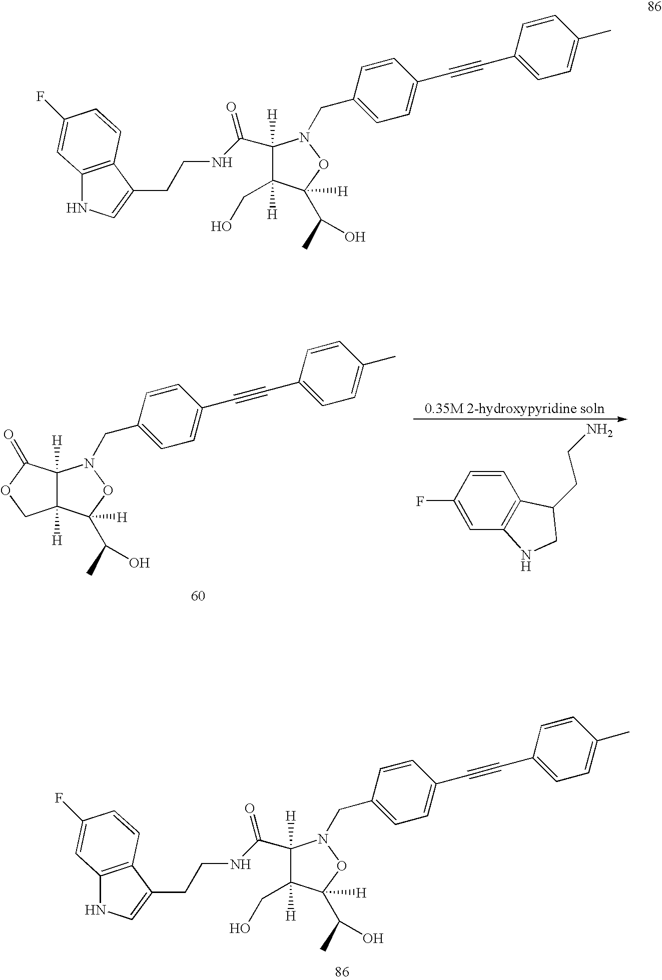

To a 20 mL scintillation vial was added isoxazolidine 60 (20 mg) and 6-fluorotyptamine (149 mg) followed by 0.35M 2-hydroxypyridine solution (1.0 mL). The solution was heated to 50° C. for 14 h and then the title compound was purified by loading directly on to a preparative TLC plate (1000 microns, silica) and eluted with 95:5 ethyl acetate/methanol to yield 26 mg (100%) of a pale yellow solid. MS (ESI(+)) m/e 556.0 (M+H)⁺.

### Example 60

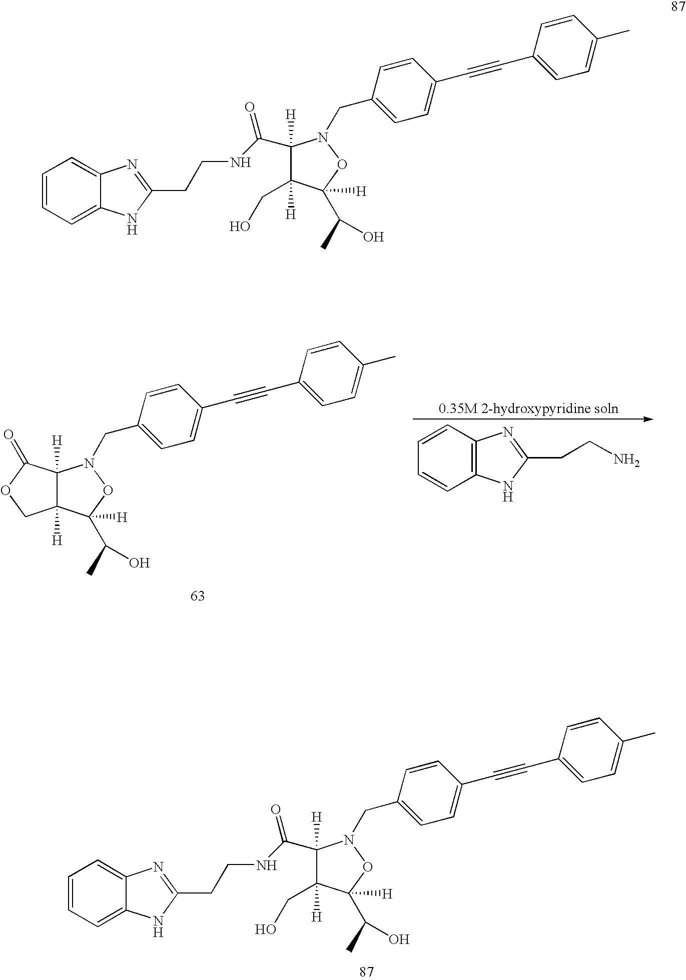

To a 20 mL scintillation vial was added isoxazolidine 60 (20 mg) and 1-H-benzimidazole-2-ethanamine (72.1 mg) followed by 0.35 M 2-hydroxypyridine solution (0.7 mL). The solution was heated to 50° C. for 19 h and then diluted with ethyl acetate (10 mL) and water (10 mL). The organic layer was separated and concentrated in vacuo. The title compound was purified by preparative TLC (1000 microns, silica) eluting with 95:5 ethyl acetate/methanol to yield 12.7 mg of the desired product as a solid. MS (ESI(+)) m/e 539.0 (M+H)⁺.

### Example 61

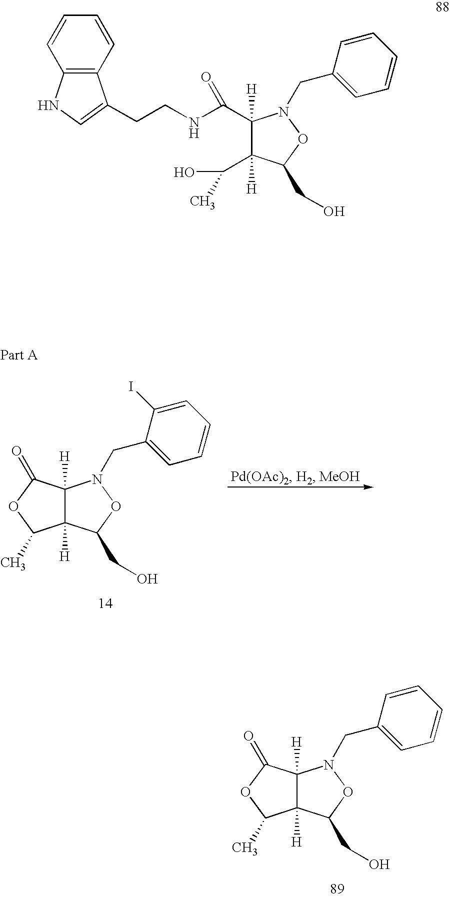

To isoxazolidine 14 (606 mg) and palladium acetate (46 mg) was added methanol (30 mL) under argon. The flask was evacuated and flushed with hydrogen three times and stirred under 1 atm of hydrogen. After 6 h, more palladium acetate was added (30 mg) and stirring continued under 1 atm hydrogen atmosphere. After 21.5 total h more palladium acetate was added (60 mg). Finally, after 36 total h, more palladium acetate was added (60 mg). After another 132 total h, ammonium formate (100 mg) was added and after another 24 h ammonium formate (200 mg) and palladium acetate (20 mg) was added while continuing to stir under 1 atm hydrogen balloon. After 24 h following this final addition, the reaction was filtered through celite and washed with methanol. The organics were concentrated in vacuo. The solids were dissolved in water (100 mL) and ethyl acetate (100 mL). The organic layer was separated, dried with sodium sulfate and concentrated in vacuo to yield 316.4 mg (90%) of a pale yellow crystalline solid.

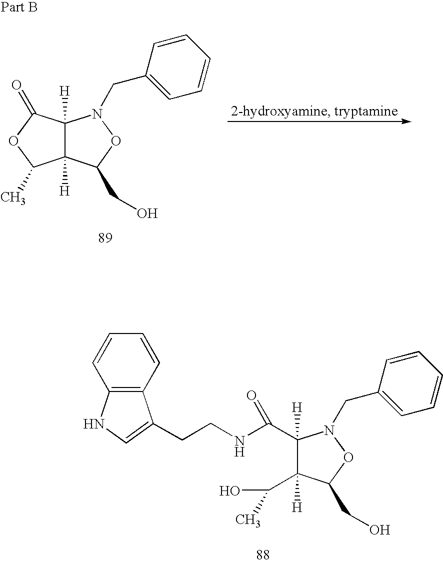

To a 20 mL scintillation vial was added isoxazolidine 89 (15 mg) and tryptamine (46 mg) followed by 0.35 M 2-hydroxypyridine solution (0.5 mL). The solution was heated to 50° C. for 40 minutes and then cooled to 35° C. and allowed to stir for 16 h. The title compound was purified by preparative TLC (1000 microns, silica) by elution with ethyl acetate to yield 11.3 mg (47%) of the desired product as an oil. MS (ESI(+)) m/e 424.8 (M+H)⁺.

### Example 62

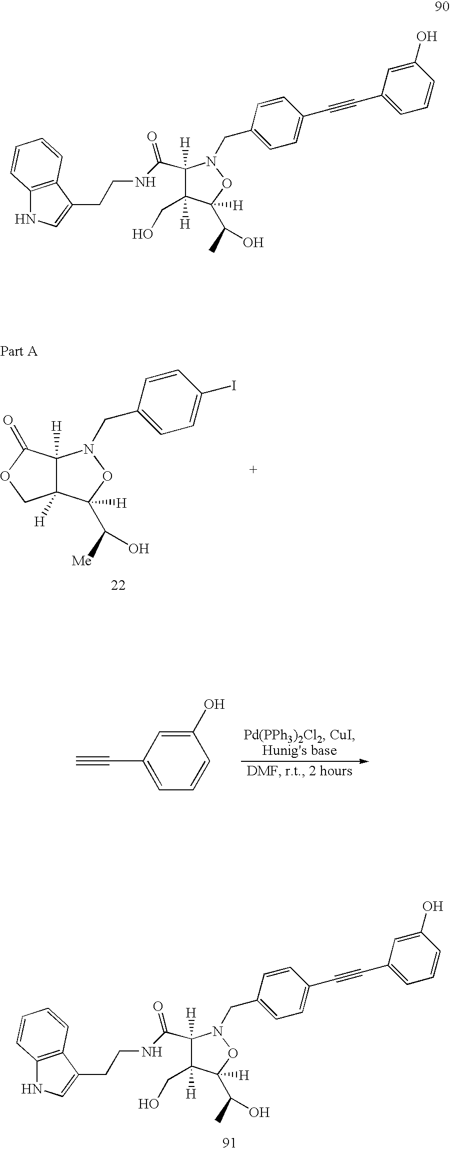

To a 10 mL round bottom was added isoxazolidine 22 (50 mg), dichlorobis(triphenylphosphine)palladium (30 mg), copper iodide (10 mg) and 3-hydroxyphenylacetylene (30 mg). The flask was flushed with argon and then DMF was added (2 mL) followed by diisopropylethylamine (70 μL). The reaction was stirred for 6 h and then diluted with ethyl acetate (50 mL), washed with 1 N HCl (50 mL) and brine (50 mL). The organic layer was dried with sodium sulfate and concentrated in vacuo. The title compound was purified by preparative TLC (1000 microns, silica) by elution with 3:1 ethyl acetate/hexanes to yield 48 mg (98%).

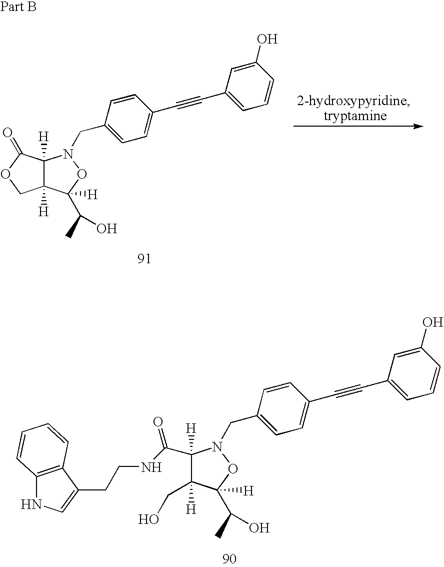

To a 20 mL scintillation vial was added isoxazolidine 91 (48 mg) and tryptamine (100 mg) followed by 0.35 M 2-hydroxypyridine solution (0.5 mL). The solution was heated to 50° C. for 12 h and then diluted with ethyl acetate (50 mL). The organics were washed with 0.1 N HCl (50 mL) and brine (50 mL) and then dried with sodium sulfate. The organic layers were concentrated in vacuo to yield 60 mg of the desired product as a red oil. MS (ESI(+)) m/e 540.0 (M+H)⁺.

### Example 63

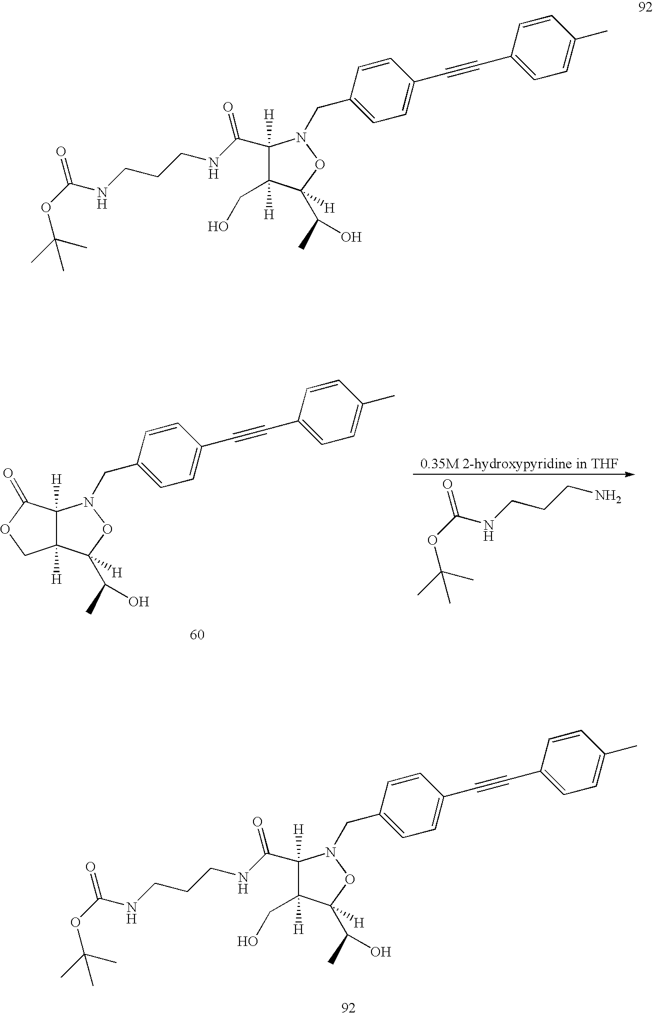

To a 20 mL scintillation vial was added isoxazolidine 60 (17 mg) and tert-butyl N-(3-aminopropyl) carbamate (100 mg) followed by 0.35 M 2-hydroxypyridine solution (0.9 mL). The solution was heated to 50° C. for 11 h and then diluted with ethyl acetate (50 mL). The organics were washed with 0.1 N HCl (50 mL) and brine (50 mL) and then dried with sodium sulfate. The organic layers were concentrated in vacuo. The title compound was purified by preparative TLC (1000 microns, silica) eluting with 4:1 ethyl acetate/methanol to yield 22.3 mg of the title compound. MS (ESI(+)) m/e 553.0 (M+H)⁺.

### Example 64

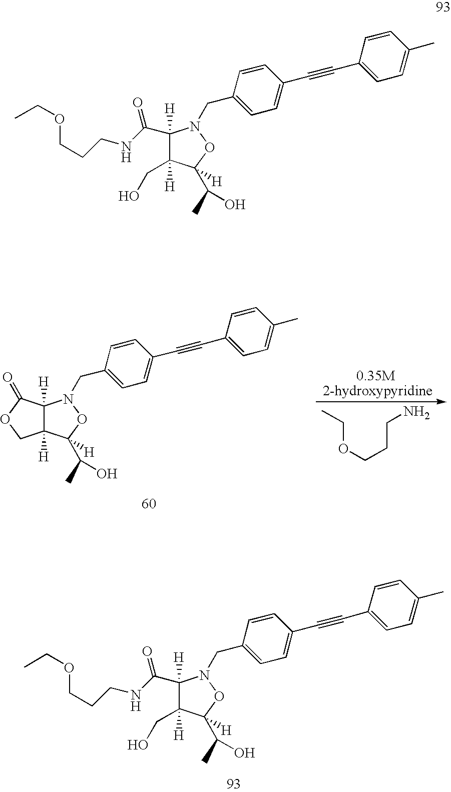

To a 20 mL scintillation vial was added isoxazolidine 60 (20 mg) and 3-ethoxypropylamine (32 mg) followed by 0.35M 2-hydroxypyridine solution (1.0 mL). The solution was heated to 50° C. for 14 h and then purified the title compound by loading directly on to a preparative TLC (1000 microns, silica) and eluting with 95:5 ethyl acetate/methanol to yield 15.3 mg (60%) of an eggshell colored solid. MS (ESI(+)) m/e 480.9 (M+H)⁺.

### Example 65

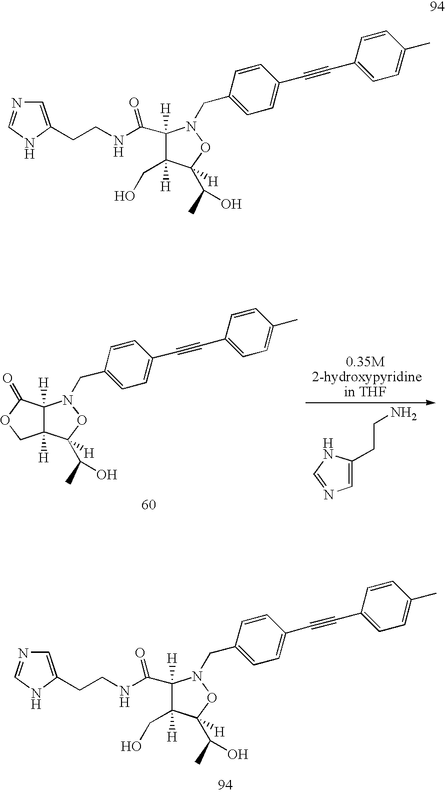

To a 20 mL scintillation vial was added isoxazolidine 60 (20 mg) and histamine (29 mg) followed by 0.35 M 2-hydroxypyridine solution (1.0 mL). The solution was heated to 50° C. for 14 h and then the title compound was purified by loading directly on to a preparative TLC (1000 microns, silica) and eluting with 95:5 ethyl acetate/methanol to yield 13 mg (50%) of the title compound. MS (ESI(+)) m/e 489.9 (M+H)⁺.

### Example 66

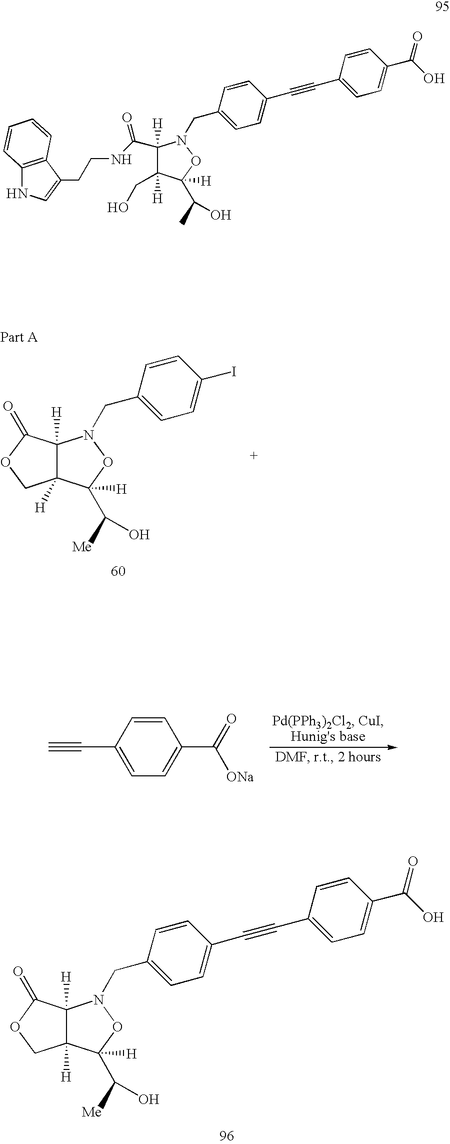

To a 10 mL round bottom was added isoxazolidine 60 (50 mg), dichlorobis(triphenylphosphine)palladium (30 mg), copper iodide (10 mg) and (4-carboxyphenyl)acetylene sodium salt (40 mg). The flask was flushed with argon and then DMF was added (2 mL) followed by diisopropylethylamine (70 μL). The reaction was stirred for 6 h and then diluted with ethyl acetate (50 mL) and washed with 1N HCl (50 mL), and brine (50 mL). The organic layer was dried with sodium sulfate and concentrated in vacuo. The title compound was purified by preparative TLC (1000 microns, silica) by elution with ethyl acetate to yield 17.8 mg (34%) of the title compound.

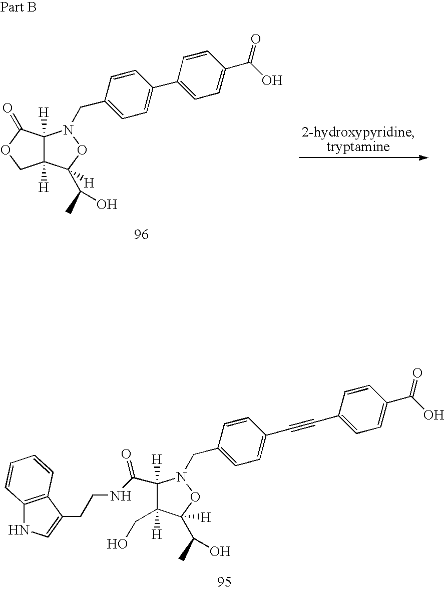

To a 20 mL scintillation vial was added isoxazolidine 96 (17.8 mg) tryptamine (35 mg) followed by 0.35 M 2-hydroxypyridine solution (0.5 mL). The solution was heated to 50° C. for 12 h and the crude reaction diluted with ethyl acetate (50 mL). The organic layer was washed with 0.1 N HCl (50 mL), brine (50 mL), dried with sodium sulfate and concentrated in vacuo to yield 20 mg (80%) of the title compound as an orange oil. MS (ESI(+)) m/e 568.0 (M+H)⁺.

### Example 67

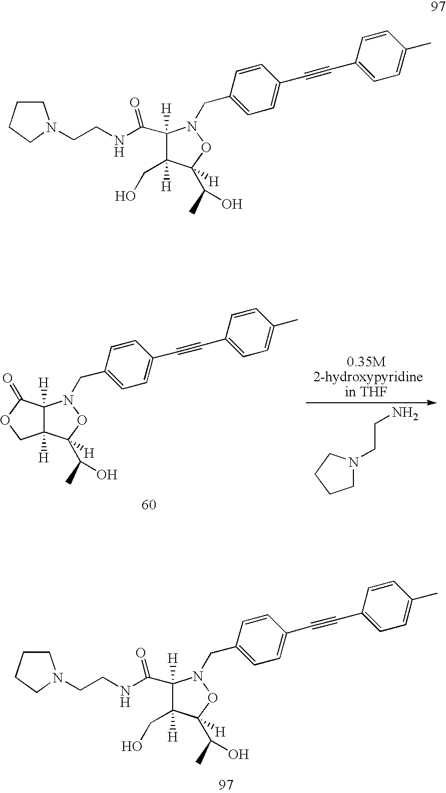

To a 20 mL scintillation vial was added isoxazolidine 60 (20 mg) N-(2-aminoethyl)pyrrolidine (34 mg) followed by 0.35 M 2-hydroxypyridine solution (1.0 mL). The solution was heated to 50° C. for 14 h and then the title compound was purified by loading directly on to a preparative TLC (1000 microns, silica) and eluting with 95:5 ethyl acetate/methanol to yield 26 mg (100%) of the title compound as a clear oil. MS (ESI(+)) m/e 491.9 (M+H)⁺.

### Example 68

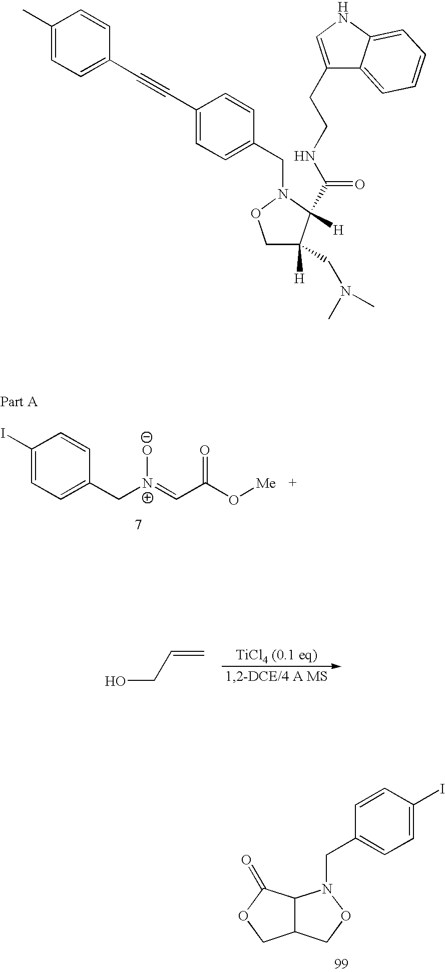

Nitrone methyl ester 7 (0.5 g, 2 mmol), allyl alcohol (0.1 g, 2 mmol) were dissolved in 4 mL of 1,2-dichloroethane (4 mL). 4 A molecular sieves (0.4 g) and TiCl₄ (1.0 M in DCM, 0.1 eq, 0.16 mL) were added and mixture was heated to 55° C. under a nitrogen atmosphere. The reaction was heated for 4 h and then stirred at rt for 14 h. The mixture was then diluted with EtOAc (15 mL), washed with 1% HCl in water, brine, and dried over MgSO₄. The resulting mixture was then filtered and concentrated under reduced pressure. The resulting mass was recrystallized from MTBE/hexane to yield 337 mg of the desired product.

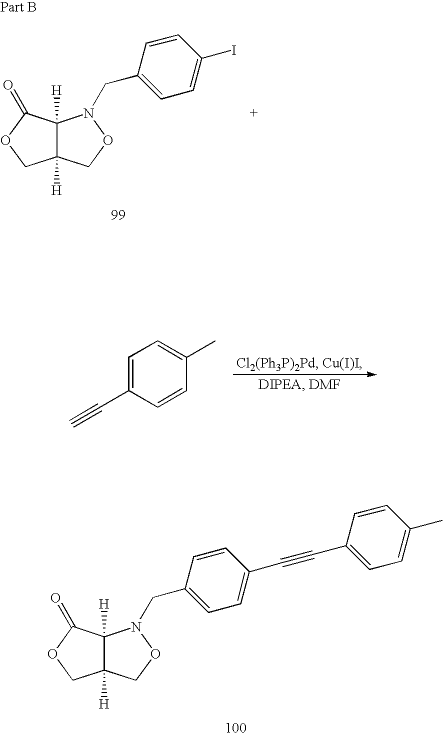

Compound 100 was synthesized according to the procedure described in synthesis of compound 51. Yield 90%.

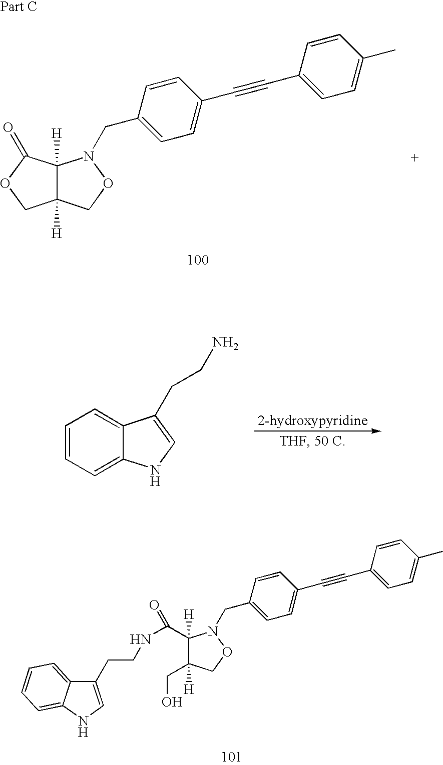

Compound 100 (0.5 mmol, 1 eq.) was dissolved in THF (5 mL). 2-Hydroxypyridine (0.24 g, 2.5 mmol) and tryptamine (0.4 g, 2.5 mmol) were added and the resulting solution was heated to 60° C. under nitrogen for 5 days. The reaction solution was then cooled to rt and diluted with EtOAc (15 mL). The organic solution was then washed with water, brine, dried over MgSO₄ and concentrated under reduced pressure. The crude was purified via flash chromatography to yield 179 mg of the desired product.

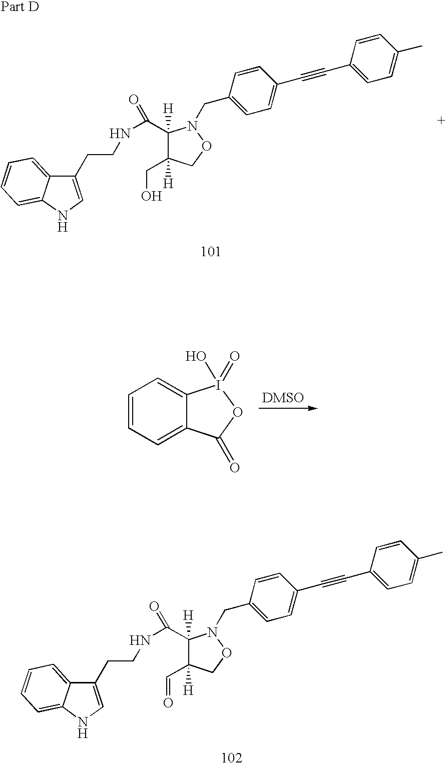

Compound 101 (0.24 g, 0.5 mmol) was dissolved in anhydrous DMSO (2 mL). IBX (0.28 g, 1.0 mmol) was added in one portion under nitrogen and the reaction was allowed to stir at RT for 24 h. The reaction mixture was then diluted with EtOAc (15 mL) and then extracted with water (3×10 mL), dried over MgSO₄ and concentrated under reduced pressure. The crude was purified via column chromatography to afford 160 mg of the desired product.

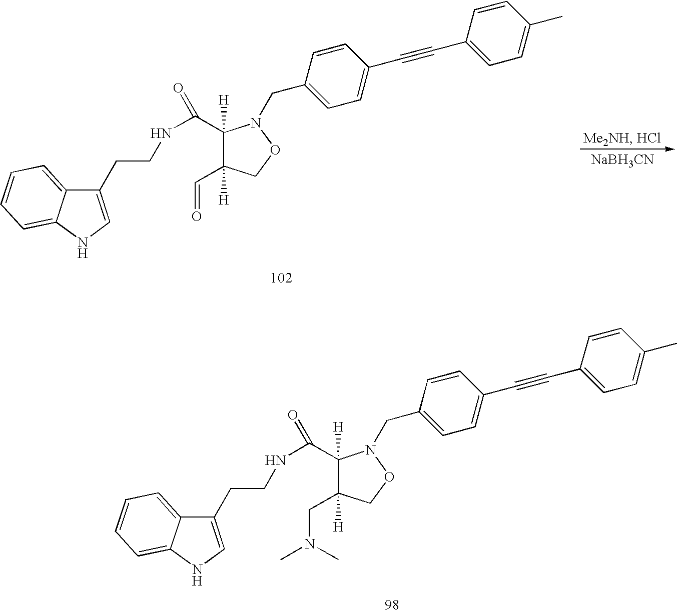

Compound 102 (50 mg, 0.1 mmol) was dissolved in MeOH/DCM (1:12 mL). Me₂NH.HCl (41 mg, 0.5 mmol, 5 equiv.) and NaBH₃CN (20 mg, 0.3 mmol, 3 equiv.) were added and the reaction was stirred under nitrogen at rt until all starting material had been consumed as indicated by TLC. The reaction mixture was then diluted with EtOAc (15 mL) and quenched with saturated sodium bicarbonate. The organic layer was collected, washed with water, dried over MgSO₄ and concentrated under reduced pressure. The crude was purified via column chromatography to afford 20 mg of the desired product. MS (ESI(+)) m/e 521.2 (M+H)⁺.

### Example 69

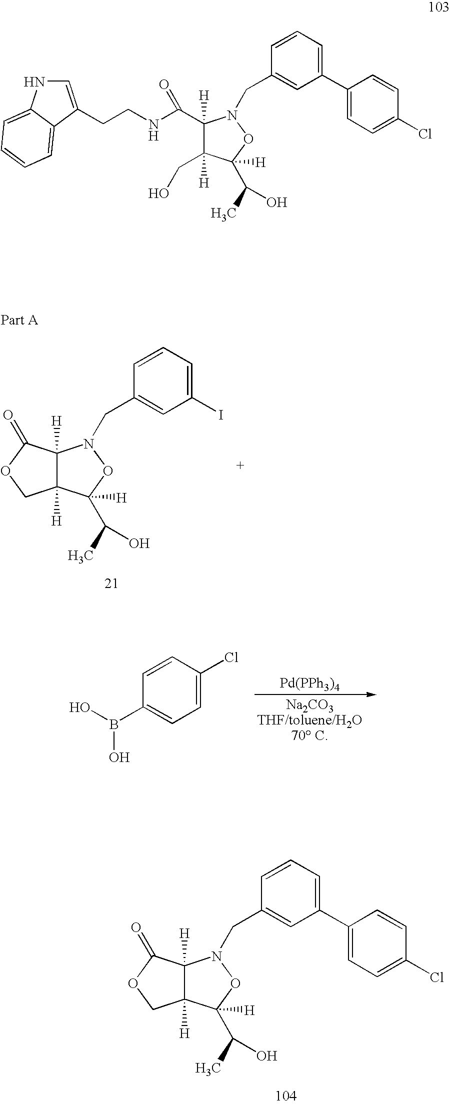

Meta-Iodo isoxazolidine core 21 (0.10 g, 0.26 mmol, 1.0 equiv) was dissolved in 5 mL of THF and 5 mL toluene (0.03 M) under a nitrogen atmosphere with stirring. To this solution was added 4-chlorophenylboronic acid (0.16 g, 1.0 mmol, 4.0 equiv) and Na₂CO₃ (0.11 g, 1.0 mmol, 4.0 equiv). The solution was stirred for 1 min followed by the addition of 1 mL H₂O and Pd(PPh₃)₄ (0.030 g, 0.026 mmol, 0.10 equiv). The yellow solution was then heated to 70° C. for 2 h until TLC showed the consumption of starting material. The mixture was added to a separatory funnel with 20 mL CH₂Cl₂ and 20 mL water and the layers were separated. The aqueous layer was extracted with an additional 10 mL CH₂Cl₂ and the combined organics were washed with 10 mL brine, dried with MgSO₄ and concentrated. The crude material was purified by silica gel chromatography to yield biaryl isoxazolidine compound 104 as an off-white solid (0.070 g, 0.19 mmol, 73% yield).

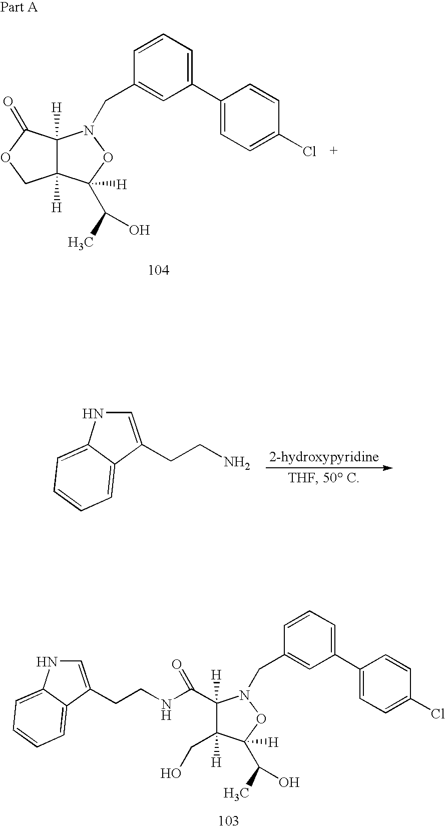

Meta-Iodo isoxazolidine biaryl 104 (0.70 g, 0.19 mmol, 1.0 equiv) was dissolved in 1.7 mL THF (0.09M) under a nitrogen atmosphere with stirring. To this solution was added 2-hydroxypyridine (0.071 g, 0.75 mmol, 4.0 equiv) and tryptamine (0.15 g, 0.94 mmol, 5.0 equiv). The solution was stirred at 50° C. for 2 h, quenched with 0.1 N HCl and extracted with ethyl acetate. The aqueous layer was extracted an additional time with ethyl acetate and the combined organics were washed with brine, dried with MgSO₄, and concentrated. The crude material was purified by silica gel chromatography to yield the desired product as a brown solid (0.050 g, 0.01 mmol, 50% yield). MS (ESI(+)) m/e 533.9 (M+H)⁺.

### Example 70

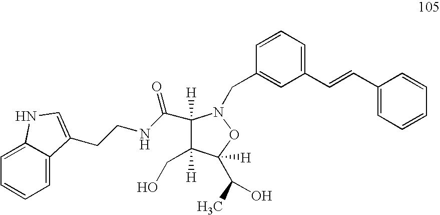

Compound 105 was synthesized according to the procedure described in the synthesis of Example 69 using trans-2-phenylvinylboronic acid in place of 4-chlorophenylboronic acid. Overall yield: 25-50%. MS (ESI(+)) m/e 526.3 (M+H)⁺.

### Example 71

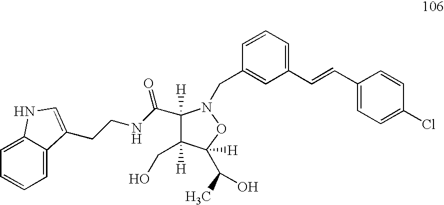

Compound 106 was synthesized according to the procedure described in the synthesis of Example 69 using trans-2-(4-chlorophenyl) vinylboronic acid in place of 4-chlorophenylboronic acid. Overall yield: 25-50%. MS (ESI(+)) m/e 560.29 (M+H)⁺.

### Example 72

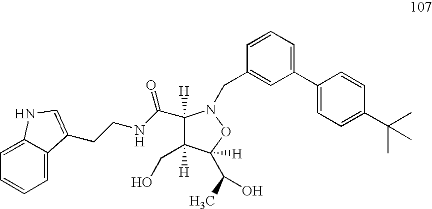

Compound 69 was synthesized according to the procedure described in the synthesis of Example 103 using 4-t-butylphenylboronic acid in place of 4-chlorophenylboronic acid. Overall yield: 25-50%. MS (ESI(+)) m/e 556.0 (M+H)⁺.

### Example 73

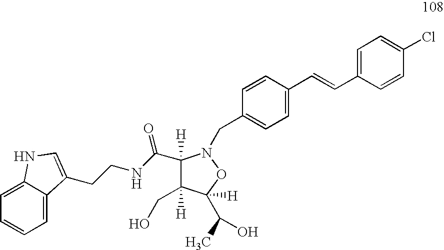

Compound 108 was synthesized according to the procedure described in the synthesis of Example 69 using isoxazolidine core 22 in place of isoxazolidine core 21, and trans-2-(4-chlorophenyl) vinylboronic acid in place of 4-chlorophenylboronic acid. Overall yield: 25-50%. MS (ESI(+)) m/e 560.0 (M+H)⁺.

### Example 74

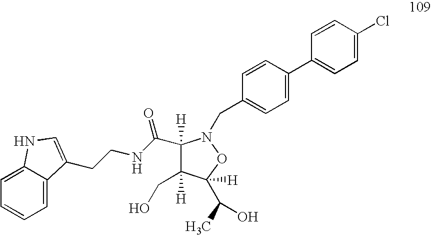

Compound 109 was synthesized according to the procedure described in the synthesis of Example 69 using isoxazolidine core 22 in place of isoxazolidine core 21, and 4-chlorophenylboronic acid. Overall yield: 25-50%.

### Example 75

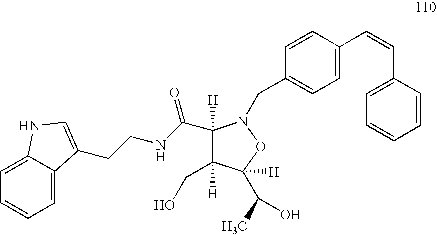

Compound 110 was synthesized according to the procedure described in the synthesis of Example 69 using isoxazolidine core 22 in place of isoxazolidine core 21, and cis-2-phenylvinylboronic acid in place of 4-chlorophenylboronic acid. Overall yield: 25-50%. MS (ESI(+)) m/e 525.9 (M+H)⁺.

### Example 76

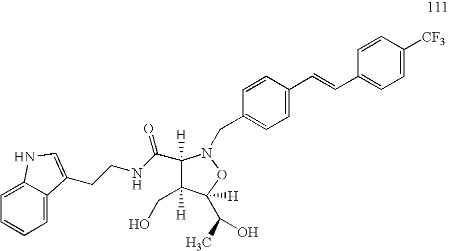

Compound 111 was synthesized according to the procedure described in the synthesis of Example 69 using isoxazolidine core 22 in place of isoxazolidine core 21, and trans-2-(4-trifluoromethylphenyl) vinylboronic acid in place of 4-chlorophenylboronic acid. Overall yield: 25-50%. MS (ESI(+)) m/e 594.1 (M+H)⁺.

### Example 77

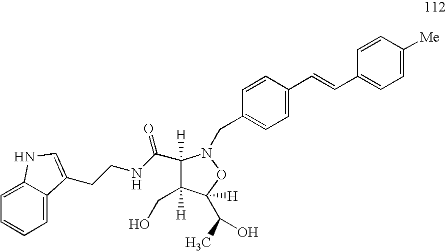

Compound 112 was synthesized according to the procedure described in the synthesis of Example 69 using isoxazolidine core 22 in place of isoxazolidine core 21, and trans-2-(4-methylphenyl) vinylboronic acid in place of 4-chlorophenylboronic acid. Overall yield: 25-50%.

### Example 78

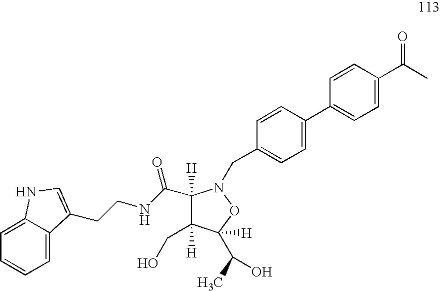

Compound 69 was synthesized according to the procedure described in the synthesis of Example 103 using isoxazolidine core 22 in place of isoxazolidine core 21, and 4-acetylbenzeneboronic acid in place of 4-chlorophenylboronic acid. Overall yield: 25-50%. MS (ESI(+)) m/e 542.0 (M+H)⁺.

### Example 79

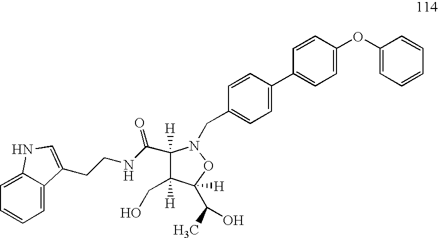

Compound 114 was synthesized according to the procedure described in the synthesis of Example 69 using isoxazolidine core 22 in place of isoxazolidine core 21, and 4-phenoxyphenylboronic acid in place of 4-chlorophenylboronic acid. Overall yield: 25-50%.

### Example 80

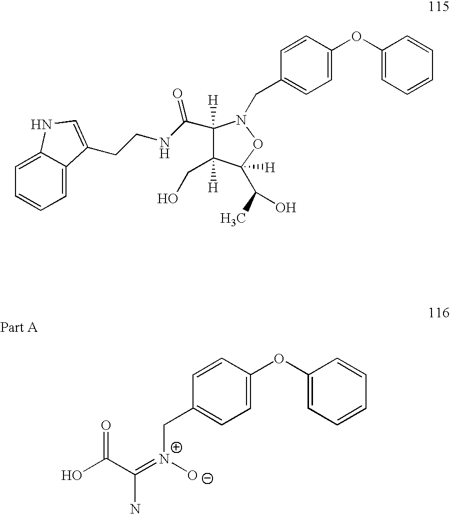

Nitrone carboxylic acid 116 was synthesized according to the procedure described in the synthesis of nitrone carboxylic acid 4 using 4-phenoxybenzaldehyde in place of 4-iodoaldehyde. 10% overall yield.

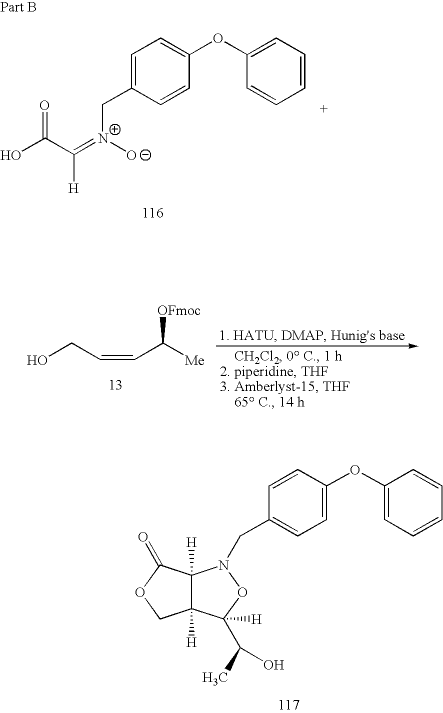

Nitrone carboxylic acid 116 (0.22 g, 0.81 mmol, 1.2 equiv), Fmoc allylic alcohol 13 (0.22 g, 0.68 mmol, 1.0 equiv), HATU (0.51 g, 1.4 mmol, 2.0 equiv), and DMAP (0.13 g, 1.0 mmol, 1.5 equiv) were combined with 3 mL CH₂Cl₂ under nitrogen atmosphere. The cloudy solution was then cooled in an ice-bath for 20 min. Diisopropylethylamine (0.18 mL, 1.0 mmol, 1.5 equiv) was then added drop wise over 5 min and the reaction was maintained at 0° C. for 1 h. The mixture was then poured into a separatory funnel containing 1:1 CH₂Cl₂:5% NaHCO₃ and extracted. The aqueous layer was extracted one more time with CH₂Cl₂ and the combined organics were washed with H₂O, dried with MgSO₄ and concentrated. The residue was dissolved in 5.0 mL THF and piperidine (0.07 mL, 0.75 mmol, 1.1 equiv) was added and allowed to stir for 1 h. The solution was neutralized with 1 N HCl and extracted twice with diethyl ether. The combined organics were washed with brine, dried with MgSO₄, and concentrated. The residue was dissolved in 5 mL THF and 0.12 g Amberlyst-15 was added. This mixture was heated at 70° C. for 2 h. The heterogeneous solution was filtered and concentrated. The crude material was purified by silica gel chromatography to isolate Isoxazolidine 117 (0.054, 0.15 mmol, 22% yield).

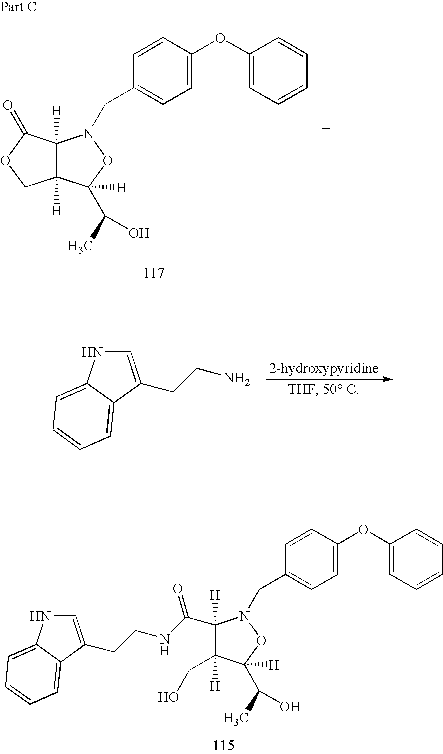

Isoxazolidine 117 (0.054 g, 0.15 mmol, 1.0 equiv) was dissolved in 13.5 mL THF (0.09 M) under a nitrogen atmosphere with stirring. To this solution was added 2-hydroxypyridine (0.074 g, 0.77 mmol, 4.5 equiv) and tryptamine (0.11 g, 0.74 mmol, 4.5 equiv). The solution was stirred at 50° C. for 2 h, quenched with 0.1 N HCl and extracted with ethyl acetate. The aqueous layer was extracted an additional time with ethyl acetate and the combined organics were washed with brine, dried with MgSO₄, and concentrated. The crude material was purified by silica gel chromatography to yield the desired product as a brown solid (0.040 g) which was purified further by HPLC column chromatography. MS (ESI(+)) m/e 515.9 (M+H)⁺.

### Example 81

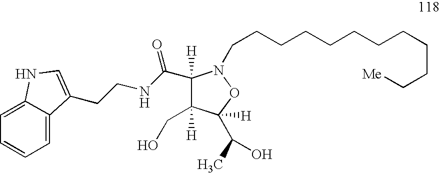

Compound 118 was synthesized according to the procedure described in the synthesis of compound 115 using dodecyl aldehyde in place of 4-phenoxybenzaldehyde. Overall yield 10-25%.

### Example 82

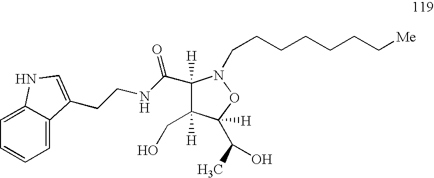

Compound 119 was synthesized according to the procedure described in the synthesis of compound 115 using 1-octanal in place of 4-phenoxybenzaldehyde. Overall yield 10-25%. MS (ESI(+)) m/e 446.0 (M+H)⁺.

### Example 83

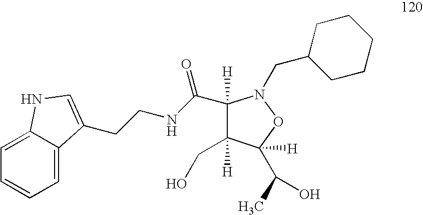

Compound 120 was synthesized according to the procedure described in the synthesis of compound 115 using cyclohexnecarboxaldehyde in place of 4-phenoxybenzaldehyde. Overall yield 10-25%. MS (ESI(+)) m/e 430.8 (M+H)⁺.

### Example 84

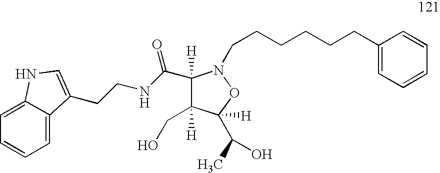

Compound 121 was synthesized according to the procedure described in the synthesis of compound 115 using 6-phenyl-hexanal in place of 4-phenoxybenzaldehyde. Overall yield 10-25%.

### Example 85

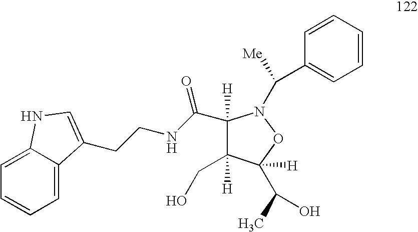

Compound 122 was synthesized according to the procedure described in the synthesis of compound 115 using (R)-N-(1-phenyl-ethyl)-hydroxylamine (Synthesis of this hydroxylamine was described in Tokuyama, H. Organic Synthesis 2003, 80, 207-218) in place of N-(4-phenoxy-benzyl)-hydroxylamine. Overall yield 10-25%. MS (ESI(+)) m/e 438.20 (M+H)⁺.

### Example 86

General procedures for determination of minimum inhibitory concentration (MIC) of isoxazolidine compounds for S. aureus, MRSA, S. pneuomiae, PRSP, E. faecalis, VIRENVRE, and S. cerevisiae Δpdr5 are described below.

Susceptibility testing for bacterial cultures is performed according to the National Council for Clinical Laboratory Standards (NCCLS) protocol M100-S12, “Performance Standards for Antimicrobial Susceptibility Testing.” The bacterial strains recommended by NCCLS were obtained from the American Type Culutre Collection (ATCC) (Table) and were maintained on Mueller Hinton (MHA) agar plates plus or minus 3% lysed horse blood (see Table). The bacteria were grown in Cation adjusted Mueller Hinton Broth (CaMHB) for liquid growth and susceptibility testing. S. cerevisiae (yeast) is grown in yeast peptone dextrose (YPD) media.

All bacterial cultures were grown at 37 degrees Celsius. Fresh colonies of S. aureus and MRSA were inoculated into liquid media and grown overnight and diluted the next day to grow to mid-log (Table 2). E. faecalis and S. pneumonia mid-log cultures were started directly from colony on plates. To attain mid-log growth, cultures were grown to an optical density (OD) at A590 of 0.2-0.8 (see Table 2) and then diluted to proper OD to obtain 5×105 cells/mL.

Diluted bacterial cultures are added to compound plates with compound diluted 2-fold from 100 μg/mL to 0.2 μg/mL final concentration in DMSO. Control antibiotics are also added: vancomycin, linezolid, and penicillin. In 384 well plates, 0.5 μL compound is added followed by addition of 50 μL of culture. DMSO alone is included as a control. Plates incubated at 37 degrees Celsius for 17-24 h.

Yeast cultures are started from a fresh colony into YPD media and then diluted to OD of 0.002. Diluted yeast is added to compound plates as bacteria but incubated at 30 degrees Celsius for 17 h. Controls are amphotericin B and voriconazole.

Plates with cells mixed with compounds are incubated for 17 h for S. aureus and yeast, 24 h for MRSA, and all Enterococcus and Streptococcus strains. Yeast plates are incubated 19 h.

Viability is determined by reading plates at A590 to determine optical density of treated and untreated cells and determining growth inhibition relative to controls.

TABLE I

| | | Strain | | |
|---|---|---|---|---|
| | | or | | |
| Name | Description | ATCC# | Plate | Broth |
| Staph | Staphylococcus aureus | 19636 | MHA | CaMHB |
| | (Smith) | | | |
| MRSA | Methicillin resistant S. aureus | 33591 | MHA | CaMHB |
| Ef | Enterococcus faecalis | 29212 | MHA | CaMHB |
| VRE | Van-resistant E. faecalis | 700221 | MHA | CaMHB |
| VIRE | Van-intermediate E. faecalis | 51299 | MHA | CaMHB |
| Sp | Streptococcus pneumonia | 49619 | MHA-Blood | MHB- |
| | | | | Blood |
| PRSP | Penicillin-res. S. pneumonia | 700671 | MHA-Blood | MHB- |
| | | | | Blood |
| Yeast | S. cerevisiae | Δpdr5* | YPD | YPD |

*obtained from Research Genetics

TABLE 2

Grow to Dilute to

| Assay | OD | OD |
|---|---|---|
| S. aureus | 0.2-0.8 | 0.0005 |
| MRSA | 0.2-0.6 | 0.001 |
| Ef | 0.3-0.6 | 0.001 |
| VRE | 0.3-0.6 | 0.0004 |
| VIRE | 0.3-0.6 | 0.001 |
| Sp | 0.3-0.6 | 0.008 |
| PRSP | 0.3-0.6 | 0.003 |
| Yeast | 0.2-0.9 | 0.002 |

The results from the biological activity analysis of the isoxazolidines of the invention are presented below. Note that “***” indicates that the MIC is less than 5 μg/mL, “**” indicates that the MIC is 5-90 μg/mL, “*” indicates that the MIC is >90 μg/mL, “nd” indicates that the value was not determined.

| | MIC (μg/mL) | | | | | | |
|---|---|---|---|---|---|---|---|
| | | | S. | | | | S. |
| | S. | | pneuo- | | E. | VIRE/ | cerevisiae |
| Structure | aureus | MRSA | miae | PRSP | faecalis | VRE | Δpdr5 |
| 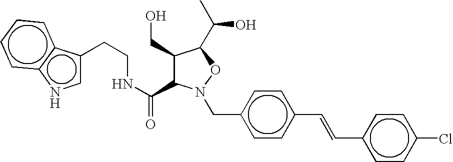 | *** | *** | ** | ** | *** | * | * |
| 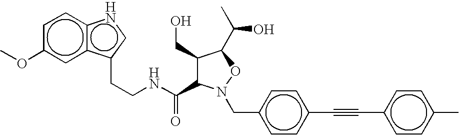 | ** | * | nd | ** | nd | nd | nd |
| 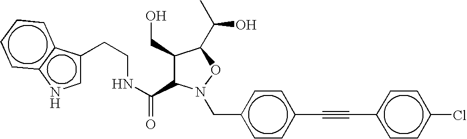 | *** | *** | ** | ** | ** | ** | ** |
| 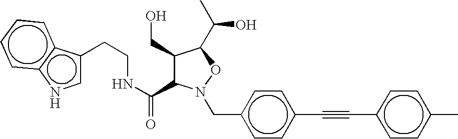 | *** | *** | ** | ** | ** | ** | ** |
| 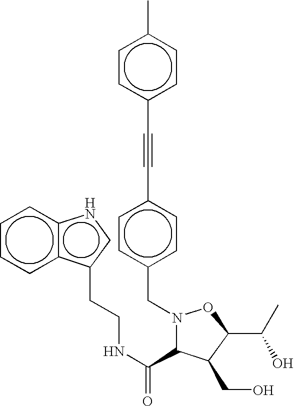 | *** | *** | nd | ** | nd | nd | nd |
| 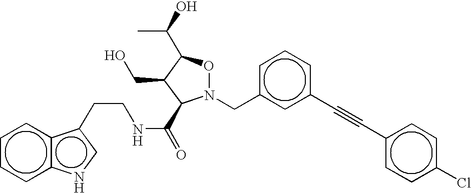 | *** | *** | ** | ** | ** | ** | ** |
| 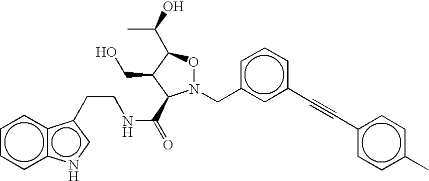 | *** | *** | ** | ** | ** | ** | ** |
| 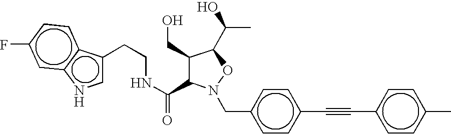 | *** | *** | nd | ** | nd | nd | nd |
| 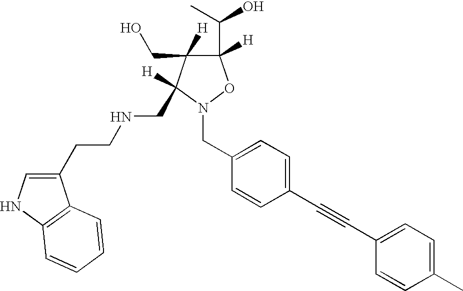 | ** | *** | nd | nd | nd | nd | nd |
| 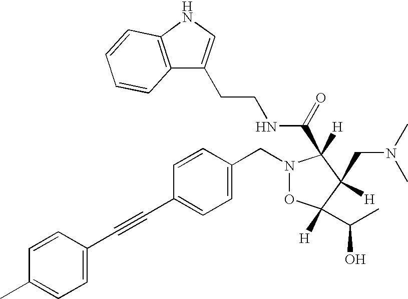 | *** | *** | nd | ** | nd | nd | nd |
| 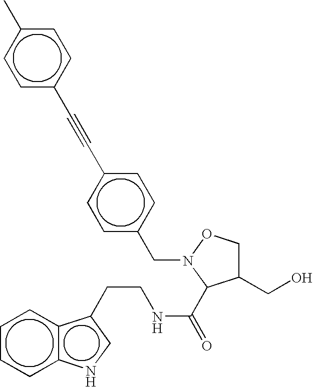 | nd | *** | nd | ** | nd | nd | nd |
| 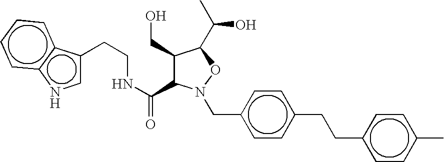 | *** | *** | ** | ** | nd | nd | nd |
| 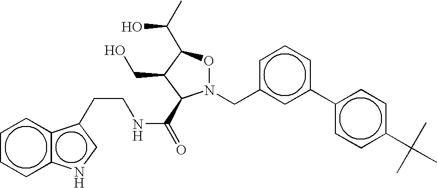 | *** | *** | nd | ** | nd | nd | nd |
| 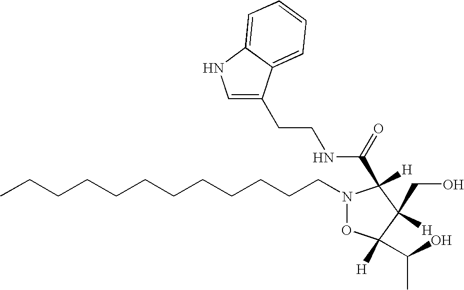 | *** | *** | nd | nd | nd | nd | nd |
| 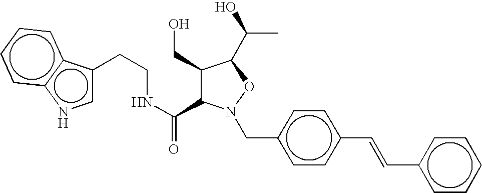 | *** | *** | nd | ** | nd | nd | nd |
| 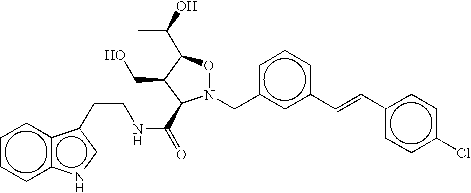 | *** | *** | nd | ** | nd | nd | nd |
| 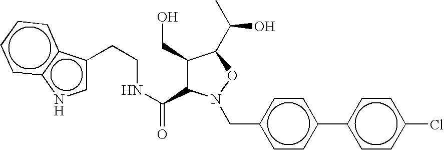 | ** | ** | nd | ** | nd | nd | nd |
| 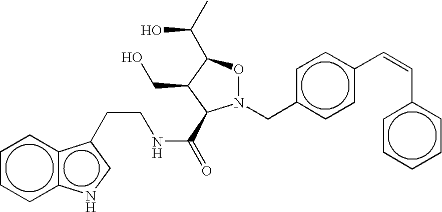 | *** | ** | ** | ** | * | * | nd |
| 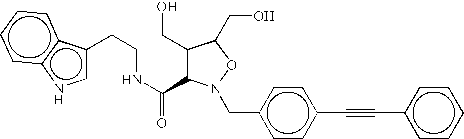 | ** | ** | nd | ** | nd | nd | nd |
| 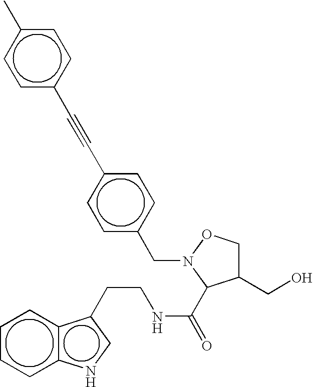 | ** | ** | nd | ** | nd | nd | nd |
| 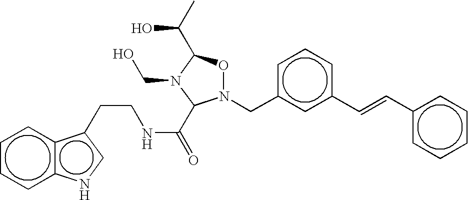 | ** | ** | nd | ** | nd | nd | nd |
| 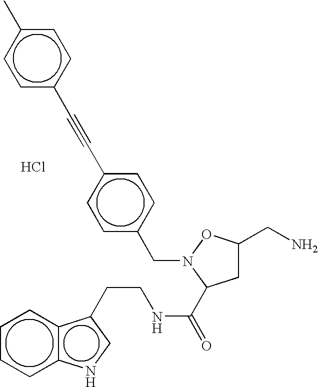 | ** | ** | nd | ** | nd | nd | nd |
| 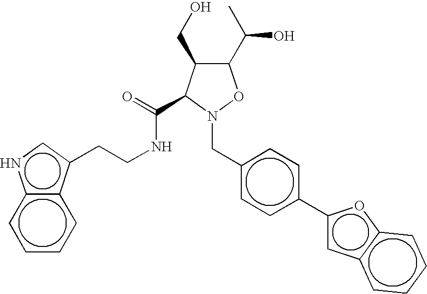 | ** | ** | nd | ** | nd | nd | nd |
| 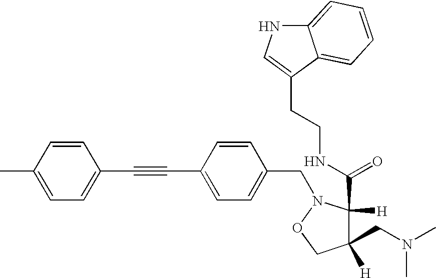 | ** | ** | nd | ** | nd | nd | nd |
| 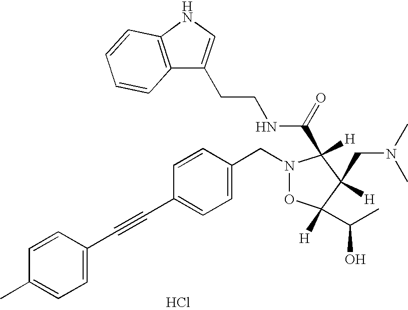 | ** | ** | nd | ** | nd | nd | nd |
| 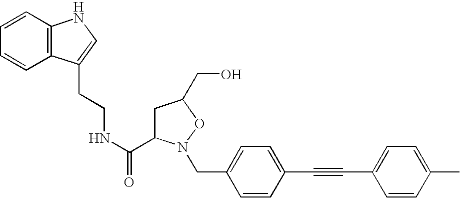 | ** | ** | nd | ** | nd | nd | nd |
| 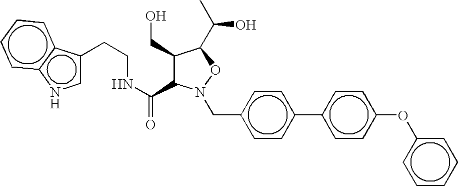 | ** | ** | nd | ** | nd | nd | nd |
| 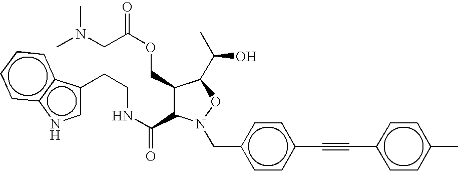 | nd | ** | nd | ** | nd | nd | nd |
| 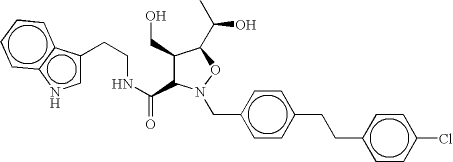 | ** | ** | ** | ** | nd | nd | nd |
| 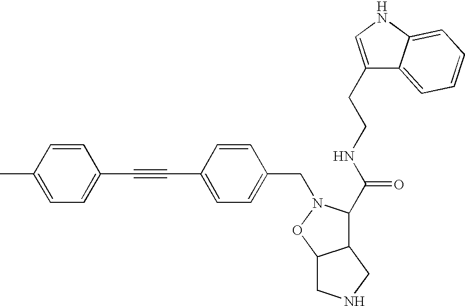 | ** | ** | ** | ** | nd | nd | nd |
| 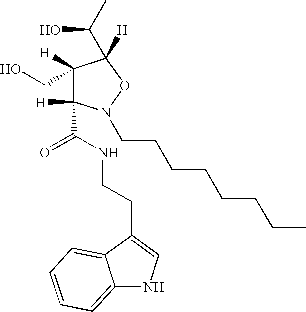 | ** | ** | nd | nd | nd | nd | nd |
| 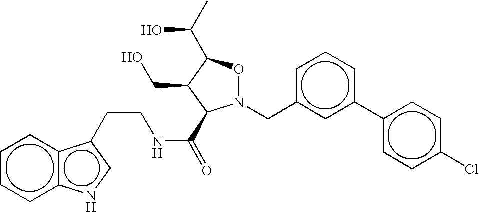 | ** | ** | nd | ** | nd | nd | nd |
| 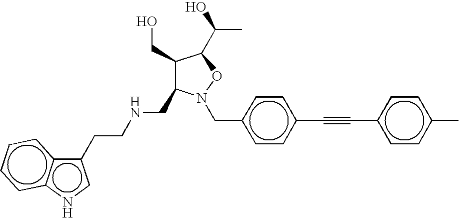 | ** | ** | nd | * | nd | nd | nd |
| 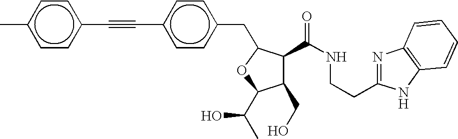 | ** | ** | nd | ** | nd | nd | nd |
| 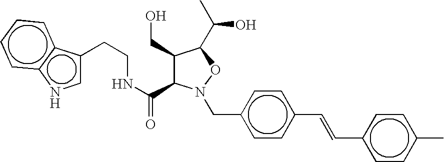 | ** | ** | ** | ** | nd | nd | nd |
| 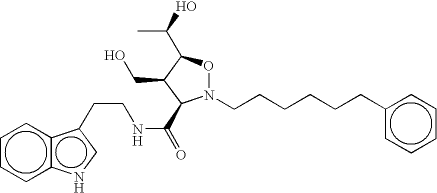 | ** | ** | nd | ** | nd | nd | nd |
| 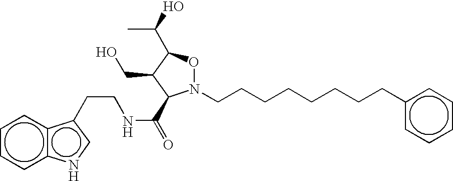 | nd | ** | nd | ** | nd | nd | nd |
| 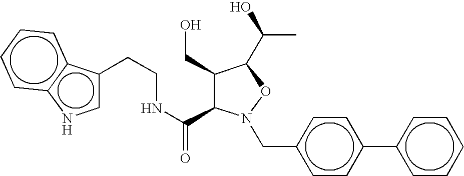 | ** | ** | nd | * | nd | nd | nd |
| 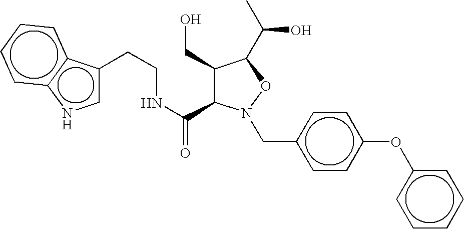 | ** | ** | nd | ** | nd | nd | nd |
| 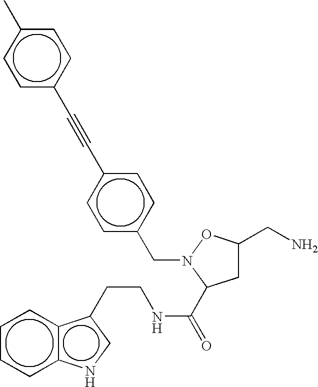 | ** | ** | nd | ** | nd | nd | nd |
| 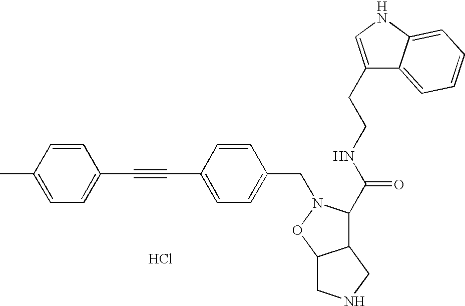 | ** | ** | nd | nd | nd | nd | nd |
| 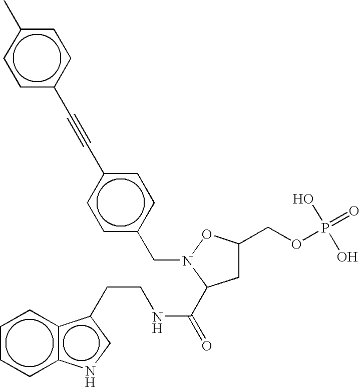 | ** | ** | nd | * | nd | nd | nd |
| 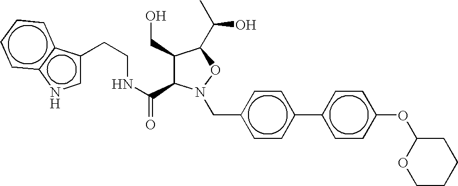 | ** | ** | ** | nd | nd | nd | nd |
| 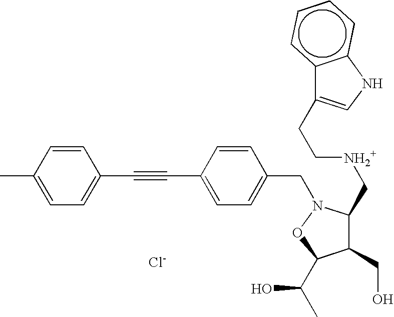 | ** | ** | * | * | nd | nd | nd |
| 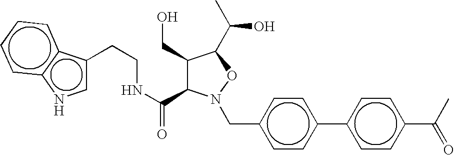 | ** | ** | nd | * | ** | nd | nd |
| 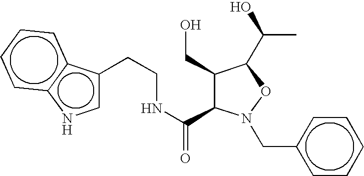 | ** | ** | * | * | ** | ** | ** |
| 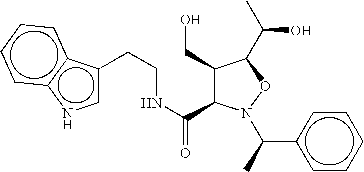 | ** | ** | nd | * | nd | nd | nd |
| 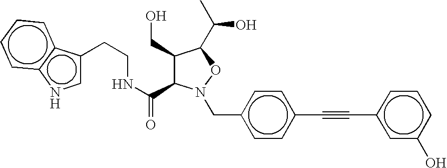 | ** | ** | nd | ** | nd | nd | nd |
| 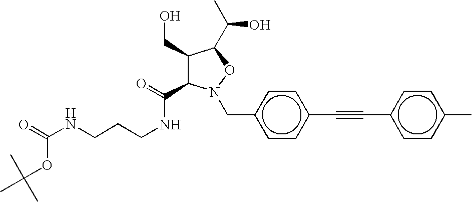 | ** | ** | nd | ** | ** | nd | nd |
| 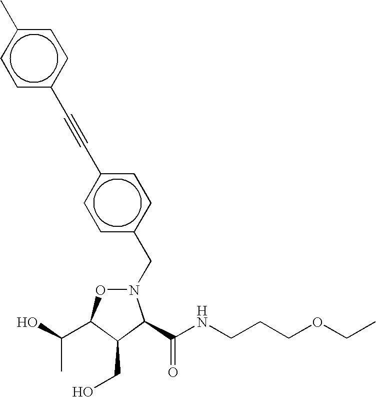 | ** | nd | ** | ** | nd | nd | nd |
| 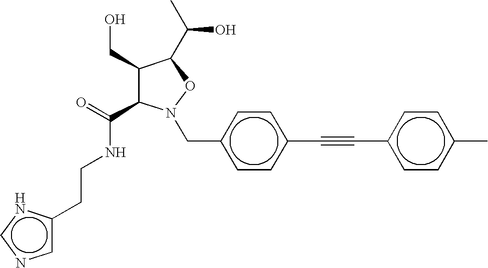 | * | ** | * | nd | nd | nd | nd |
| 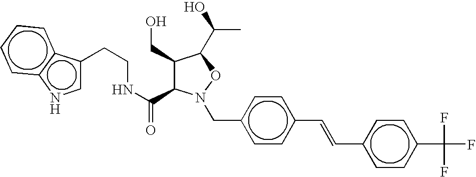 | ** | ** | ** | ** | nd | nd | nd |
| 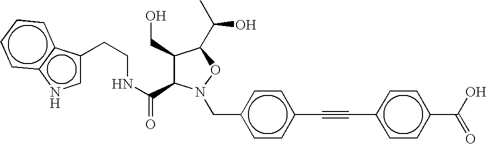 | * | * | nd | * | nd | nd | nd |
| 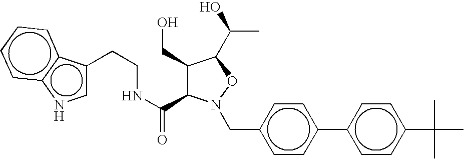 | ** | * | nd | ** | nd | nd | nd |
| 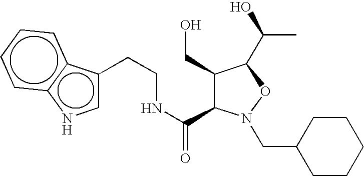 | ** | * | nd | * | ** | nd | nd |
| 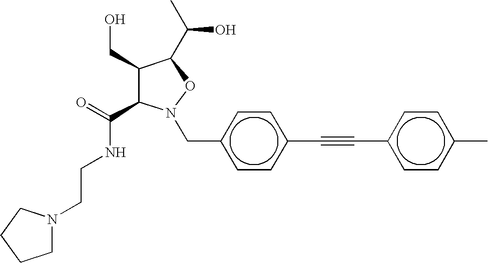 | * | * | nd | * | nd | nd | nd |
| 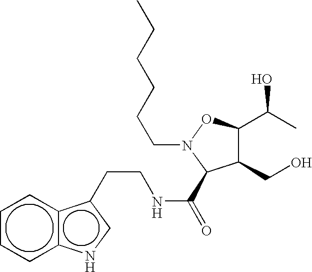 | * | * | nd | * | nd | nd | nd |

### Example 87

The following is a representative example of pharmaceutical dosage forms containing a isoxazolidine compound of the present invention, or a pharmaceutically-acceptable salt thereof, for therapeutic or prophylactic use in humans:

| | |
|---|---|
| (a) Injection I | |
| Compound X | 50% w/v |
| Isotonic aqueous solution | to 100% |
| (b) Injection II (e.g., bolus) | |
| Compound X | 10% w/v |
| Isotonic aqueous solution | to 100% |
| (c) Injection III | |
| Compound X | 5% w/v |
| Isotonic aqueous solution | to 100% |
| (d) Injection IV (e.g., infusion) | |
| Compound X | 1% w/v |
| Isotonic aqueous solution | to 100% |

Buffers, pharmaceutically-acceptable surfactants, oils or cosolvents such as polyethylene glycol, polypropylene glycol, glycerol or ethanol, glidants (such as silicon dioxide) or complexing agents such as a cyclodextrin (for example, hydroxy-propyl .beta.-cyclodextrin or sulfo-butyl-ether beta.-cyclodextrin) may be used to aid formulation. Also, improvements in aqueous solubility, if desired, may be achieved, for example, by conjugation of a compound of the present invention with a phospholipid (such as a (phospho)choline derivative) to form a micellar emulsion.

Note: The above formulations may be obtained by conventional procedures well known in the pharmaceutical art, for example as described in “Remington:The Science & Practice of Pharmacy” Vols. I & II (Ed. A. R. Gennaro (Chairman) et al; Publisher:Mack Publishing Company, Easton, Pa.; 19.sup.th Edition—1995) and “Pharmaceutics—The Science of Dosage Form Design” (Ed. M. E. Aulton; Publisher:Churchill Livingstone; first published 1988). The tablets (a)-(d) may be (polymer) coated by conventional means, for example to provide an enteric coating of cellulose acetate phthalate.

### Example 88

A representative procedure for the synthesis of dipolarophiles for use in [3+2] cycloadditions is described below for the preparation of 2-[4-(4-chloro-phenylethynyl)-benzyl]-5-(1-hydroxy-ethyl)-4-hydroxymethyl-isoxazolidine-3-carboxylic acid [2-(13-indol-3-yl)-ethyl]-amide.

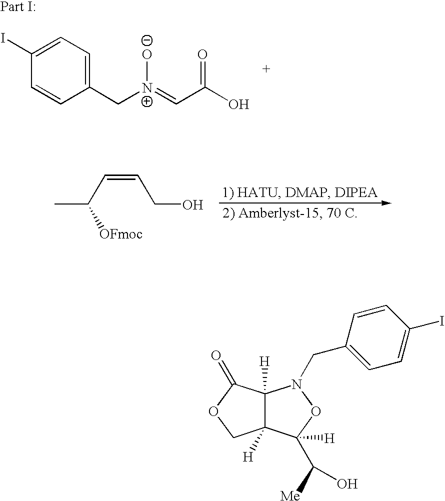

Nitronecarboxylic acid (1.4 g, 3.1 mmol), allylic alcohol (1.0 g, 3.1 mmol), HATU (2.0 g, 6 mmol) and DMAP (0.56 g, 4.6 mmol) were combined with 16 mL of DCM at rt. This cloudy solution was then cooled in an ice-bath and stirred for 1.0 h. Diisopropylethyl amine (0.6 mL, 4.6 mmol) was added drop wise over 15 min. The reaction temperature was then maintained at 0° C. for 1 h. It was then partitioned between 1:1 DCM-5% NaHCO₃ (300 mL). The organic layer was collected and the aqueous layer was back-extracted with an additional 2×125 mL of dichloromethane. The organic layers were combined and washed with 200 mL of water. The organic layer was then dried over MgSO₄, dried and concentrated in vacuo. This residue was dissolved in 10 mL of THF and 0.167 mL of diisopropylethyl amine was added in one portion. It was then heated at reflux for 1 h, and then concentrated in vacuo. The residue was taken up in 8 mL of THF and 1.33 g of Amberlyst-15 was added in one portion. This mixture was heated at 70° C. for 2 h. At which point the reaction mixture was filtered and concentrated. The resulting residue was purified via flash chromatography to yield the desired product in 59% yield.

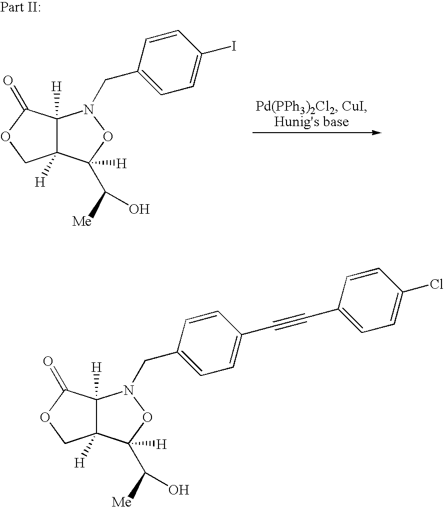

The lactone (100 mg, 0.255 mmol), Pd(PPh₃)₂Cl₂ (54 mg, 0.075 mmol), CuI (19.5 mg, 1.03 mmol), and 1-chloro-4-ethynyl-benzene (70 mg, 0.514 mmol,) were placed in a vial. 4 mL of dimethylformamide and diisopropylethylamine (100 mg, 0.77 mmol) were added to a 15 mL round bottom flask. The flask was flushed with nitrogen and capped with a rubber septa. The reaction mixture was stirred for 3 h and monitored by TLC. The reaction mixture was diluted with 15 mL of ethyl acetate and was washed with 3×20 mL of water. The organic layer was collected, dried over MgSO₄, and concentrated in vacuo. The resulting residue was used in the following reaction without further purification.

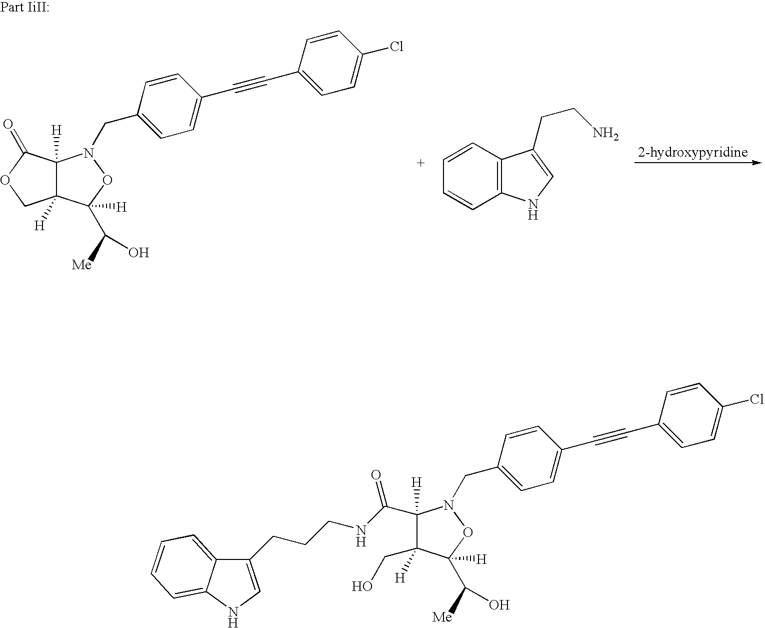

Tryptamine (1.53 g, 3.85 mmol), the lactone (3.08 g, 19.23 mmol), and 2-hydroxypyridine (20 mg, 0.22 mmol) were added to a 5 mL round bottom flask. 1 mL of a 0.35 M solution of 2-hydroxypyridine in THF was added to the round bottom. The round bottom was then flushed with nitrogen and capped with a rubber septa. The reaction mixture was stirred at 50° C. for 5 h. The reaction mixture was then concentrated in vacuo and purified via flash chromatography to yield the desired product in 48% yield over two steps.

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A compound of formula 1: 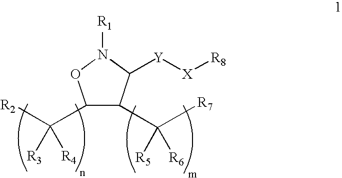
or pharmaceutically acceptable salts, solvates, or hydrates thereof,
wherein
Y is C(R)₂, —C(O)—, —C(S)—, or —S(O)₂—;
R represents independently for each occurrence H, alkyl, cycloalkyl, aralkyl, aryl or heteroaryl;
X is a bond, aryl, or NR₁₀;
m is 0, 1, 2, 3, 4, 5, or 6;
n represents independently for each occurrence 0, 1, 2, 3, 4, 5, or 6;
R₁ is alkyl, aralkyl, heteroaralkyl, has the formula 1a: 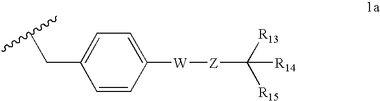
wherein
W is a bond; or bivalent alkyl, alkenyl, or alkynyl chain;
Z is a bond, —(C(R)₂)ₙ—, or —O(C(R)₂)ₙ—, or —CN or —O(C(R)₂)_{q}-alkenyl.
R₁₃ and R₁₄ are independently H, alkyl, alkenyl, alkynyl, aryl, cycloalkyl, heterocycloalkyl, aralkyl, heteroaryl, or heteroaralkyl; or R₁₃ and R₁₄ taken together form a monocyclic or polycyclic ring; or R₁₃ and R₁₄ taken together with R₁₅ form a cycloalkenyl ring;
R₁₅ is halide, hydroxyl, alkoxyl, aryloxy, acyloxy, amino, alkylamino, arylamino, acylamino, aralkylamino, nitro, acylthio, carboxamide, sulfonamide, carboxyl, nitrile, —COR, —CO₂R, —CH₂O-heterocyclyl, or —OR₁₉; or R₁₅ taken together with R₁₃ and R₁₄ form a cycloalkenyl ring;
R₂ and R₇ represent independently H, hydroxyl, alkyl, alkoxyl, —N(R₁₁)₂, acylamino, —CO₂(C(R)₂)ₚC(R)(N(R)₂)CO₂R, —OP(O)(OR₁₂)₂, —N(R)CO₂R, —OC(O)(C(R)₂)_{q}N(R₉)₂, —OC(O)(C(R)₂)_{q}CO₂R, —SO₂N(R)₂, or —OR₁₉; or R₂ and R₇ taken together form an optionally substituted alkyl or heteroalkyl linkage containing 1 to 6 carbon atoms; or R₇ and R₈ taken together form a ring comprising 4 to 7 atoms, of which one, two or three ring atoms may independently be S, O or N;
R₃ and R₆ each represent independently for each occurrence H, hydroxyl, —OC(O)R₉, alkyl, or —OR₁₉;
R₄ and R₅ each represent independently for each occurrence H or alkyl;
R₈ is a branched or unbranched alkyl or alkenyl, cycloalkyl, heterocycloalkyl, bicycloalkyl, a bond to R₇, or has the formula 1c: 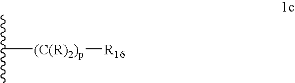
wherein
p is 0, 1, 2, 3, 4, 5, or 6; and
R₁₆ is hydroxyl, aryl, cycloalkyl, cycloalkenyl, heterocycloalkyl, alkoxyl, heteroaryl, —N(R₁₇)₂, —N(R)CO-alkyl, —N(R)CO₂-alkyl, —C(O)N(R₉)aryl, —SO₂N(R)₂, a polycyclic ring containing 8-14 carbon atoms, or —OR₁₉; wherein R₁₇ is independently for each occurrence H, alkyl, aryl, acyl, —(C(R)₂)_{q}OH, or —(C(R)₂)_{q}O(C(R)₂)_{q}OH; or two R₁₇ taken together form a ring;
R₉ and R₁₀ each represent independently for each occurrence H, alkyl, aryl, cycloalkyl, aralkyl, heteroaryl, or heteroaralkyl;
R₁₁ represents independently for each occurrence H, alkyl, cycloalkyl, aryl, or aralkyl;
R₁₂ represents independently for each occurrence H, alkyl, aryl, aralkyl, or an alkali metal; 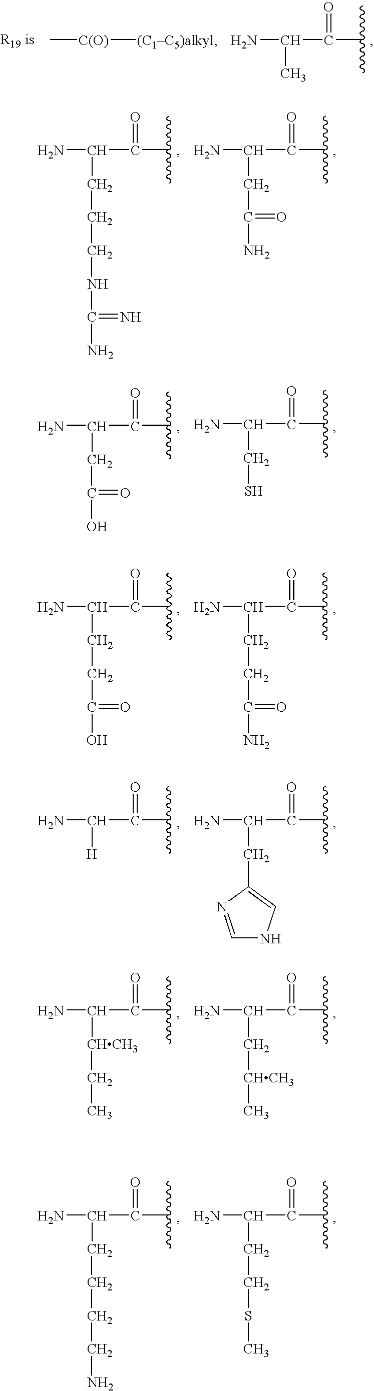 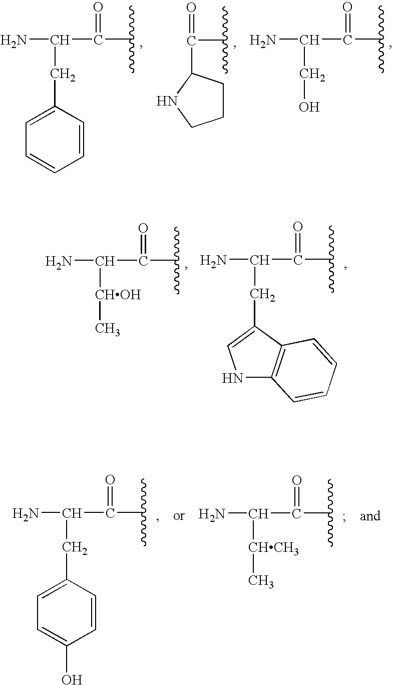
the stereochemical configuration at any stereocenter of a compound represented by 1 is R, S, or a mixture of these configurations.

2. A compound of formula 2: 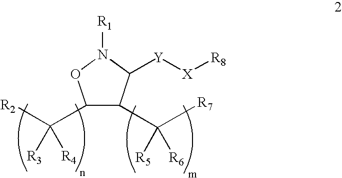
or pharmaceutically acceptable salts, solvates, or hydrates thereof,
wherein
Y is C(R)₂, —C(O)—, —C(S)—, or —S(O)₂—;
R represents independently for each occurrence H, alkyl, cycloalkyl, aralkyl, aryl or heteroaryl;
X is O, S, NR₁₀, or aryl;
m is 0, 1, 2, 3, 4, 5, or 6;
n represents independently for each occurrence 0, 1, 2, 3, 4, 5, or 6;
R₁ is alkyl, aralkyl, heteroaralkyl, has the formula 2a: 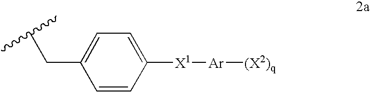
wherein
X¹ is a bond, O, S, amino, alkylamino diradical, alkoxyl diradical, alkyl diradical, alkenyl diradical, alkynyl diradical, amido, sulfonamide, or carbonyl;
Ar is a monocyclic or bicyclic aryl with 6-14 ring atoms; or a monocyclic or bicyclic heteroaryl with 5-14 ring atoms, of which one, two or three ring atoms are independently S, O, or N;
X² represents independently for each occurrence H, hydroxyl, halide, thiol, nitrile, alkyl, fluoroalkyl, —C(O)R₁₈, —CO₂R₁₈, —C(O)N(R₁₈)₂, —SO₂N(R)₂, —O-cycloalkyl, —O-heterocycloalkyl, —O-aryl, or —OR₁₉; and
R₁₈ represents independently for each occurrence H, alkyl, aryl, cycloalkyl, heterocycloalkyl, aralkyl, heteroaryl, or heteroaralkyl; or two instances of R₁₈ taken together form a monocyclic or polycyclic ring;
q represents independently for each occurrence 1, 2, 3, 4, or 5;
R₂ and R₇ represent independently H, hydroxyl, alkyl, halide, alkoxyl, aryloxy, acyloxy, silyloxy, —N(R₁₁)₂, acylamino, —CO₂(C(R)₂)ₚC(R)(N(R)₂)CO₂R, —OP(O)(OR₁₂)₂, —N(R)CO₂R, —OC(O)(C(R)₂)_{q}N(R₉)₂, —OC(O)(C(R)₂)_{q}CO₂R, —SO₂N(R)₂, nitro, sulfhydryl, alkylthio, acylthio, carboxamide, carboxyl, silyl, thioalkyl, alkylsulfonyl, arylsulfonyl, alkylsulfonyloxy, arylsulfonyloxy, ketone, aldehyde, ester, nitrile, —CH₂O-heterocyclyl, or —OR₁₉; or R₂ and R₇ taken together form a —OC(O)O— linkage or an optionally substituted alkyl or heteroalkyl linkage containing 1 to 6 carbon atoms; or R₇ and R₈ taken together form a ring comprising 4 to 7 atoms, of which one, two or three ring atoms may independently be S, O or N;
R₃ and R₆ each represent independently for each occurrence H, halide, hydroxyl, amino, alkyl, aryl, cycloalkyl, aralkyl, heteroaryl, heteroaralkyl, alkoxyl, aryloxy, acyloxy, silyloxy, alkylamino, arylamino, acylamino, sulfonamide, aralklyamino, or —OR₁₉;
R₄ and R₅ each represent independently for each occurrence H, halide, alkyl, aryl, cycloalkyl, aralkyl, heteroaryl, heteroaralkyl, alkoxyl, aryloxy, acyloxy, silyloxy, alkylamino, arylamino, acylamino, sulfonamide, or aralklyamino;
R₈ is a branched or unbranched alkyl or alkenyl, cycloalkyl, heterocycloalkyl, bicycloalkyl, a bond to R₇, or has the formula 2b: 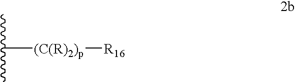
wherein
p is 0, 1, 2, 3, 4, 5, or 6; and
R₁₆ is aryl, cycloalkyl, cycloalkenyl, heterocyclyl, alkoxyl, heteroaryl, N(R₁₇)₂, —N(R)CO₂-alkyl, C(O)N(R₉)aryl, sulfonamide, or a polycyclic ring containing 8-14 carbon atoms;
wherein R₁₇ is independently for each occurrence H, alkyl, aryl or acyl; or two R₁₇ taken together form a ring;
R₉ and R₁₀ each represent independently for each occurrence H, alkyl, aryl, cycloalkyl, aralkyl, heteroaryl, or heteroaralkyl;
R₁₁ represents independently for each occurrence H, alkyl, cycloalkyl, aryl, or aralkyl;
R₁₂ represents independently for each occurrence H, alkyl, aryl, aralkyl, or an alkali metal; 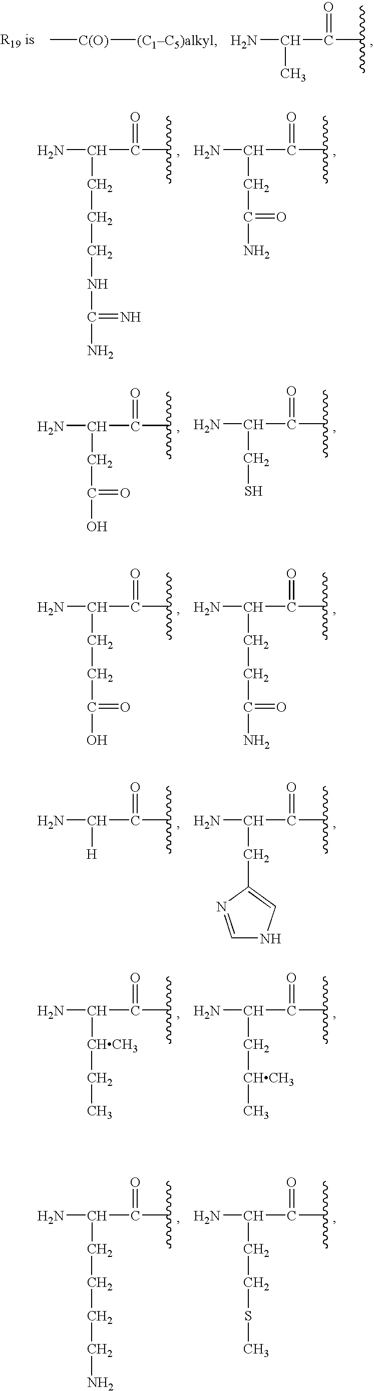 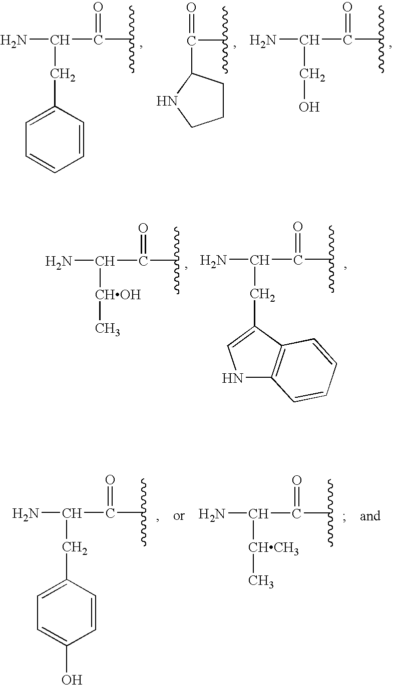
the stereochemical configuration at any stereocenter of a compound represented by 2 is R, S, or a mixture of these configurations.

3. The compound of claim 2, wherein R₂ and R₇ are hydroxyl.

4. The compound of claim 2, wherein R₂ and R₇ are hydroxyl; and R₄, R₅, and R₆ are H.

5. The compound of claim 2, wherein R₂ and R₇ are hydroxyl; R₄, R₅, and R₆ are H; and m and n are 1.

6. The compound of claim 2, wherein R₂ and R₇ are hydroxyl; R₄, R₅, R₆ are H; m and n are 1; and R₃ is methyl.

7. A compound of formula 3: 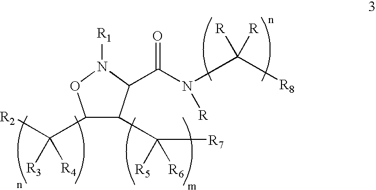
or pharmaceutically acceptable salts, solvates, or hydrates thereof,
wherein
m represents independently for each occurrence 0, 1, 2, 3, 4, 5, or 6;
n represents independently for each occurrence 0, 1, 2, 3, 4, 5, or 6;
R represents independently for each occurrence H, alkyl, cycloalkyl, aralkyl, aryl or heteroaryl;
R₁ is alkyl, aralkyl, heteroaralkyl, has the formula 3a: 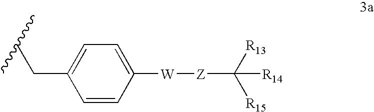
wherein
W is a bond, or bivalent alkyl, alkenyl, or alkynyl chain;
Z is a bond, —(C(R)₂)ₙ—, or —O(C(R)₂)ₙ—;
R₁₃ and R₁₄ are independently H, halide, alkyl, cycloalkyl, alkenyl, alkynyl, aryl, aralkyl, heteroaryl, heteroaralkyl, aminoalkyl, thiol, thioalkyl, silyl, nitro, nitrile, alkoxyl, acyl, acylamino, sulfonamide, —COR, or —CO₂R; or R₁₃ and R₁₄ taken together form a monocyclic or polycyclic ring; or R₁₃ and R₁₄ taken together with R₁₅ form a cycloalkenyl ring; and
R₁₅ is alkyl, cycloalkyl, aryl, heteroaryl, alkenyl, alkynyl, halide, hydroxyl, alkoxyl, aryloxy, acyloxy, silyloxy, amino, alkylamino, arylamino, acylamino, sulfonamide, aralkylamino, nitro, sulfhydryl, alkylthio, acylthio, carboxamide, carboxyl, phosphate, silyl, thioalkyl, alkylsulfonyl, arylsulfonyl, alkylsulfonyloxy, arylsulfonyloxy, nitrile, —COR, —CO₂R, —CH₂O-heterocyclyl, or —OR₁₉; or R₁₅ taken together with R₁₃ and R₁₄ form a cycloalkenyl ring;
R₂ and R₇ each represent independently H, hydroxyl, alkyl, halide, alkoxyl, aryloxy, acyloxy, silyloxy, —N(R₁₁)₂, acylamino, —CO₂(C(R)₂)ₚC(R)(N(R)₂)CO₂R, —OP(O)(OR₁₂)₂, —N(R)CO₂R, —OC(O)(C(R)₂)_{q}N(R₉)₂, —OC(O)(C(R)₂)_{q}CO₂R, —SO₂N(R)₂, nitro, sulfhydryl, alkylthio, acylthio, carboxamide, carboxyl, silyl, thioalkyl, alkylsulfonyl, arylsulfonyl, alkylsulfonyloxy, arylsulfonyloxy, ketone, aldehyde, ester, nitrile, —CH₂O-heterocyclyl, or —OR₁₉; or R₂ and R₇ taken together form a —OC(O)O— linkage or an optionally substituted alkyl or heteroalkyl linkage containing 1 to 6 carbon atoms; or R₇ and R₈ taken together form a ring comprising 4 to 7 atoms, of which one, two or three ring atoms may independently be S, O or N;
R₃ and R₆ each represent independently for each occurrence H, halide, hydroxyl, amino, alkyl, aryl, cycloalkyl, aralkyl, heteroaryl, heteroaralkyl, alkoxyl, aryloxy, acyloxy, silyloxy, alkylamino, arylamino, acylamino, sulfonamide, aralklyamino, or —OR₁₉;
R₄ and R₅ each represent independently for each occurrence H, halide, alkyl, aryl, cycloalkyl, aralkyl, heteroaryl, heteroaralkyl, alkoxyl, aryloxy, acyloxy, silyloxy, alkylamino, arylamino, acylamino, sulfonamide, or aralklyamino;
R₈ is aryl, heteroaryl, heterocycloalkyl, alkoxyl, or —N(R)CO-alkyl;
R₉ and R₁₀ each represent independently for each occurrence H, alkyl, aryl, cycloalkyl, aralkyl, heteroaryl, or heteroaralkyl;
R₁₁ represents independently for each occurrence H, alkyl, cycloalkyl, aryl, or aralkyl;
R₁₂ represents independently for each occurrence H, alkyl, aryl, aralkyl, or an alkali metal; 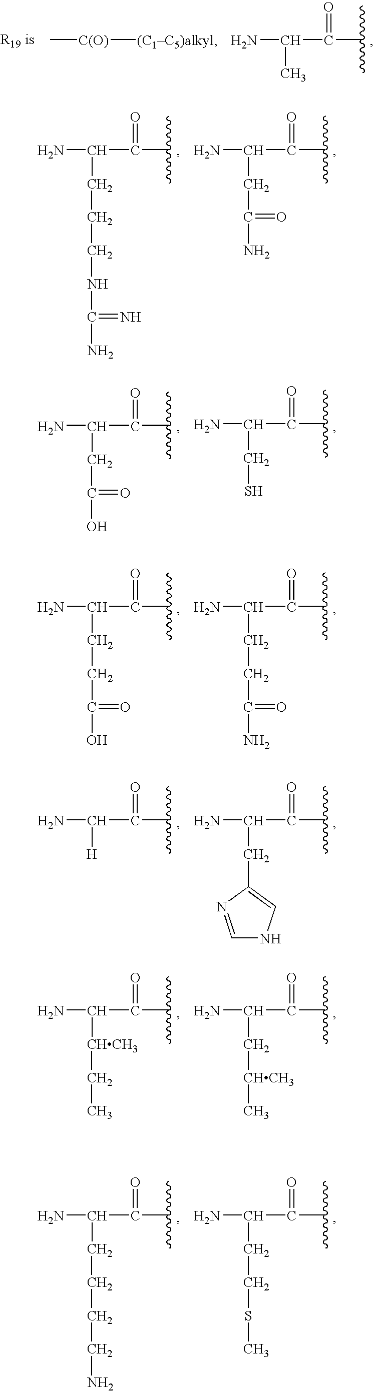 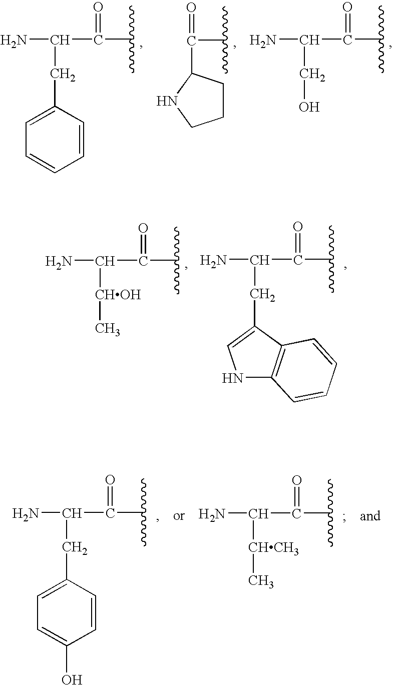
the stereochemical configuration at any stereocenter of a compound represented by 3 is R, S, or a mixture of these configurations.

8. The compound of claim 7, wherein R₈ is selected from the group consisting of 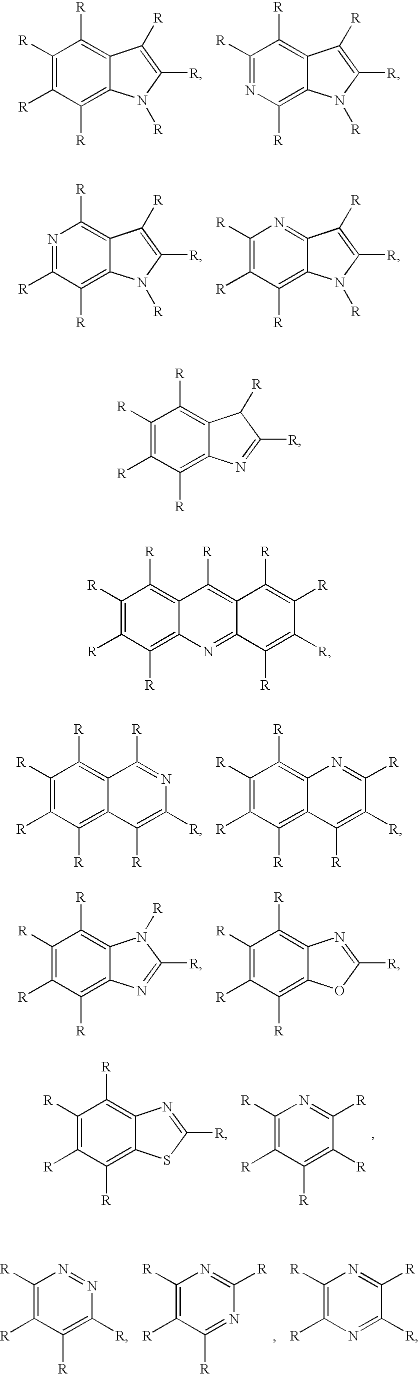 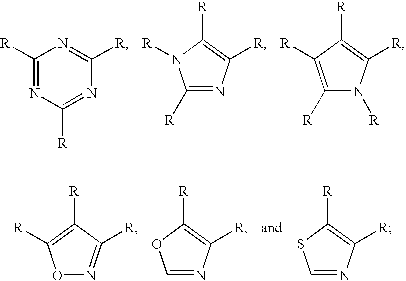
and R represents independently for each occurrence H, alkyl, aryl, or a bond to the CON(R)(CR₂)n-fragment of the compound represented by formula 3.

9. The compound of claim 7, wherein R₈ is 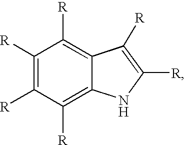
and R represents independently for each occurrence H, alkyl, aryl, or a bond to the CON(R)(CR₂)n-fragment of the compound represented by formula 3.

10. The compoundj of claim 7, wherein R₈ is 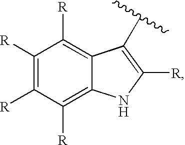
and R represents independently for each occurrence H or alkyl.

11. The compound of claim 7, wherein R₈ is

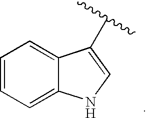

12. A compound of formula 4: 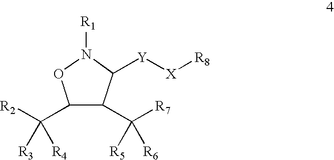
or pharmaceutically acceptable salts, solvates, or hydrates thereof,
wherein
Y is C(R)₂, —C(O)—, —C(S)—, or —S(O)₂—;
X is O, S, or NR₁₀;
R₁ is alkyl, aralkyl, heteroaralkyl, has the formula 4a: 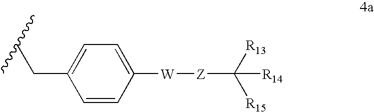
wherein
W is a bond, or bivalent alkyl, alkenyl, or alkynyl chain;
Z is a bond, —(C(R)₂)ₙ—, or —O(C(R)₂)ₙ—, —CN or —O(C(R)₂)_{q}-alkenyl.
R₁₃ and R₁₄ are independently H, alkyl, aryl, cycloalkyl, aralkyl, heteroaryl, or heteroaralkyl; or R₁₃ and R₁₄ taken together form a monocyclic or polycyclic ring; or R₁₃ and R₁₄ taken together with R₁₅ form a cycloalkenyl ring;
R₁₅ is halide, hydroxyl, alkoxyl, aryloxy, acyloxy, silyloxy, amino, alkylamino, arylamino, acylamino, sulfonamide, aralkylamino, nitro, sulfhydryl, alkylthio, acylthio, carboxamide, carboxyl, phosphate, silyl, thioalkyl, alkylsulfonyl, arylsulfonyl, alkylsulfonyloxy, arylsulfonyloxy, nitrile, —COR, —CO₂R, —CH₂O-heterocyclyl, or —OR₁₉; or R₁₅ taken together with R₁₃ and R₁₄ form a cycloalkenyl ring;
R represents independently for each occurrence H, alkyl, cycloalkyl, aralkyl, aryl or heteroaryl;
R₂ and R₇ are independently hydroxyl, —N(R₁₁)₂, —OP(O)(OR₁₂)₂, alkoxyl, —OC(O)(C(R)₂)_{q}N(R₉)₂, or —OR₁₉; or R₂ and R₇ taken together form a —OC(O)O— linkage or an optionally substituted alkyl or heteroalkyl linkage containing 1 to 6 carbon atoms;
R₃ is methyl, ethyl, or propyl;
R₄, R₅, and R₆ are H;
R₈ is a branched or unbranched alkyl or alkenyl; cycloalkyl, heterocycloalkyl, bicycloalkyl, a bond to R₇, or has the formula 4c: 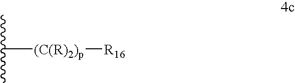
wherein
p is 0, 1, 2, 3, 4, 5, or 6; and
R₁₆ is aryl, cycloalkyl, cycloalkenyl, heterocyclyl, alkoxyl, heteroaryl, —N(R₁₇)₂, —N(R)CO₂-alkyl, sulfonamide, —C(O)N(R₉)aryl, or a polycyclic ring containing 8-14 carbon atoms; wherein R₁₇ is independently for each occurrence H, alkyl, aryl or acyl; or two R₁₇ taken together form a ring;
R₉ and R₁₀ each represent independently for each occurrence H, alkyl, aryl, cycloalkyl, aralkyl, heteroaryl, or heteroaralkyl;
R₁₁ represents independently for each occurrence H, alkyl, cycloalkyl, aryl, or aralkyl;
R₁₂ represents independently for each occurrence H, alkyl, aryl, aralkyl, or an alkali metal; 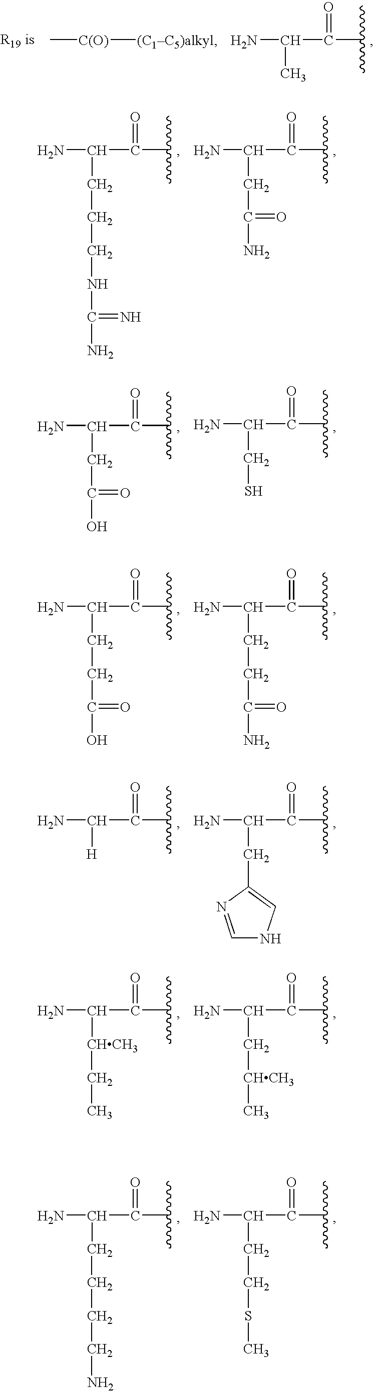 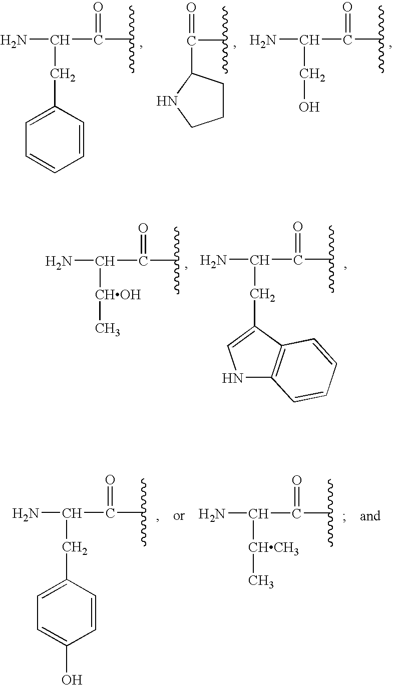
the stereochemical configuration at any stereocenter of a compound represented by 4 is R, S, or a mixture of these configurations.

13. A compound of formula 5: 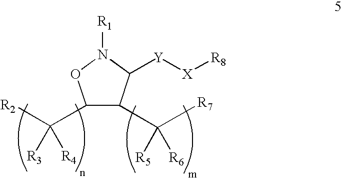
or pharmaceutically acceptable salts, solvates, or hydrates thereof,
wherein
Y is C(R)₂ or —C(O)—;
R represents independently for each occurrence H, alkyl, cycloalkyl, aralkyl, aryl or heteroaryl;
X is NR₁₀;
m is 0, 1, 2, 3, 4, 5, or 6;
n represents independently for each occurrence 0, 1, 2, 3, 4, 5, or 6;
R₁ is alkyl, aralkyl or has the formula 5b: 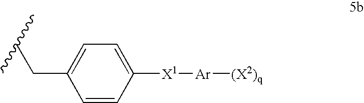
X¹ is a bond, O, alkyl, alkenyl, or alkynyl;
X² is H, halide, hydroxyl, alkyl, fluoroalkyl, —C(O)R₁₈, —CO₂R₁₈, —O-heterocycloalkyl, or —O-aryl; and
R₁₈ represents independently for each occurrence H or alkyl;
Ar represents independently for each occurrence a monocyclic aryl with 6-14 ring atoms;
or a bicyclic heteroaryl with 5-14 ring atoms, of which one, two or three ring atoms are 0;
q represents independently for each occurrence 1, 2, 3, 4, or 5;
R₂ and R₇ represent independently H, hydroxyl, —N(R₁₁)₂, —OP(O)(OR₁₂)₂, or —OC(O)(C(R)₂)_{q}N(R₉)₂; or R₂ and R₇ taken together form an optionally substituted heteroalkyl linkage containing 1 to 6 carbon atoms;
R₃ and R₆ represent independently for each occurrence H;
R₄ and R₅ represent independently for each occurrence H or alkyl;
R₉ and R₁₀ represent independently for each occurrence H or alkyl;
R₈ has the formula 5c: 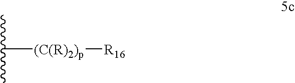
wherein
p is 2 or 3; and
R₁₆ is heterocycloalkyl, alkoxyl, heteroaryl, N(R)CO₂-alkyl, or C(O)N(R₉)aryl; and
the stereochemical configuration at any stereocenter of a compound represented by 5 is R, S, or a mixture of these configurations.

14. The compound of claim 13, wherein R₁ has the formula 5b: 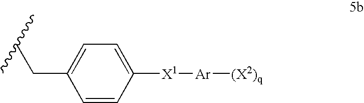
wherein
X¹ is a bond, O, alkyl, alkenyl, or alkynyl;
X² is H, halide, hydroxyl, alkyl, fluoroalkyl, —C(O)R₁₈, —CO₂R₁₈, —O-heterocycloalkyl, or —O-aryl; and
R₁₈ represents independently for each occurrence H or alkyl.

15. The compound of claim 13, wherein R₁ has the formula 5b: 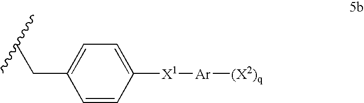
wherein
X¹ is a bond, alkyl, —CH═CH—, or —C≡C—; and
X² is H, halide, hydroxyl, alkyl, —CF₃, —C(O)alkyl, —CO₂H, —O-heterocycloalkyl, or —O-aryl.

16. The compound of claim 13, wherein R₁ has the formula 5b: 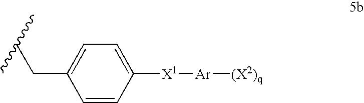
wherein
X¹ is a bond, alkyl, —CH═CH—, or —C≡C—; and
X² is H, chloride, hydroxyl, methyl, t-butyl, —C(O)alkyl, —CO₂H, —O-heterocycloalkyl, or —O-phenyl; and
Ar is phenyl.

17. The compound of claim 13 wherein R₁ is alkyl or aralkyl.

18. The compound of claim 13, wherein R₂ and R₇ are hydroxyl; R₃, R₅, and R₆ are H; R₄ is alkyl; and m and n are 1.

19. The compound of claim 13, wherein R₂ is hydroxyl; R₃, R₅, and Rr are H; R₄ is alkyl; R₇ is alkylamino; and m and n are 1.

20. The compound of claim 13, wherein R₂ is hydroxyl, —N(R₁₁)₂, or —OP(O)(OR₁₂)₂; R₃, R₄, and R₇ are H; n is 1; m is 0; R₁₁ is H; and R₁₂ is H.

21. The compound of claim 13, wherein R₂, R₅, and R₆, are H; n is 0; m is 1; and R₇ is hydroxyl.

22. The compound of claim 13, wherein R₈ has the formula 5c: 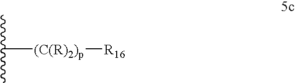
wherein
p is 2 or 3; and
R₁₆ is heteroaryl.

23. The compound of claim 13, wherein R₈ has the formula 5c: 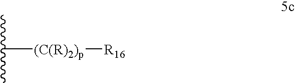
wherein
p is 2 or 3; and
R₁₆ is selected from the group consisting of

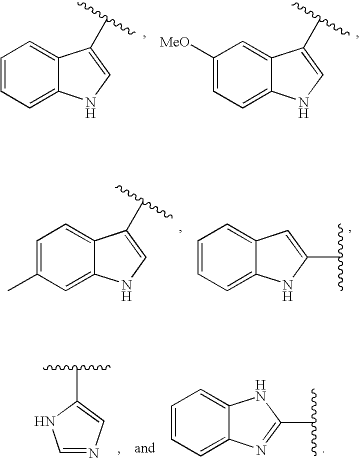

24. The compound of claim 13, wherein R₈ has the formula 5c: 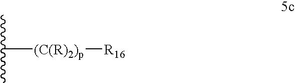
wherein
p is 2 or 3; and
R₁₆ is

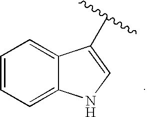

25. The compound of claim 13, wherein R₈ has the formula 5c: 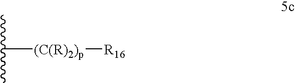
wherein independently for each occurrence:
p is 2 or 3; and
R₁₆ is OEt, —N(H)CO₂-alkyl, or

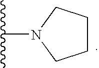

26. The compound of claim 13, wherein R₁ has the formula 5b: 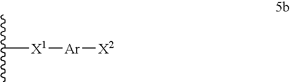
wherein
X¹ is a bond, alkyl, —CH═CH—, or —C≡C—; and
X² is H, chloride, hydroxyl, methyl, t-butyl, —C(O)alkyl, —CO₂H, —O-heterocycloalkyl, or —O-phenyl; and
Ar is phenyl; and
R₈ has the formula 5c: 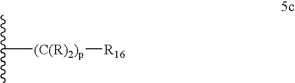
wherein
p is 2 or 3; and
R₁₆ is selected from the group consisting of OEt, —N(H)CO₂-alkyl,

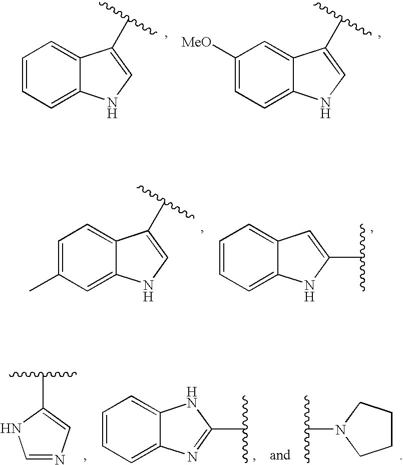

27. The compound of claim 13, wherein R₁ has the formula 5b: 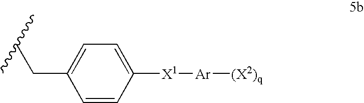
wherein
X¹ is a bond, alkyl, —CH═CH—, or —C≡C—; and
X² is H, chloride, hydroxyl, methyl, t-butyl, —C(O)alkyl, —CO₂H, —O-heterocycloalkyl, or —O-phenyl; and
Ar is phenyl; and
R₈ has the formula 5c: 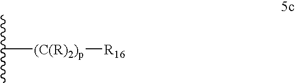
wherein
p is 2 or 3; and
R₁₆ is selected from the group consisting of

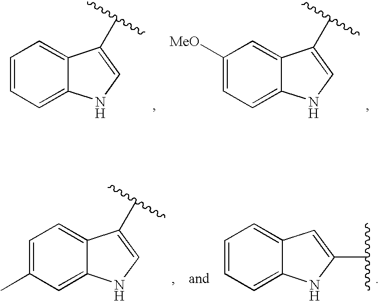

28. A compound selected from the group consisting of

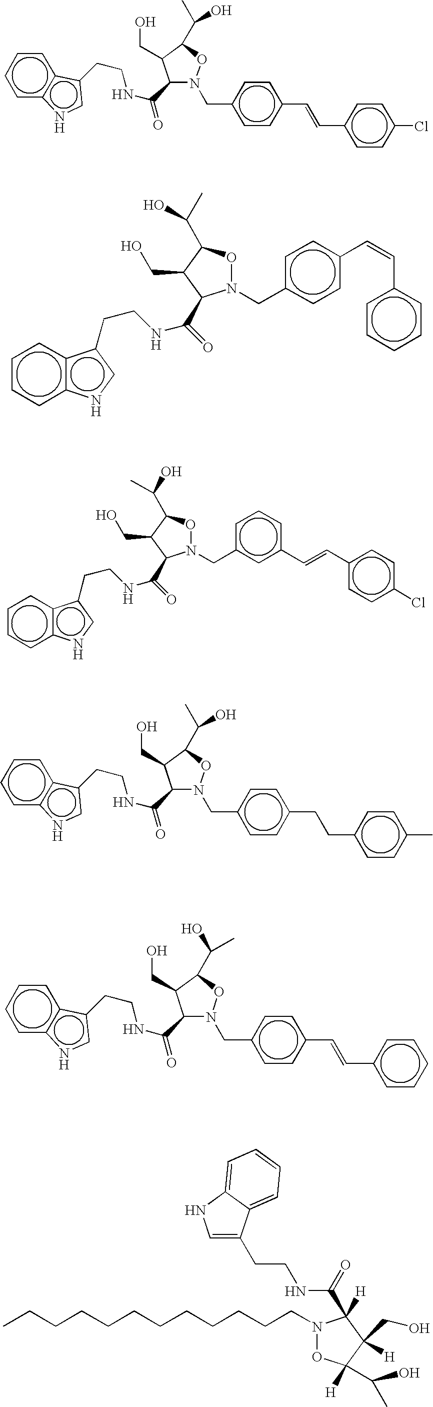

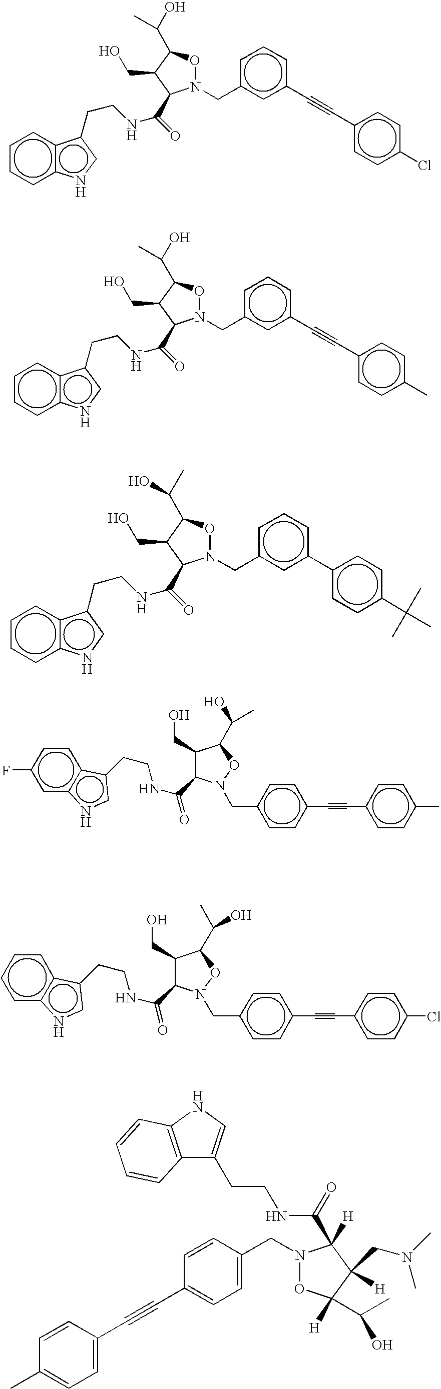

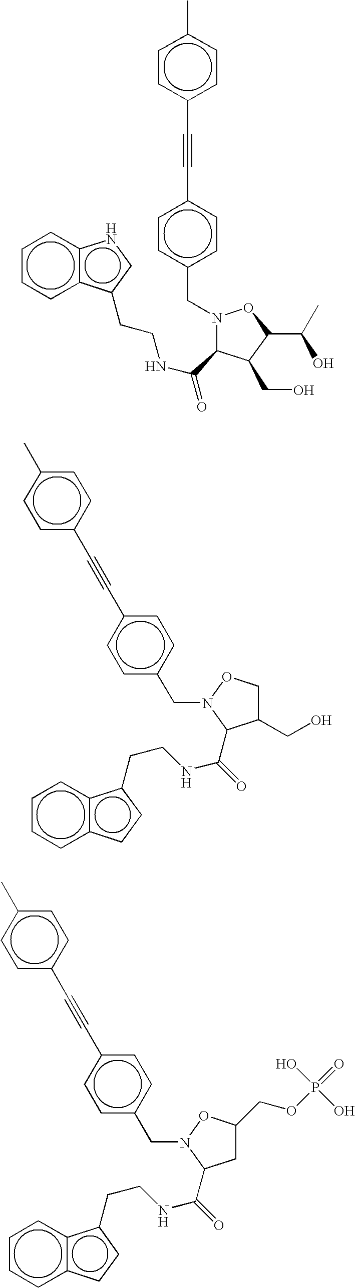

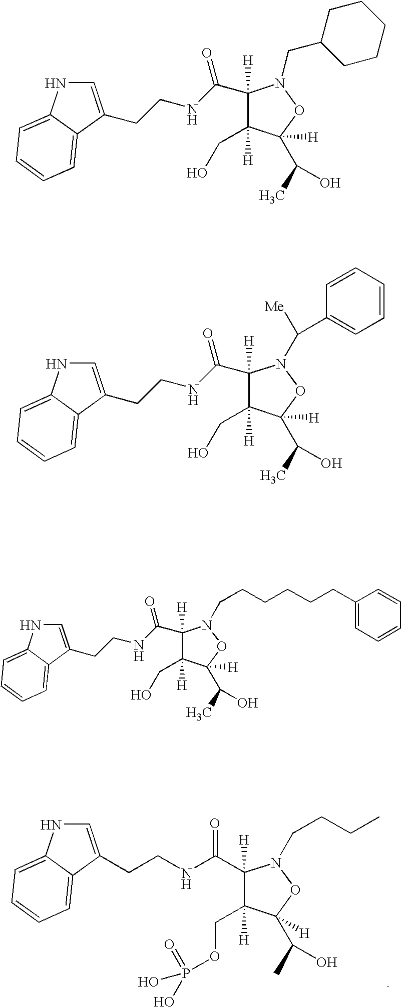

29. A pharmaceutical composition, comprising a compound of any one of claims 1-28; and at least one pharmaceutically acceptable excipient.
